SEQUENCE LISTING (233 sequences)

SEQ ID NO: 1 - protein, 185 aa, synthetic construct
  REGION 1..185: note = source = /note="Description of Artificial Sequence: Synthetic polypeptide"
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 2 - protein, 183 aa, synthetic construct
  REGION 1..183: note = source = /note="Description of Artificial Sequence: Synthetic polypeptide"
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 3 - protein, 182 aa, synthetic construct
  REGION 1..182: note = source = /note="Description of Artificial Sequence: Synthetic polypeptide"
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRSQSRESQC

SEQ ID NO: 4 - protein, 183 aa, synthetic construct
  REGION 1..183: note = source = /note="Description of Artificial Sequence: Synthetic polypeptide"
MDIDPYKEFGATVELLSFLPSDFFPSVRDLLDTASALYREALESPEHCSPHHTALRQAILCWGELMTLATWVGGNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 5 - protein, 184 aa, synthetic construct
  VARIANT 12: note = /replace="S"
  VARIANT 27: note = /replace="I"
  VARIANT 66: note = /replace="N"
  VARIANT 73: note = /replace="S" or "G"
  VARIANT 82: note = /replace="E"
  VARIANT 86: note = /replace="S"
  VARIANT 90: note = /replace="V"
  VARIANT 96: note = /replace="F"
  VARIANT 115: note = /replace="I"
  VARIANT 152..153: note = /replace=" "
  SITE 1..184: note = /note="Variant residues given in the sequence have no preference with respect to those in the annotations for variant positions"
  REGION 1..184: note = source = /note="Description of Artificial Sequence: Synthetic polypeptide"
MDIDPYKEFGATVELLSFLPSDFFPSVDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 6 - protein, 184 aa, synthetic construct
  REGION 1..184: note = source = /note="Description of Artificial Sequence: Synthetic polypeptide"
DIDPYKEFGATVELLSFLPSDFFPSIRDLLDTASALYREALESPEHCSPHHTALRQAILCWGELMTLATWVGSNLEDPASRELVVSYVNVNMGLKIRQLLWFHISCLTFGRETVIEYLVSFGVWIRTPPAYRPPNAPILSTLPETTVVRRRDRGRSPRRRTPSPRRRRSQSPRRRRSQSRESQC

SEQ ID NO: 7 - DNA, 552 bp, synthetic construct
  misc_feature 1..552: note = source = /note="Description of Artificial Sequence: Synthetic polynucleotide"
  source 1..552: mol_type = other DNA
gacatcgacccctacaaggagttcggcgccaccgtggagctgctgagcttcctgcccagcgacttcttccccagcatcagggacctgctggacaccgccagcgccctgtacagggaggccctggagagccccgagcactgcagcccccaccacaccgccctgaggcaggccatcctgtgctggggcgagctgatgaccctggccacctgggtgggcagcaacctggaggaccccgccagcagggagctggtggtgagctacgtgaacgtgaacatgggcctgaagatcaggcagctgctgtggttccacatcagctgcctgaccttcggcagggagaccgtgatcgagtacctggtgagcttcggcgtgtggatcaggaccccccccgcctacaggccccccaacgcccccatcctgagcaccctgcccgagaccaccgtggtgaggaggagggacaggggcaggagccccaggaggaggacccccagccccaggaggaggaggagccagagccccaggaggaggaggagccagagcagggagagccagtgc

SEQ ID NO: 8 - protein, 305 aa, synthetic construct
  REGION 1..305: note = source = /note="Description of Artificial Sequence: Synthetic polypeptide"
PLSYQHFRKLLLLDEEAGPLEEELPRLADEGLNRRVAEDLNLGNLNVSIPWTHKVGNFTGLYSSTVPVFNPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDVSAAFYHIPLHPAAMP

SEQ ID NO: 9 - DNA, 915 bp, synthetic construct
  misc_feature 1..915: note = source = /note="Description of Artificial Sequence: Synthetic polynucleotide"
  source 1..915: mol_type = other DNA
cccctgagctaccagcacttcaggaagctgctgctgctggacgaggaggccggccccctggaggaggagctgcccaggctggccgacgagggcctgaacaggagggtggccgaggacctgaacctgggcaacctgaacgtgagcatcccctggacccacaaggtgggcaacttcaccggcctgtacagcagcaccgtgcccgtgttcaaccccgagtggcagacccccagcttccccaagatccacctgcaggaggacatcgtggacaggtgcaagcagttcgtgggccccctgaccgtgaacgagaagaggaggctgaagctgatcatgcccgccaggttctaccccaacgtgaccaagtacctgcccctggacaagggcatcaagccctactaccccgagcacgccgtgaaccactacttccagaccaggcactacctgcacaccctgtggaaggccggcatcctgtacaagagggagaccaccaggagcgccagcttctgcggcagcccctacagctgggagcaggagctgcagcacggcagctgctggtggctgcagttcaggaacagcaagccctgcagcgagtactgcctgacccacctggtgaacctgctggaggactggggcccctgcgacgagcacggcgagcaccacatcaggatccccaggacccccgccagggtgaccggcggcgtgttcctggtggacaagaacccccacaacaccgccgagagcaggctggtggtggacttcagccagttcagcaggggcatcaccagggtgagctggcccaagttcgccgtgcccaacctgcagagcctgaccaacctgctgagcagcaacctgagctggctgagcctggacgtgagcgccgccttctaccacatccccctgcaccccgccgccatgccc

SEQ ID NO: 10 - protein, 303 aa, synthetic construct
  REGION 1..303: note = source = /note="Description of Artificial Sequence: Synthetic polypeptide"
HLLVGSSGLSRYVARLSSNSRIINHQHGTMQNLHDSCSRNLYVSLLLLYKTFGRKLHLYSHPIILKTKRWGYSLNFMGYVIGSWGSLPQDHIIQKIKECFRKLPVNRPIDWKVCQRIVGLLGFAAPFTQCGYPALMPLYACIQSKQAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADDPSRGRLGLSRPLLRLPFRPTTGRTSLYAVSPSV

SEQ ID NO: 11 - DNA, 909 bp, synthetic construct
  misc_feature 1..909: note = source = /note="Description of Artificial Sequence: Synthetic polynucleotide"
  source 1..909: mol_type = other DNA
cacctgctggtgggcagcagcggcctgagcaggtacgtggccaggctgagcagcaacagcaggatcatcaaccaccagcacggcaccatgcagaacctgcacgacagctgcagcaggaacctgtacgtgagcctgctgctgctgtacaagaccttcggcaggaagctgcacctgtacagccaccccatcatcctgaagaccaagaggtggggctacagcctgaacttcatgggctacgtgatcggcagctggggcagcctgccccaggaccacatcatccagaagatcaaggagtgcttcaggaagctgcccgtgaacaggcccatcgactggaaggtgtgccagaggatcgtgggcctgctgggcttcgccgcccccttcacccagtgcggctaccccgccctgatgcccctgtacgcctgcatccagagcaagcaggccttcaccttcagccccacctacaaggccttcctgagcaagcagtacctgaacctgtaccccgtggccaggcagaggcccggcctgtgccaggtgttcgccgacgccacccccaccggctggggcctggccatgggccaccagaggatgaggggcaccttcgtggcccccctgcccatccacaccgccgagctgctggccgcctgcttcgccaggagcaggagcggcgccaagatcctgggcaccgacaacagcgtggtgctgagcaggaagtacaccagcttcccctggctgctgggctgcgccgccaactggatcctgaggggcaccagcttcgtgtacgtgcccagcgccctgaaccccgccgacgaccccagcaggggcaggctgggcctgagcaggcccctgctgaggctgcccttcaggcccaccaccggcaggaccagcctgtacgccgtgagccccagcgtg

SEQ ID NO: 12 - protein, 269 aa, synthetic construct
  REGION 1..269: note = source = /note="Description of Artificial Sequence: Synthetic polypeptide"
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGP

SEQ ID NO: 13 - protein, 127 aa, synthetic construct
  REGION 1..127: note = source = /note="Description of Artificial Sequence: Synthetic polypeptide"
GPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 14 - protein, 580 aa, synthetic construct
  REGION 1..580: note = source = /note="Description of Artificial Sequence: Synthetic polypeptide"
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPDIDPYKEFGATVELLSFLPSDFFPSIRDLLDTASALYREALESPEHCSPHHTALRQAILCWGELMTLATWVGSNLEDPASRELVVSYVNVNMGLKIRQLLWFHISCLTFGRETVIEYLVSFGVWIRTPPAYRPPNAPILSTLPETTVVRRRDRGRSPRRRTPSPRRRRSQSPRRRRSQSRESQCGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 15 - DNA, 1743 bp, synthetic construct
  misc_feature 1..1743: note = source = /note="Description of Artificial Sequence: Synthetic polynucleotide"
  source 1..1743: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccgacatcgacccctacaaggagttcggcgccaccgtggagctgctgagcttcctgcccagcgacttcttccccagcatcagggacctgctggacaccgccagcgccctgtacagggaggccctggagagccccgagcactgcagcccccaccacaccgccctgaggcaggccatcctgtgctggggcgagctgatgaccctggccacctgggtgggcagcaacctggaggaccccgccagcagggagctggtggtgagctacgtgaacgtgaacatgggcctgaagatcaggcagctgctgtggttccacatcagctgcctgaccttcggcagggagaccgtgatcgagtacctggtgagcttcggcgtgtggatcaggaccccccccgcctacaggccccccaacgcccccatcctgagcaccctgcccgagaccaccgtggtgaggaggagggacaggggcaggagccccaggaggaggacccccagccccaggaggaggaggagccagagccccaggaggaggaggagccagagcagggagagccagtgcgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttactag

SEQ ID NO: 16 - protein, 701 aa, synthetic construct
  REGION 1..701: note = source = /note="Description of Artificial Sequence: Synthetic polypeptide"
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPPLSYQHFRKLLLLDEEAGPLEEELPRLADEGLNRRVAEDLNLGNLNVSIPWTHKVGNFTGLYSSTVPVFNPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDVSAAFYHIPLHPAAMPGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 17 - DNA, 2106 bp, synthetic construct
  misc_feature 1..2106: note = source = /note="Description of Artificial Sequence: Synthetic polynucleotide"
  source 1..2106: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccccctgagctaccagcacttcaggaagctgctgctgctggacgaggaggccggccccctggaggaggagctgcccaggctggccgacgagggcctgaacaggagggtggccgaggacctgaacctgggcaacctgaacgtgagcatcccctggacccacaaggtgggcaacttcaccggcctgtacagcagcaccgtgcccgtgttcaaccccgagtggcagacccccagcttccccaagatccacctgcaggaggacatcgtggacaggtgcaagcagttcgtgggtcccctgaccgtgaacgagaagaggaggctgaagctgatcatgcccgccaggttctaccccaacgtgaccaagtacctgcccctggacaagggcatcaagccctactaccccgagcacgccgtgaaccactacttccagaccaggcactacctgcacaccctgtggaaggccggcatcctgtacaagagggagaccaccaggagcgccagcttctgcggcagcccctacagctgggagcaggagctgcagcacggcagctgctggtggctgcagttcaggaacagcaagccctgcagcgagtactgcctgacccacctggtgaacctgctggaggactggggtccctgcgacgagcacggcgagcaccacatcaggatccccaggacccccgccagggtgaccggcggcgtgttcctggtggacaagaacccccacaacaccgccgagagcaggctggtggtggacttcagccagttcagcaggggcatcaccagggtgagctggcccaagttcgccgtgcccaacctgcagagcctgaccaacctgctgagcagcaacctgagctggctgagcctggacgtgagcgccgccttctaccacatccccctgcaccccgccgccatgcccgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttactag

SEQ ID NO: 18 - protein, 699 aa, synthetic construct
  REGION 1..699: note = source = /note="Description of Artificial Sequence: Synthetic polypeptide"
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHLLVGSSGLSRYVARLSSNSRIINHQHGTMQNLHDSCSRNLYVSLLLLYKTFGRKLHLYSHPIILKTKRWGYSLNFMGYVIGSWGSLPQDHIIQKIKECFRKLPVNRPIDWKVCQRIVGLLGFAAPFTQCGYPALMPLYACIQSKQAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADDPSRGRLGLSRPLLRLPFRPTTGRTSLYAVSPSVGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 19 - DNA, 2100 bp, synthetic construct
  misc_feature 1..2100: note = source = /note="Description of Artificial Sequence: Synthetic polynucleotide"
  source 1..2100: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccacctgctggtgggcagcagcggcctgagcaggtacgtggccaggctgagcagcaacagcaggatcatcaaccaccagcacggcaccatgcagaacctgcacgacagctgcagcaggaacctgtacgtgagcctgctgctgctgtacaagaccttcggcaggaagctgcacctgtacagccaccccatcatcctgaagaccaagaggtggggctacagcctgaacttcatgggctacgtgatcggcagctggggcagcctgccccaggaccacatcatccagaagatcaaggagtgcttcaggaagctgcccgtgaacaggcccatcgactggaaggtgtgccagaggatcgtgggcctgctgggcttcgccgcccccttcacccagtgcggctaccccgccctgatgcccctgtacgcctgcatccagagcaagcaggccttcaccttcagccccacctacaaggccttcctgagcaagcagtacctgaacctgtaccccgtggccaggcagaggcccggcctgtgccaggtgttcgccgacgccacccccaccggctggggcctggccatgggccaccagaggatgaggggcaccttcgtggcccccctgcccatccacaccgccgagctgctggccgcctgcttcgccaggagcaggagcggcgccaagatcctgggcaccgacaacagcgtggtgctgagcaggaagtacaccagcttcccctggctgctgggctgcgccgccaactggatcctgaggggcaccagcttcgtgtacgtgcccagcgccctgaaccccgccgacgaccccagcaggggcaggctgggcctgagcaggcccctgctgaggctgcccttcaggcccaccaccggcaggaccagcctgtacgccgtgagccccagcgtggggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttactag

SEQ ID NO: 20 - protein, 15 aa, synthetic construct
  REGION 1..15: note = source = /note="Description of Artificial Sequence: Synthetic peptide"
DIDPYKEFGATVELL

SEQ ID NO: 21 - protein, 15 aa, synthetic construct
  REGION 1..15: note = source = /note="Description of Artificial Sequence: Synthetic peptide"
KEFGATVELLSFLPS

SEQ ID NO: 22 - protein, 15 aa, synthetic construct
  REGION 1..15: note = source = /note="Description of Artificial Sequence: Synthetic peptide"
TVELLSFLPSDFFPS

SEQ ID NO: 23 - protein, 15 aa, synthetic construct
  REGION 1..15: note = source = /note="Description of Artificial Sequence: Synthetic peptide"
SFLPSDFFPSIRDLL

SEQ ID NO: 24 - protein, 15 aa, synthetic construct
  REGION 1..15: note = source = /note="Description of Artificial Sequence: Synthetic peptide"
DFFPSIRDLLDTASA

SEQ ID NO: 25 - protein, 15 aa, synthetic construct
  REGION 1..15: note = source = /note="Description of Artificial Sequence: Synthetic peptide"
IRDLLDTASALYREA

SEQ ID NO: 26 - protein, 15 aa, synthetic construct
  REGION 1..15: note = source = /note="Description of Artificial Sequence: Synthetic peptide"
DTASALYREALESPE

SEQ ID NO: 27 - protein, 15 aa, synthetic construct
  REGION 1..15: note = source = /note="Description of Artificial Sequence: Synthetic peptide"
LYREALESPEHCSPH

SEQ ID NO: 28 - protein, 15 aa, synthetic construct
  REGION 1..15: note = source = /note="Description of Artificial Sequence: Synthetic peptide"
LESPEHCSPHHTALR

SEQ ID NO: 29 - protein, 15 aa, synthetic construct
  REGION 1..15: note = source = /note="Description of Artificial Sequence: Synthetic peptide"
HCSPHHTALRQAILC

SEQ ID NO: 30 - protein, 15 aa, synthetic construct
  REGION 1..15: note = source = /note="Description of Artificial Sequence: Synthetic peptide"
HTALRQAILCWGELM

SEQ ID NO: 31 - protein, 15 aa, synthetic construct
  REGION 1..15: note = source = /note="Description of Artificial Sequence: Synthetic peptide"
QAILCWGELMTLATW

SEQ ID NO: 32 - protein, 15 aa, synthetic construct
  REGION 1..15: note = source = /note="Description of Artificial Sequence: Synthetic peptide"
WGELMTLATWVGSNL

SEQ ID NO: 33 - protein, 15 aa, synthetic construct
  REGION 1..15: note = source = /note="Description of Artificial Sequence: Synthetic peptide"
TLATWVGSNLEDPAS

SEQ ID NO: 34 - protein, 15 aa, synthetic construct
  REGION 1..15: note = source = /note="Description of Artificial Sequence: Synthetic peptide"
VGSNLEDPASRELVV

SEQ ID NO: 35 - protein, 15 aa, synthetic construct
  REGION 1..15: note = source = /note="Description of Artificial Sequence: Synthetic peptide"
EDPASRELVVSYVNV

SEQ ID NO: 36 - protein, 15 aa, synthetic construct
  REGION 1..15: note = source = /note="Description of Artificial Sequence: Synthetic peptide"
RELVVSYVNVNMGLK

SEQ ID NO: 37 - protein, 15 aa, synthetic construct
  REGION 1..15: note = source = /note="Description of Artificial Sequence: Synthetic peptide"
SYVNVNMGLKIRQLL

SEQ ID NO: 38 - protein, 15 aa, synthetic construct
  REGION 1..15: note = source = /note="Description of Artificial Sequence: Synthetic peptide"
NMGLKIRQLLWFHIS

SEQ ID NO: 39 - protein, 15 aa, synthetic construct
  REGION 1..15: note = source = /note="Description of Artificial Sequence: Synthetic peptide"
IRQLLWFHISCLTFG

SEQ ID NO: 40 - protein, 15 aa, synthetic construct
  REGION 1..15: note = source = /note="Description of Artificial Sequence: Synthetic peptide"
WFHISCLTFGRETVI

SEQ ID NO: 41 - protein, 15 aa, synthetic construct
  REGION 1..15: note = source = /note="Description of Artificial Sequence: Synthetic peptide"
CLTFGRETVIEYLVS

SEQ ID NO: 42 - protein, 15 aa, synthetic construct
  REGION 1..15: note = source = /note="Description of Artificial Sequence: Synthetic peptide"
RETVIEYLVSFGVWI

SEQ ID NO: 43 - protein, 15 aa, synthetic construct
  REGION 1..15: note = source = /note="Description of Artificial Sequence: Synthetic peptide"
EYLVSFGVWIRTPPA

SEQ ID NO: 44 - protein, 15 aa, synthetic construct
  REGION 1..15: note = source = /note="Description of Artificial Sequence: Synthetic peptide"
FGVWIRTPPAYRPPN

SEQ ID NO: 45 - protein, 15 aa, synthetic construct
  REGION 1..15: note = source = /note="Description of Artificial Sequence: Synthetic peptide"
RTPPAYRPPNAPILS

SEQ ID NO: 46 - protein, 15 aa, synthetic construct
  REGION 1..15: note = source = /note="Description of Artificial Sequence: Synthetic peptide"
YRPPNAPILSTLPET

SEQ ID NO: 47 - protein, 15 aa, synthetic construct
  REGION 1..15: note = source = /note="Description of Artificial Sequence: Synthetic peptide"
APILSTLPETTVVRR

SEQ ID NO: 48 - protein, 15 aa, synthetic construct
  REGION 1..15: note = source = /note="Description of Artificial Sequence: Synthetic peptide"
TLPETTVVRRRDRGR

SEQ ID NO: 49 - protein, 15 aa, synthetic construct
  REGION 1..15: note = source = /note="Description of Artificial Sequence: Synthetic peptide"
TVVRRRDRGRSPRRR

SEQ ID NO: 50 - protein, 15 aa, synthetic construct
  REGION 1..15: note = source = /note="Description of Artificial Sequence: Synthetic peptide"
RDRGRSPRRRTPSPR

SEQ ID NO: 51 - protein, 15 aa, synthetic construct
  REGION 1..15: note = source = /note="Description of Artificial Sequence: Synthetic peptide"
SPRRRTPSPRRRRSQ

SEQ ID NO: 52 - protein, 15 aa, synthetic construct
  REGION 1..15: note = source = /note="Description of Artificial Sequence: Synthetic peptide"
TPSPRRRRSQSPRRR

SEQ ID NO: 53 - protein, 16 aa, synthetic construct
  REGION 1..16: note = source = /note="Description of Artificial Sequence: Synthetic peptide"
RRRSQSPRRRRRSQSR

SEQ ID NO: 54 - protein, 15 aa, synthetic construct
  REGION 1..15: note = source = /note="Description of Artificial Sequence: Synthetic peptide"
SPRRRRRSQSRESQC

SEQ ID NO: 55 - protein, 15 aa, synthetic construct
  REGION 1..15: note = source = /note="Description of Artificial Sequence: Synthetic peptide"
PLSYQHFRKLLLLDE

SEQ ID NO: 56 - protein, 15 aa, synthetic construct
  REGION 1..15: note = source = /note="Description of Artificial Sequence: Synthetic peptide"
HFRKLLLLDEEAGPL

SEQ ID NO: 57 - protein, 15 aa, synthetic construct
  REGION 1..15: note = source = /note="Description of Artificial Sequence: Synthetic peptide"
LLLDEEAGPLEEELP

SEQ ID NO: 58 - protein, 15 aa, synthetic construct
  REGION 1..15: note = source = /note="Description of Artificial Sequence: Synthetic peptide"
EAGPLEEELPRLADE

SEQ ID NO: 59 - protein, 15 aa, synthetic construct
  REGION 1..15: note = source = /note="Description of Artificial Sequence: Synthetic peptide"
EEELPRLADEGLNRR

SEQ ID NO: 60 - protein, 15 aa, synthetic construct
  REGION 1..15: note = source = /note="Description of Artificial Sequence: Synthetic peptide"
RLADEGLNRRVAEDL

SEQ ID NO: 61 - protein, 15 aa, synthetic construct
  REGION 1..15: note = source = /note="Description of Artificial Sequence: Synthetic peptide"
GLNRRVAEDLNLGNL

SEQ ID NO: 62 - protein, 15 aa, synthetic construct
  REGION 1..15: note = source = /note="Description of Artificial Sequence: Synthetic peptide"
VAEDLNLGNLNVSIP

SEQ ID NO: 63 - protein, 15 aa, synthetic construct
  REGION 1..15: note = source = /note="Description of Artificial Sequence: Synthetic peptide"
NLGNLNVSIPWTHKV

SEQ ID NO: 64 - protein, 15 aa, synthetic construct
  REGION 1..15: note = source = /note="Description of Artificial Sequence: Synthetic peptide"
NVSIPWTHKVGNFTG

SEQ ID NO: 65 - protein, 15 aa, synthetic construct
  REGION 1..15: note = source = /note="Description of Artificial Sequence: Synthetic peptide"
WTHKVGNFTGLYSST

SEQ ID NO: 66 - protein, 15 aa, synthetic construct
  REGION 1..15: note = source = /note="Description of Artificial Sequence: Synthetic peptide"
GNFTGLYSSTVPVFN

SEQ ID NO: 67 - protein, 15 aa, synthetic construct
  REGION 1..15: note = source = /note="Description of Artificial Sequence: Synthetic peptide"
LYSSTVPVFNPEWQT

SEQ ID NO: 68 - protein, 15 aa, synthetic construct
  REGION 1..15: note = source = /note="Description of Artificial Sequence: Synthetic peptide"
VPVFNPEWQTPSFPK

SEQ ID NO: 69 - protein, 16 aa, synthetic construct
  REGION 1..16: note = source = /note="Description of Artificial Sequence: Synthetic peptide"
PEWQTPSFPKIHKLQE

SEQ ID NO: 70 - protein, 16 aa, synthetic construct
  REGION 1..16: note = source = /note="Description of Artificial Sequence: Synthetic peptide"
PSFPKIHKLQEDIVDR

SEQ ID NO: 71 - protein, 16 aa, synthetic construct
  REGION 1..16: note = source = /note="Description of Artificial Sequence: Synthetic peptide"
IHKLQEDIVDRCKQFV

SEQ ID NO: 72 - protein, 16 aa, synthetic construct
  REGION 1..16: note = source = /note="Description of Artificial Sequence: Synthetic peptide"
EDIVDRCKQFVGPLTV

SEQ ID NO: 73 - protein, 16 aa, synthetic construct
  REGION 1..16: note = source = /note="Description of Artificial Sequence: Synthetic peptide"
RCKQFVGPLTVNEKRR

SEQ ID NO: 74 - protein, 16 aa, synthetic construct
  REGION 1..16: note = source = /note="Description of Artificial Sequence: Synthetic peptide"
VGPLTVNEKRRLKLIM

SEQ ID NO: 75 - protein, 16 aa, synthetic construct
  REGION 1..16: note = source = /note="Description of Artificial Sequence: Synthetic peptide"
VNEKRRLKLIMPARFY

SEQ ID NO: 76 - protein, 16 aa, synthetic construct
  REGION 1..16: note = source = /note="Description of Artificial Sequence: Synthetic peptide"
RLKLIMPARFYPNVTK

SEQ ID NO: 77 - protein, 16 aa, synthetic construct
  REGION 1..16: note = source = /note="Description of Artificial Sequence: Synthetic peptide"
MPARFYPNVTKYLPLD

SEQ ID NO: 78 - protein, 16 aa, synthetic construct
  REGION 1..16: note = source = /note="Description of Artificial Sequence: Synthetic peptide"
YPNVTKYLPLDKGIKP

SEQ ID NO: 79 - protein, 16 aa, synthetic construct
  REGION 1..16: note = source = /note="Description of Artificial Sequence: Synthetic peptide"
KYLPLDKGIKPYYPEH

SEQ ID NO: 80 - protein, 16 aa, synthetic construct
  REGION 1..16: note = source = /note="Description of Artificial Sequence: Synthetic peptide"
DKGIKPYYPEHAVNHY

SEQ ID NO: 81 - protein, 16 aa, synthetic construct
  REGION 1..16: note = source = /note="Description of Artificial Sequence: Synthetic peptide"
PYYPEHAVNHYFQTRH

SEQ ID NO: 82 - protein, 16 aa, synthetic construct
  REGION 1..16: note = source = /note="Description of Artificial Sequence: Synthetic peptide"
HAVNHYFQTRHYLHTL

SEQ ID NO: 83 - protein, 16 aa, synthetic construct
  REGION 1..16: note = source = /note="Description of Artificial Sequence: Synthetic peptide"
YFQTRHYLHTLWKAGI

SEQ ID NO: 84 - protein, 16 aa, synthetic construct
  REGION 1..16: note = source = /note="Description of Artificial Sequence: Synthetic peptide"
HYLHTLWKAGILYKRE

SEQ ID NO: 85 - protein, 16 aa, synthetic construct
  REGION 1..16: note = source = /note="Description of Artificial Sequence: Synthetic peptide"
LWKAGILYKRETTRSA

SEQ ID NO: 86 - protein, 16 aa, synthetic construct
  REGION 1..16: note = source = /note="Description of Artificial Sequence: Synthetic peptide"
ILYKRETTRSASFCGS

SEQ ID NO: 87 - protein, 16 aa, synthetic construct
  REGION 1..16: note = source = /note="Description of Artificial Sequence: Synthetic peptide"
ETTRSASFCGSPYSWE

SEQ ID NO: 88 - protein, 16 aa, synthetic construct
  REGION 1..16: note = source = /note="Description of Artificial Sequence: Synthetic peptide"
ASFCGSPYSWEQELQH

SEQ ID NO: 89 - protein, 16 aa, synthetic construct
  REGION 1..16: note = source = /note="Description of Artificial Sequence: Synthetic peptide"
SPYSWEQELQHGSCWW

SEQ ID NO: 90 - protein, 16 aa, synthetic construct
  REGION 1..16: note = source = /note="Description of Artificial Sequence: Synthetic peptide"
EQELQHGSCWWLQFRN

SEQ ID NO: 91 - protein, 16 aa, synthetic construct
  REGION 1..16: note = source = /note="Description of Artificial Sequence: Synthetic peptide"
HGSCWWLQFRNSKPCS

SEQ ID NO: 92 - protein, 16 aa, synthetic construct
  REGION 1..16: note = source = /note="Description of Artificial Sequence: Synthetic peptide"
WLQFRNSKPCSEYCLT

SEQ ID NO: 93 - protein, 16 aa, synthetic construct
  REGION 1..16: note = source = /note="Description of Artificial Sequence: Synthetic peptide"
NSKPCSEYCLTHLVNL

SEQ ID NO: 94 - protein, 16 aa, synthetic construct
  REGION 1..16: note = source = /note="Description of Artificial Sequence: Synthetic peptide"
SEYCLTHLVNLLEDWG

SEQ ID NO: 95 - protein, 16 aa, synthetic construct
  REGION 1..16: note = source = /note="Description of Artificial Sequence: Synthetic peptide"
THLVNLLEDWGPCDEH

SEQ ID NO: 96 - protein, 16 aa, synthetic construct
  REGION 1..16: note = source = /note="Description of Artificial Sequence: Synthetic peptide"
LLEDWGPCDEHGEHHI

SEQ ID NO: 97 - protein, 16 aa, synthetic construct
  REGION 1..16: note = source = /note="Description of Artificial Sequence: Synthetic peptide"
GPCDEHGEHHIRIPRT

SEQ ID NO: 98 - protein, 16 aa, synthetic construct
  REGION 1..16: note = source = /note="Description of Artificial Sequence: Synthetic peptide"
HGEHHIRIPRTPARVT

SEQ ID NO: 99 - protein, 16 aa, synthetic construct
  REGION 1..16: note = source = /note="Description of Artificial Sequence: Synthetic peptide"
IRIPRTPARVTGGVFL

SEQ ID NO: 100 - protein, 16 aa, synthetic construct
  REGION 1..16: note = source = /note="Description of Artificial Sequence: Synthetic peptide"
TPARVTGGVFLVDKNP

SEQ ID NO: 101 - protein, 16 aa, synthetic construct
  REGION 1..16: note = source = /note="Description of Artificial Sequence: Synthetic peptide"
TGGVFLVDKNPHNTAE

SEQ ID NO: 102 - protein, 16 aa, synthetic construct
  REGION 1..16: note = source = /note="Description of Artificial Sequence: Synthetic peptide"
LVDKNPHNTAESRLVV

SEQ ID NO: 103 - protein, 16 aa, synthetic construct
  REGION 1..16: note = source = /note="Description of Artificial Sequence: Synthetic peptide"
PHNTAESRLVVDFSQF

SEQ ID NO: 104 - protein, 16 aa, synthetic construct
  REGION 1..16: note = source = /note="Description of Artificial Sequence: Synthetic peptide"
ESRLVVDFSQFSRGIT

SEQ ID NO: 105 - protein, 16 aa, synthetic construct
  REGION 1..16: note = source = /note="Description of Artificial Sequence: Synthetic peptide"
VDFSQFSRGITRVSWP

SEQ ID NO: 106 - protein, 16 aa, synthetic construct
  REGION 1..16: note = source = /note="Description of Artificial Sequence: Synthetic peptide"
FSRGITRVSWPKFAVP

SEQ ID NO: 107 - protein, 16 aa, synthetic construct
  REGION 1..16: note = source = /note="Description of Artificial Sequence: Synthetic peptide"
TRVSWPKFAVPNLQSL

SEQ ID NO: 108 - protein, 16 aa, synthetic construct
  REGION 1..16: note = source = /note="Description of Artificial Sequence: Synthetic peptide"
PKFAVPNLQSLTNLLS

SEQ ID NO: 109 - protein, 16 aa, synthetic construct
  REGION 1..16: note = source = /note="Description of Artificial Sequence: Synthetic peptide"
PNLQSLTNLLSSNLSW

SEQ ID NO: 110 - protein, 16 aa, synthetic construct
  REGION 1..16: note = source = /note="Description of Artificial Sequence: Synthetic peptide"
LTNLLSSNLSWLSLDV

SEQ ID NO: 111 - protein, 16 aa, synthetic construct
  REGION 1..16: note = source = /note="Description of Artificial Sequence: Synthetic peptide"
SSNLSWLSLDVSAAFY

SEQ ID NO: 112 - protein, 16 aa, synthetic construct
  REGION 1..16: note = source = /note="Description of Artificial Sequence: Synthetic peptide"
WLSLDVSAAFYHIPLH

SEQ ID NO: 113 - protein, 16 aa, synthetic construct
  REGION 1..16: note = source = /note="Description of Artificial Sequence: Synthetic peptide"
VSAAFYHIPLHPAAMP

SEQ ID NO: 114 - protein, 15 aa, synthetic construct
  REGION 1..15: note = source = /note="Description of Artificial Sequence: Synthetic peptide"
HLLVGSSGLSRYVAR

SEQ ID NO: 115 - protein, 16 aa, synthetic construct
  REGION 1..16: note = source = /note="Description of Artificial Sequence: Synthetic peptide"
SSGLSRYVARLSSNSR

SEQ ID NO: 116 - protein, 16 aa, synthetic construct
  REGION 1..16: note = source = /note="Description of Artificial Sequence: Synthetic peptide"
RYVARLSSNSRIINHQ

SEQ ID NO: 117 - protein, 16 aa, synthetic construct
  REGION 1..16: note = source = /note="Description of Artificial Sequence: Synthetic peptide"
LSSNSRIINHQHGTMQ

SEQ ID NO: 118 - protein, 16 aa, synthetic construct
  REGION 1..16: note = source = /note="Description of Artificial Sequence: Synthetic peptide"
RIINHQHGTMQNLHDS

SEQ ID NO: 119 - protein, 16 aa, synthetic construct
  REGION 1..16: note = source = /note="Description of Artificial Sequence: Synthetic peptide"
QHGTMQNLHDSCSRNL

SEQ ID NO: 120 - protein, 16 aa, synthetic construct
  REGION 1..16: note = source = /note="Description of Artificial Sequence: Synthetic peptide"
QNLHDSCSRNLYVSLL

SEQ ID NO: 121 - protein, 16 aa, synthetic construct
  REGION 1..16: note = source = /note="Description of Artificial Sequence: Synthetic peptide"
SCSRNLYVSLLLLYKT

SEQ ID NO: 122 - protein, 16 aa, synthetic construct
  REGION 1..16: note = source = /note="Description of Artificial Sequence: Synthetic peptide"
LYVSLLLLYKTFGRKL

SEQ ID NO: 123 - protein, 16 aa, synthetic construct
  REGION 1..16: note = source = /note="Description of Artificial Sequence: Synthetic peptide"
LLLYKTFGRKLHLYSH

SEQ ID NO: 124 - protein, 16 aa, synthetic construct
  REGION 1..16: note = source = /note="Description of Artificial Sequence: Synthetic peptide"
TFGRKLHLYSHPIILK

SEQ ID NO: 125 - protein, 16 aa, synthetic construct
  REGION 1..16: note = source = /note="Description of Artificial Sequence: Synthetic peptide"
LHLYSHPIILKTKRWG

SEQ ID NO: 126 - protein, 16 aa, synthetic construct
  REGION 1..16: note = source = /note="Description of Artificial Sequence: Synthetic peptide"
HPIILKTKRWGYSLNF

SEQ ID NO: 127 - protein, 16 aa, synthetic construct
  REGION 1..16: note = source = /note="Description of Artificial Sequence: Synthetic peptide"
KTKRWGYSLNFMGYVI

SEQ ID NO: 128 - protein, 16 aa, synthetic construct
  REGION 1..16: note = source = /note="Description of Artificial Sequence: Synthetic peptide"
GYSLNFMGYVIGSWGS

SEQ ID NO: 129 - protein, 16 aa, synthetic construct
  REGION 1..16: note = source = /note="Description of Artificial Sequence: Synthetic peptide"
FMGYVIGSWGSLPQDH

SEQ ID NO: 130 - protein, 16 aa, synthetic construct
  REGION 1..16: note = source = /note="Description of Artificial Sequence: Synthetic peptide"
IGSWGSLPQDHIIQKI

SEQ ID NO: 131 - protein, 16 aa, synthetic construct
  REGION 1..16: note = source = /note="Description of Artificial Sequence: Synthetic peptide"
SLPQDHIIQKIKECFR

SEQ ID NO: 132 - protein, 16 aa, synthetic construct
  REGION 1..16: note = source = /note="Description of Artificial Sequence: Synthetic peptide"
HIIQKIKECFRKLPVN

SEQ ID NO: 133 - protein, 16 aa, synthetic construct
  REGION 1..16: note = source = /note="Description of Artificial Sequence: Synthetic peptide"
IKECFRKLPVNRPIDW

SEQ ID NO: 134 - protein, 16 aa, synthetic construct
  REGION 1..16: note = source = /note="Description of Artificial Sequence: Synthetic peptide"
RKLPVNRPIDWKVCQR

SEQ ID NO: 135 - protein, 16 aa, synthetic construct
  REGION 1..16: note = source = /note="Description of Artificial Sequence: Synthetic peptide"
NRPIDWKVCQRIVGLL

SEQ ID NO: 136 - protein, 16 aa, synthetic construct
  REGION 1..16: note = source = /note="Description of Artificial Sequence: Synthetic peptide"
WKVCQRIVGLLGFAAP

SEQ ID NO: 137 - protein, 16 aa, synthetic construct
  REGION 1..16: note = source = /note="Description of Artificial Sequence: Synthetic peptide"
RIVGLLGFAAPFTQCG

SEQ ID NO: 138 - protein, 16 aa, synthetic construct
  REGION 1..16: note = source = /note="Description of Artificial Sequence: Synthetic peptide"
LGFAAPFTQCGYPALM

SEQ ID NO: 139 - protein, 16 aa, synthetic construct
  REGION 1..16: note = source = /note="Description of Artificial Sequence: Synthetic peptide"
PFTQCGYPALMPLYAC

SEQ ID NO: 140 - protein, 16 aa, synthetic construct
  REGION 1..16: note = source = /note="Description of Artificial Sequence: Synthetic peptide"
GYPALMPLYACIQSKQ

SEQ ID NO: 141 - protein, 16 aa, synthetic construct
  REGION 1..16: note = source = /note="Description of Artificial Sequence: Synthetic peptide"
MPLYACIQSKQAFTFS

SEQ ID NO: 142 - protein, 16 aa, synthetic construct
  REGION 1..16: note = source = /note="Description of Artificial Sequence: Synthetic peptide"
CIQSKQAFTFSPTYKA

SEQ ID NO: 143 - protein, 16 aa, synthetic construct
  REGION 1..16: note = source = /note="Description of Artificial Sequence: Synthetic peptide"
QAFTFSPTYKAFLSKQ

SEQ ID NO: 144 - protein, 16 aa, synthetic construct
  REGION 1..16: note = source = /note="Description of Artificial Sequence: Synthetic peptide"
SPTYKAFLSKQYLNLY

SEQ ID NO: 145 - protein, 16 aa, synthetic construct
  REGION 1..16: note = source = /note="Description of Artificial Sequence: Synthetic peptide"
AFLSKQYLNLYPVARQ

SEQ ID NO: 146 - protein, 16 aa, synthetic construct
  REGION 1..16: note = source = /note="Description of Artificial Sequence: Synthetic peptide"
QYLNLYPVARQRPGLC

SEQ ID NO: 147 - protein, 16 aa, synthetic construct
  REGION 1..16: note = source = /note="Description of Artificial Sequence: Synthetic peptide"
YPVARQRPGLCQVFAD

SEQ ID NO: 148 - protein, 16 aa, synthetic construct
  REGION 1..16: note = source = /note="Description of Artificial Sequence: Synthetic peptide"
QRPGLCQVFADATPTG

SEQ ID NO: 149 - protein, 16 aa, synthetic construct
  REGION 1..16: note = source = /note="Description of Artificial Sequence: Synthetic peptide"
CQVFADATPTGWGLAM

SEQ ID NO: 150 - protein, 16 aa, synthetic construct
  REGION 1..16: note = source = /note="Description of Artificial Sequence: Synthetic peptide"
DATPTGWGLAMGHQRM

SEQ ID NO: 151 - protein, 16 aa, synthetic construct
  REGION 1..16: note = source = /note="Description of Artificial Sequence: Synthetic peptide"
GWGLAMGHQRMRGTFV

SEQ ID NO: 152 - protein, 16 aa, synthetic construct
  REGION 1..16: note = source = /note="Description of Artificial Sequence: Synthetic peptide"
MGHQRMRGTFVAPLPI

SEQ ID NO: 153 - protein, 16 aa, synthetic construct
  REGION 1..16: note = source = /note="Description of Artificial Sequence: Synthetic peptide"
MRGTFVAPLPIHTAEL

SEQ ID NO: 154 - protein, 16 aa, synthetic construct
  REGION 1..16: note = source = /note="Description of Artificial Sequence: Synthetic peptide"
VAPLPIHTAELLAACF

SEQ ID NO: 155 - protein, 16 aa, synthetic construct
  REGION 1..16: note = source = /note="Description of Artificial Sequence: Synthetic peptide"
IHTAELLAACFARSRS

SEQ ID NO: 156 - protein, 16 aa, synthetic construct
  REGION 1..16: note = source = /note="Description of Artificial Sequence: Synthetic peptide"
LLAACFARSRSGAKIL

SEQ ID NO: 157 - protein, 16 aa, synthetic construct
  REGION 1..16: note = source = /note="Description of Artificial Sequence: Synthetic peptide"
FARSRSGAKILGTDNS

SEQ ID NO: 158 - protein, 16 aa, synthetic construct
  REGION 1..16: note = source = /note="Description of Artificial Sequence: Synthetic peptide"
SGAKILGTDNSVVLSR

SEQ ID NO: 159 - protein, 16 aa, synthetic construct
  REGION 1..16: note = source = /note="Description of Artificial Sequence: Synthetic peptide"
LGTDNSVVLSRKYTSF

SEQ ID NO: 160 - protein, 16 aa, synthetic construct
  REGION 1..16: note = source = /note="Description of Artificial Sequence: Synthetic peptide"
SVVLSRKYTSFPWLLG

SEQ ID NO: 161 - protein, 16 aa, synthetic construct
  REGION 1..16: note = source = /note="Description of Artificial Sequence: Synthetic peptide"
RKYTSFPWLLGCAANW

SEQ ID NO: 162 - protein, 16 aa, synthetic construct
  REGION 1..16: note = source = /note="Description of Artificial Sequence: Synthetic peptide"
FPWLLGCAANWILRGT

SEQ ID NO: 163 - protein, 16 aa, synthetic construct
  REGION 1..16: note = source = /note="Description of Artificial Sequence: Synthetic peptide"
GCAANWILRGTSFVYV

SEQ ID NO: 164 - protein, 16 aa, synthetic construct
  REGION 1..16: note = source = /note="Description of Artificial Sequence: Synthetic peptide"
WILRGTSFVYVPSALN

SEQ ID NO: 165 - protein, 16 aa, synthetic construct
  REGION 1..16: note = source = /note="Description of Artificial Sequence: Synthetic peptide"
TSFVYVPSALNPADDP

SEQ ID NO: 166 - protein, 16 aa, synthetic construct
  REGION 1..16: note = source = /note="Description of Artificial Sequence: Synthetic peptide"
VPSALNPADDPSRGRL

SEQ ID NO: 167 - protein, 16 aa, synthetic construct
  REGION 1..16: note = source = /note="Description of Artificial Sequence: Synthetic peptide"
NPADDPSRGRLGLSRP

SEQ ID NO: 168 - protein, 16 aa, synthetic construct
  REGION 1..16: note = source = /note="Description of Artificial Sequence: Synthetic peptide"
PSRGRLGLSRPLLRLP

SEQ ID NO: 169 - protein, 16 aa, synthetic construct
  REGION 1..16: note = source = /note="Description of Artificial Sequence: Synthetic peptide"
LGLSRPLLRLPFRPTT

SEQ ID NO: 170 - protein, 16 aa, synthetic construct
  REGION 1..16: note = source = /note="Description of Artificial Sequence: Synthetic peptide"
PLLRLPFRPTTGRTSL

SEQ ID NO: 171 - protein, 16 aa, synthetic construct
  REGION 1..16: note = source = /note="Description of Artificial Sequence: Synthetic peptide"
PFRPTTGRTSLYAVSP

SEQ ID NO: 172 - protein, 13 aa, synthetic construct
  REGION 1..13: note = source = /note="Description of Artificial Sequence: Synthetic peptide"
TGRTSLYAVSPSV

SEQ ID NO: 173 - protein, 200 aa, synthetic construct
  REGION 1..200: note = source = /note="Description of Artificial Sequence: Synthetic polypeptide"
HFRKLLLLDEEAGPLEEELPRLADEGLNRRVAEDLNLGNLPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVT

SEQ ID NO: 174 - protein, 380 aa, synthetic construct
  REGION 1..380: note = source = /note="Description of Artificial Sequence: Synthetic polypeptide"
YLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDVQAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADDVGSNLEDPASRELVVSYVNVNMGLKIRQLLWFHISCLTFGRETVIEYLVSFGVWIRTPPAYRPPNAPILSTLPETTVVRRRDRGR

SEQ ID NO: 175 - protein, 410 aa, synthetic construct
  REGION 1..410: note = source = /note="Description of Artificial Sequence: Synthetic polypeptide"
HFRKLLLLDEEAGPLEEELPRLADEGLNRRVAEDLNLGNLPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTQAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADDVGSNLEDPASRELVVSYVNVNMGLKIRQLLWFHISCLTFGRETVIEYLVSFGVWIRTPPAYRPPNAPILSTLPETTVVRRRDRGR

SEQ ID NO: 176 - DNA, 1140 bp, synthetic construct
  misc_feature 1..1140: note = source = /note="Description of Artificial Sequence: Synthetic polynucleotide"
  source 1..1140: mol_type = other DNA
tatctgccgctggataaaggcattaaaccgtattatccggaacatgcggtgaaccattattttcagacccgccattatctgcataccctgtggaaagcgggcattctgtataaacgcgaaaccacccgcagcgcgagcttttgcggcagcccgtatagctgggaacaggaactgcagcatggcagctgctggtggctgcagtttcgcaacagcaaaccgtgcagcgaatattgcctgacccatctggtgaacctgctggaagattggggaccgtgcgatgaacatggcgaacatcatattcgcattccgcgcaccccggcgcgcgtgaccggcggcgtgtttctggtggataaaaacccgcataacaccgcggaaagccgcctggtggtggattttagccagtttagccgcggcattacccgcgtgagctggccgaaatttgcggtgccgaacctgcagagcctgaccaacctgctgagcagcaacctgagctggctgagcctggatgtgcaggcgtttacctttagcccgacctataaagcgtttctgagcaaacagtatctgaacctgtatccggtggcgcgccagcgcccgggcctgtgccaggtgtttgcggatgcgaccccgaccggctggggcctggcgatgggccatcagcgcatgcgcggcacctttgtggcgccgctgccgattcataccgcggaactgctggcggcgtgctttgcgcgcagccgcagcggcgcgaaaattctgggcaccgataacagcgtggtgctgagccgcaaatataccagctttccgtggctgctgggctgcgcggcgaactggattctgcgcggcaccagctttgtgtatgtgccgagcgcgctgaacccggcggatgatgtgggcagcaacctggaagatccggcgagccgcgaactggtggtgagctatgtgaacgtgaacatgggcctgaaaattcgccagctgctgtggtttcatattagctgcctgacctttggccgcgaaaccgtgattgaatatctggtgagctttggcgtgtggattcgcaccccgccggcgtatcgcccgccgaacgcgccgattctgagcaccctgccggaaaccaccgtggtgcgccgccgcgatcggggccgc

SEQ ID NO: 177 - DNA, 1230 bp, synthetic construct
  misc_feature 1..1230: note = source = /note="Description of Artificial Sequence: Synthetic polynucleotide"
  source 1..1230: mol_type = other DNA
cattttcgcaaactgctgctgctggatgaagaagcgggaccgctggaagaagaactgccgcgcctggcggatgaaggcctgaaccgccgcgtggcggaagatctgaacctgggcaacctgccggaatggcagaccccgagctttccgaaaattcatctgcaggaagatattgtggatcgctgcaaacagtttgtgggaccgctgaccgtgaacgaaaaacgccgcctgaaactgattatgccggcgcgcttttatccgaacgtgaccaaatatctgccgctggataaaggcattaaaccgtattatccggaacatgcggtgaaccattattttcagacccgccattatctgcataccctgtggaaagcgggcattctgtataaacgcgaaaccacccgcagcgcgagcttttgcggcagcccgtatagctgggaacaggaactgcagcatggcagctgctggtggctgcagtttcgcaacagcaaaccgtgcagcgaatattgcctgacccatctggtgaacctgctggaagattggggaccgtgcgatgaacatggcgaacatcatattcgcattccgcgcaccccggcgcgcgtgacccaggcgtttacctttagcccgacctataaagcgtttctgagcaaacagtatctgaacctgtatccggtggcgcgccagcgcccgggcctgtgccaggtgtttgcggatgcgaccccgaccggctggggcctggcgatgggccatcagcgcatgcgcggcacctttgtggcgccgctgccgattcataccgcggaactgctggcggcgtgctttgcgcgcagccgcagcggcgcgaaaattctgggcaccgataacagcgtggtgctgagccgcaaatataccagctttccgtggctgctgggctgcgcggcgaactggattctgcgcggcaccagctttgtgtatgtgccgagcgcgctgaacccggcggatgatgtgggcagcaacctggaagatccggcgagccgcgaactggtggtgagctatgtgaacgtgaacatgggcctgaaaattcgccagctgctgtggtttcatattagctgcctgacctttggccgcgaaaccgtgattgaatatctggtgagctttggcgtgtggattcgcaccccgccggcgtatcgcccgccgaacgcgccgattctgagcaccctgccggaaaccaccgtggtgcgccgccgagatcgaggccgc

SEQ ID NO: 178 - protein, 170 aa, synthetic construct
  REGION 1..170: note = source = /note="Description of Artificial Sequence: Synthetic polypeptide"
YLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDV

SEQ ID NO: 179 - protein, 125 aa, synthetic construct
  REGION 1..125: note = source = /note="Description of Artificial Sequence: Synthetic polypeptide"
QAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADD

SEQ ID NO: 180 - protein, 85 aa, synthetic construct
  REGION 1..85: note = source = /note="Description of Artificial Sequence: Synthetic polypeptide"
VGSNLEDPASRELVVSYVNVNMGLKIRQLLWFHISCLTFGRETVIEYLVSFGVWIRTPPAYRPPNAPILSTLPETTVVRRRDRGR

SEQ ID NO: 181 - protein, 200 aa, synthetic construct; identical to SEQ ID NO: 173
  REGION 1..200: note = source = /note="Description of Artificial Sequence: Synthetic polypeptide"

SEQ ID NO: 182 - protein, 125 aa, synthetic construct; identical to SEQ ID NO: 179
  REGION 1..125: note = source = /note="Description of Artificial Sequence: Synthetic polypeptide"

SEQ ID NO: 183 - protein, 85 aa, synthetic construct; identical to SEQ ID NO: 180
  REGION 1..85: note = source = /note="Description of Artificial Sequence: Synthetic polypeptide"

SEQ ID NO: 184 - DNA, 2331 bp, synthetic construct
  misc_feature 1..2331: note = source = /note="Description of Artificial Sequence: Synthetic polynucleotide"
  source 1..2331: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccctatctgccgctggataaaggcattaaaccgtattatccggaacatgcggtgaaccattattttcagacccgccattatctgcataccctgtggaaagcgggcattctgtataaacgcgaaaccacccgcagcgcgagcttttgcggcagcccgtatagctgggaacaggaactgcagcatggcagctgctggtggctgcagtttcgcaacagcaaaccgtgcagcgaatattgcctgacccatctggtgaacctgctggaagattggggaccgtgcgatgaacatggcgaacatcatattcgcattccgcgcaccccggcgcgcgtgaccggcggcgtgtttctggtggataaaaacccgcataacaccgcggaaagccgcctggtggtggattttagccagtttagccgcggcattacccgcgtgagctggccgaaatttgcggtgccgaacctgcagagcctgaccaacctgctgagcagcaacctgagctggctgagcctggatgtgcaggcgtttacctttagcccgacctataaagcgtttctgagcaaacagtatctgaacctgtatccggtggcgcgccagcgcccgggcctgtgccaggtgtttgcggatgcgaccccgaccggctggggcctggcgatgggccatcagcgcatgcgcggcacctttgtggcgccgctgccgattcataccgcggaactgctggcggcgtgctttgcgcgcagccgcagcggcgcgaaaattctgggcaccgataacagcgtggtgctgagccgcaaatataccagctttccgtggctgctgggctgcgcggcgaactggattctgcgcggcaccagctttgtgtatgtgccgagcgcgctgaacccggcggatgatgtgggcagcaacctggaagatccggcgagccgcgaactggtggtgagctatgtgaacgtgaacatgggcctgaaaattcgccagctgctgtggtttcatattagctgcctgacctttggccgcgaaaccgtgattgaatatctggtgagctttggcgtgtggattcgcaccccgccggcgtatcgcccgccgaacgcgccgattctgagcaccctgccggaaaccaccgtggtgcgccgccgcgatcggggccgcgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttactag

SEQ ID NO: 185 - protein, 776 aa, synthetic construct
  REGION 1..776: note = source = /note="Description of Artificial Sequence: Synthetic polypeptide"
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDVQAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADDVGSNLEDPASRELVVSYVNVNMGLKIRQLLWFHISCLTFGRETVIEYLVSFGVWIRTPPAYRPPNAPILSTLPETTVVRRRDRGRGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 186 - DNA, 2421 bp, synthetic construct
  misc_feature 1..2421: note = source = /note="Description of Artificial Sequence: Synthetic polynucleotide"
  source 1..2421: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccattttcgcaaactgctgctgctggatgaagaagcgggaccgctggaagaagaactgccgcgcctggcggatgaaggcctgaaccgccgcgtggcggaagatctgaacctgggcaacctgccggaatggcagaccccgagctttccgaaaattcatctgcaggaagatattgtggatcgctgcaaacagtttgtgggaccgctgaccgtgaacgaaaaacgccgcctgaaactgattatgccggcgcgcttttatccgaacgtgaccaaatatctgccgctggataaaggcattaaaccgtattatccggaacatgcggtgaaccattattttcagacccgccattatctgcataccctgtggaaagcgggcattctgtataaacgcgaaaccacccgcagcgcgagcttttgcggcagcccgtatagctgggaacaggaactgcagcatggcagctgctggtggctgcagtttcgcaacagcaaaccgtgcagcgaatattgcctgacccatctggtgaacctgctggaagattggggaccgtgcgatgaacatggcgaacatcatattcgcattccgcgcaccccggcgcgcgtgacccaggcgtttacctttagcccgacctataaagcgtttctgagcaaacagtatctgaacctgtatccggtggcgcgccagcgcccgggcctgtgccaggtgtttgcggatgcgaccccgaccggctggggcctggcgatgggccatcagcgcatgcgcggcacctttgtggcgccgctgccgattcataccgcggaactgctggcggcgtgctttgcgcgcagccgcagcggcgcgaaaattctgggcaccgataacagcgtggtgctgagccgcaaatataccagctttccgtggctgctgggctgcgcggcgaactggattctgcgcggcaccagctttgtgtatgtgccgagcgcgctgaacccggcggatgatgtgggcagcaacctggaagatccggcgagccgcgaactggtggtgagctatgtgaacgtgaacatgggcctgaaaattcgccagctgctgtggtttcatattagctgcctgacctttggccgcgaaaccgtgattgaatatctggtgagctttggcgtgtggattcgcaccccgccggcgtatcgcccgccgaacgcgccgattctgagcaccctgccggaaaccaccgtggtgcgccgccgagatcgaggccgcgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttactag

SEQ ID NO: 187 - protein, 806 aa, synthetic construct
  REGION 1..806: note = source = /note="Description of Artificial Sequence: Synthetic polypeptide"
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHFRKLLLLDEEAGPLEEELPRLADEGLNRRVAEDLNLGNLPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTQAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADDVGSNLEDPASRELVVSYVNVNMGLKIRQLLWFHISCLTFGRETVIEYLVSFGVWIRTPPAYRPPNAPILSTLPETTVVRRRDRGRGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 188 - protein, 9 aa, synthetic construct
  REGION 1..9: note = source = /note="Description of Artificial Sequence: Synthetic peptide"
FAVPNLQSL

SEQ ID NO: 189 - protein, 15 aa, synthetic construct
  REGION 1..15: note = source = /note="Description of Artificial Sequence: Synthetic peptide"
PEWQTPSFPKIHLQE

SEQ ID NO: 190 - protein, 15 aa, synthetic construct
  REGION 1..15: note = source = /note="Description of Artificial Sequence: Synthetic peptide"
PSFPKIHLQEDIVDR

SEQ ID NO: 191 - protein, 15 aa, synthetic construct
  REGION 1..15: note = source = /note="Description of Artificial Sequence: Synthetic peptide"
IHLQEDIVDRCKQFV

SEQ ID NO: 192 - protein, 15 aa, synthetic construct
  REGION 1..15: note = source = /note="Description of Artificial Sequence: Synthetic peptide"
DIVDRCKQFVGPLTV

SEQ ID NO: 193 - protein, 15 aa, synthetic construct
  REGION 1..15: note = source = /note="Description of Artificial Sequence: Synthetic peptide"
CKQFVGPLTVNEKRR

SEQ ID NO: 194 - protein, 15 aa, synthetic construct
  REGION 1..15: note = source = /note="Description of Artificial Sequence: Synthetic peptide"
GPLTVNEKRRLKLIM

SEQ ID NO: 195 - protein, 15 aa, synthetic construct
  REGION 1..15: note = source = /note="Description of Artificial Sequence: Synthetic peptide"
NEKRRLKLIMPARFY

SEQ ID NO: 196 - protein, 15 aa, synthetic construct
  REGION 1..15: note = source = /note="Description of Artificial Sequence: Synthetic peptide"
LKLIMPARFYPNVTK

SEQ ID NO: 197 - protein, 15 aa, synthetic construct
  REGION 1..15: note = source = /note="Description of Artificial Sequence: Synthetic peptide"
PARFYPNVTKYLPLD

SEQ ID NO: 198 - protein, 15 aa, synthetic construct
  REGION 1..15: note = source = /note="Description of Artificial Sequence: Synthetic peptide"
PNVTKYLPLDKGIKP

SEQ ID NO: 199 - protein, 15 aa, synthetic construct
  REGION 1..15: note = source = /note="Description of Artificial Sequence: Synthetic peptide"
YLPLDKGIKPYYPEH

SEQ ID NO: 200 - protein, 15 aa, synthetic construct
  REGION 1..15: note = source = /note="Description of Artificial Sequence: Synthetic peptide"
KGIKPYYPEHAVNHY

SEQ ID NO: 201 - protein, 15 aa, synthetic construct
  REGION 1..15: note = source = /note="Description of Artificial Sequence: Synthetic peptide"
YYPEHAVNHYFQTRH

SEQ ID NO: 202 - protein, 15 aa, synthetic construct
  REGION 1..15: note = source = /note="Description of Artificial Sequence: Synthetic peptide"
AVNHYFQTRHYLHTL

SEQ ID NO: 203 - protein, 15 aa, synthetic construct
  REGION 1..15: note = source = /note="Description of Artificial Sequence: Synthetic peptide"
FQTRHYLHTLWKAGI

SEQ ID NO: 204 - protein, 15 aa, synthetic construct
  REGION 1..15: note = source = /note="Description of Artificial Sequence: Synthetic peptide"
YLHTLWKAGILYKRE

SEQ ID NO: 205 - protein, 15 aa, synthetic construct
  REGION 1..15: note = source = /note="Description of Artificial Sequence: Synthetic peptide"
WKAGILYKRETTRSA

SEQ ID NO: 206 - protein, 15 aa, synthetic construct
  REGION 1..15: note = source = /note="Description of Artificial Sequence: Synthetic peptide"
LYKRETTRSASFCGS

SEQ ID NO: 207 - protein, 15 aa, synthetic construct
  REGION 1..15: note = source = /note="Description of Artificial Sequence: Synthetic peptide"
TTRSASFCGSPYSWE

SEQ ID NO: 208 - protein, 15 aa, synthetic construct
  REGION 1..15: note = source = /note="Description of Artificial Sequence: Synthetic peptide"
SFCGSPYSWEQELQH

SEQ ID NO: 209 - protein, 15 aa, synthetic construct
  REGION 1..15: note = source = /note="Description of Artificial Sequence: Synthetic peptide"
PYSWEQELQHGSCWW

SEQ ID NO: 210 - protein, 15 aa, synthetic construct
  REGION 1..15: note = source = /note="Description of Artificial Sequence: Synthetic peptide"
QELQHGSCWWLQFRN

SEQ ID NO: 211 - protein, 15 aa, synthetic construct
  REGION 1..15: note = source = /note="Description of Artificial Sequence: Synthetic peptide"
GSCWWLQFRNSKPCS

SEQ ID NO: 212 - protein, 15 aa, synthetic construct
  REGION 1..15: note = source = /note="Description of Artificial Sequence: Synthetic peptide"
LQFRNSKPCSEYCLT

SEQ ID NO: 213 - protein, 15 aa, synthetic construct
  REGION 1..15: note = source = /note="Description of Artificial Sequence: Synthetic peptide"
SKPCSEYCLTHLVNL

SEQ ID NO: 214 - protein, 15 aa, synthetic construct
  REGION 1..15: note = source = /note="Description of Artificial Sequence: Synthetic peptide"
EYCLTHLVNLLEDWG

SEQ ID NO: 215 - protein, 15 aa, synthetic construct
  REGION 1..15: note = source = /note="Description of Artificial Sequence: Synthetic peptide"
HLVNLLEDWGPCDEH

SEQ ID NO: 216 - protein, 15 aa, synthetic construct
  REGION 1..15: note = source = /note="Description of Artificial Sequence: Synthetic peptide"
LEDWGPCDEHGEHHI

SEQ ID NO: 217 - protein, 15 aa, synthetic construct
  REGION 1..15: note = source = /note="Description of Artificial Sequence: Synthetic peptide"
PCDEHGEHHIRIPRT

SEQ ID NO: 218 - protein, 15 aa, synthetic construct
  REGION 1..15: note = source = /note="Description of Artificial Sequence: Synthetic peptide"
GEHHIRIPRTPARVT

SEQ ID NO: 219 - protein, 15 aa, synthetic construct
  REGION 1..15: note = source = /note="Description of Artificial Sequence: Synthetic peptide"
RIPRTPARVTGGVFL

SEQ ID NO: 220 - protein, 15 aa, synthetic construct
  REGION 1..15: note = source = /note="Description of Artificial Sequence: Synthetic peptide"
PARVTGGVFLVDKNP

SEQ ID NO: 221 - protein, 15 aa, synthetic construct
  REGION 1..15: note = source = /note="Description of Artificial Sequence: Synthetic peptide"
GGVFLVDKNPHNTAE

SEQ ID NO: 222 - protein, 15 aa, synthetic construct
  REGION 1..15: note = source = /note="Description of Artificial Sequence: Synthetic peptide"
VDKNPHNTAESRLVV

SEQ ID NO: 223 - protein, 15 aa, synthetic construct
  REGION 1..15: note = source = /note="Description of Artificial Sequence: Synthetic peptide"
HNTAESRLVVDFSQF

SEQ ID NO: 224 - protein, 15 aa, synthetic construct
  REGION 1..15: note = source = /note="Description of Artificial Sequence: Synthetic peptide"
SRLVVDFSQFSRGIT

SEQ ID NO: 225 - protein, 15 aa, synthetic construct
  REGION 1..15: note = source = /note="Description of Artificial Sequence: Synthetic peptide"
DFSQFSRGITRVSWP

SEQ ID NO: 226 - protein, 15 aa, synthetic construct
  REGION 1..15: note = source = /note="Description of Artificial Sequence: Synthetic peptide"
SRGITRVSWPKFAVP

SEQ ID NO: 227 - protein, 15 aa, synthetic construct
  REGION 1..15: note = source = /note="Description of Artificial Sequence: Synthetic peptide"
RVSWPKFAVPNLQSL

SEQ ID NO: 228 - protein, 15 aa, synthetic construct
  REGION 1..15: note = source = /note="Description of Artificial Sequence: Synthetic peptide"
KFAVPNLQSLTNLLS

SEQ ID NO: 229 - protein, 15 aa, synthetic construct
  REGION 1..15: note = source = /note="Description of Artificial Sequence: Synthetic peptide"
NLQSLTNLLSSNLSW

SEQ ID NO: 230 - protein, 15 aa, synthetic construct
  REGION 1..15: note = source = /note="Description of Artificial Sequence: Synthetic peptide"
TNLLSSNLSWLSLDV

SEQ ID NO: 231 - protein, 15 aa, synthetic construct
  REGION 1..15: note = source = /note="Description of Artificial Sequence: Synthetic peptide"
SNLSWLSLDVSAAFY

SEQ ID NO: 232 - protein, 15 aa, synthetic construct
  REGION 1..15: note = source = /note="Description of Artificial Sequence: Synthetic peptide"
LSLDVSAAFYHIPLH

SEQ ID NO: 233 - protein, 15 aa, synthetic construct
  REGION 1..15: note = source = /note="Description of Artificial Sequence: Synthetic peptide"
SAAFYHIPLHPAAMP

(19) United States
(12) Patent Application Publication
(10) Pub. No.: US 2024/0091347 A1
(43) Pub. Date: Mar. 21, 2024

(54) ADENOVIRAL VECTORS ENCODING HEPATITIS B VIRAL ANTIGENS FUSED TO HERPES VIRUS GLYCOPROTEIN D AND METHODS OF USING THE SAME

(71) Applicants: Virion Therapeutics, LLC, Newark, DE (US); The Wistar Institute, Philadelphia, PA (US)

(72) Inventors: Hildegund CJ ERTL, Villanova, PA (US); Colin Stephen MAGOWAN, Bishop, CA (US)

(21) Appl. No.: 18/492,945

(22) Filed: Oct. 24, 2023

Related U.S. Application Data
(63) Continuation of application No. 17/679,356, filed on Feb. 24, 2022, now Pat. No. 11,850,282.
Application No. 17/679,356 is a continuation of application No. 17/459,313, filed on Aug. 27, 2021, now Pat. No. 11,291,716.
Application No. 17/459,313 is a continuation of application No. PCT/US2021/012630, filed on Jan. 8, 2021.
(60) Provisional application No. 62/958,809, filed on Jan. 9, 2020.
(60) Provisional application No. 62/958,827, filed on Jan. 9, 2020.
(60) Provisional application No. 62/967,104, filed on Jan. 29, 2020.
(60) Provisional application No. 62/967,242, filed on Jan. 29, 2020.
(60) Provisional application No. 63/064,506, filed on Aug. 12, 2020.
(60) Provisional application No. 63/064,571, filed on Aug. 12, 2020.
(60) Provisional application No. 63/112,202, filed on Nov. 11, 2020.
(60) Provisional application No. 63/112,219, filed on Nov. 11, 2020.

(51) Int. Cl. A61K 39/29; A61P 31/12; C12N 7/00; C12N 15/86

(52) U.S. Cl. CPC A61K 39/292; A61P 31/12; C12N 7/00; C12N 15/86; A61K 2039/53

(57) ABSTRACT

Provided herein are non-naturally occurring variants of the hepatitis B virus (HBV) Core protein, the HBV polymerase N-terminal domain, and the HBV polymerase C-terminal domain, as well as immunogenic fragments thereof. Fusion proteins comprising the HBV variants fused to a herpes simplex virus (HSV) glycoprotein (gD) sequence, as well as methods of using the fusion proteins, are also provided.

### CROSS REFERENCE TO RELATED APPLICATIONS

This application is a continuation of U.S. patent application Ser. No. 17/679,356 filed on Feb. 24, 2022, which is a continuation of U.S. patent application Ser. No. 17/459,313, filed on Aug. 27, 2021 (U.S. Pat. No. 11,291,716), which is a continuation of International Application No. PCT/US2021/012630, filed on Jan. 8, 2021, which claims priority to U.S. Provisional Application No. 62/958,809, filed on Jan. 9, 2020, U.S. Provisional Application No. 62/958,827, filed on Jan. 9, 2020, U.S. Provisional Application No. 62/967,242, filed on Jan. 29, 2020, U.S. Provisional Application No. 62/967,104, filed on Jan. 29, 2020, U.S. Provisional Application No. 63/064,506, filed on Aug. 12, 2020, U.S. Provisional Application No. 63/064,571, filed on Aug. 12, 2020, U.S. Provisional Application No. 63/112,202, filed on Nov. 11, 2020, and U.S. Provisional Application No. 63/112,219, filed on Nov. 11, 2020, the disclosure of each of which is hereby incorporated by reference in their entirety.

### SEQUENCE LISTING

The instant application contains a Sequence Listing which has been submitted electronically in XML format and is hereby incorporated by reference in its entirety. The XML copy, created on Nov. 7, 2023, is named 111876_000083_SL_XML.txt and is 326 KB in size.

### FIELD OF THE INVENTION

Disclosed herein are non-naturally occurring variants of the hepatitis B virus (HBV) Core protein, the HBV polymerase N-terminal domain, and the HBV polymerase C-terminal domain, as well as immunogenic fragments thereof and fusion proteins comprising the same.

### BACKGROUND OF THE INVENTION

The World Health Organization estimates that, in 2015, 257 million people were living with chronic hepatitis B infection (defined as hepatitis B surface antigen positive) and that hepatitis B resulted in an estimated 887,000 deaths, mostly from cirrhosis and hepatocellular carcinoma (i.e., primary liver cancer). Assuming that women of reproductive age constitute 25.3% of the world's population (United Nations data), adults chronically infected may include 65 million women of childbearing age who can potentially transmit HBV to their babies (WHO Global Hepatitis Report 2017. Available at: apps_who_int/iris/bitstream/handle/10665/255016/9789241565455-eng.pdf;jsessionid=D78616700ED7322D4109CA4541FB94EA?sequence=1). The overall incidence rate in 2016 was 1.0 case per 100,000 population (Centers for Disease Control and Prevention. Viral Hepatitis Surveillance—United States, 2017. Atlanta: US Department of Health and Human Services, Centers for Disease Control and Prevention; 2019. Available at: www cdc gov/hepatitis/statistics/2017surveillance/index.htm.). In 2017 alone, a total of 3,407 cases of acute hepatitis B were reported to the Centers for Disease Control and Prevention (CDC).

Despite the availability of a prophylactic HBV vaccine, the burden of chronic HBV infection continues to be a significant unmet worldwide medical problem, due to suboptimal treatment options and sustained rates of new infections in most parts of the developing world.

### SUMMARY OF THE INVENTION

Provided herein is a hepatitis B virus (HBV) Core protein comprising the amino acid sequence of SEQ ID NO: 6 or an immunogenic fragment thereof

Also provided is an HBV polymerase N-terminal domain comprising the amino acid sequence of SEQ ID NO: 8 or an immunogenic fragment thereof

An HBV polymerase C-terminal domain comprising the amino acid sequence of SEQ ID NO: 10 or an immunogenic fragment thereof is also disclosed.

Fusion proteins comprising: an N-terminal herpes simplex virus (HSV) glycoprotein (gD) sequence or a variant thereof; the disclosed HBV Core protein, HBV polymerase N-terminal domain, HBV polymerase C-terminal domain, or immunogenic fragments thereof; and a C-terminal HSV gD sequence or a variant thereof are also provided.

Also provided herein are fusion proteins comprising: an N-terminal herpes simplex virus (HSV) glycoprotein (gD) sequence or a variant thereof; combinations of the disclosed HBV Core protein, HBV polymerase N-terminal domain, HBV polymerase C-terminal domain, and/or immunogenic fragments thereof; and a C-terminal HSV gD sequence or a variant thereof

Nucleic acid molecules encoding the disclosed proteins or fusion proteins, vectors comprising the nucleic acid molecules, and vaccines comprising the disclosed vectors are disclosed herein.

Also provided herein are methods of inducing an immune response to HBV in a subject, the method comprising providing to the subject an effective amount of any of the disclosed fusion proteins, nucleic acid molecules, vectors, or vaccines to thereby induce an immune response to HBV.

### BRIEF DESCRIPTION OF THE DRAWINGS

The summary, as well as the following detailed description, is further understood when read in conjunction with the appended drawings. For the purpose of illustrating the disclosed proteins, vaccines, and methods, there are shown in the drawings exemplary embodiments of the proteins, vaccines, and methods; however, the proteins, vaccines, and methods are not limited to the specific embodiments disclosed. In the drawings:

FIG. 1 illustrates the frequency of epitope-optimized Core amino acids. Amino acid residues are indicated on the X-axis; percent sequence similarity across all genomes analyzed is indicated on the Y-axis.

FIG. 2A, FIG. 2B, FIG. 2C, FIG. 2D, FIG. 2E, and FIG. 2F illustrate vaccine insert-specific T cell frequencies in C57B1/6 mice following intramuscular (i.m.) injection with the indicated doses of: replication-defective adenovirus vector of chimpanzee serotype 6 (AdC6) containing the epitope-optimized Core sequence genetically fused into gD (SEQ ID NO: 15) (AdC6-gDCore) (FIG. 2A and FIG. 2D); AdC6 containing the epitope-optimized polymerase C-terminal domain sequence genetically fused into gD (SEQ ID NO: 19) (AdC6-gDPolC) (FIG. 2B and FIG. 2E); and AdC6 containing the epitope-optimized polymerase N-terminal domain sequence genetically fused into gD (SEQ ID NO: 17) (AdC6-gDPolN) (FIG. 2C and FIG. 2F). Mice were bled 14 days after the injection and T cell frequencies to the various HBV inserts were analyzed by intracellular cytokine staining (ICS) for interferon (IFN)-γ upon stimulation of cells with overlapping peptides representing the HBV sequences. Control cells were cultured without peptides. Graphs show results for individual mice with medians indicated by the lines. FIG. 2A-2C show insert-specific CD8+ T cell frequency; FIG. 2D-2F show insert-specific CD4+ T cell frequency.

FIG. 3A, FIG. 3B, and FIG. 3C illustrate T cell frequencies in different mouse strains (A: C57B1/6 mice; B: BALB/c mice; C: HLA-A2 transgenic (tg) mice) to pools of peptides representing the indicated HBV sequence. Results were obtained with splenocytes harvested 4 weeks after immunization and tested by ICS for IFN-γ. Peptides were arranged in matrices so that recognition of 2 pools identified one peptide. The graphs show responses to the different pools; responses to a pool containing all peptides are shown to the right. Background frequencies obtained without the peptides were subtracted. Pools that were deemed to elicit a response and peptides identified in response to different pools are listed at the bottom of each figure. CD8+ T cell and CD4+ T cell responses are shown for BALB/c mice; CD8+ T cell responses are shown for HLA-A2 tg mice, which carry a human MHC class I molecule but mouse MHC class II molecules. T cells were gated on activated CD44+ cells. Each consecutively numbered “peptide” consists of 15 amino acids beginning on the 1^{st}, 6^{th}, 11^{th}, etc. amino acid of the Core, PolN, or PolC sequence. Thus, for example, peptide 1 of Core corresponds to amino acids 1-15 of SEQ ID NO: 6 (i.e. the epitope-optimized Core amino acid sequence), peptide 2 of Core corresponds to amino acids 6-20 of SEQ ID NO: 6, peptide 3 of Core corresponds to amino acids 11-25 of SEQ ID NO: 6, etc. Similarly, peptide 1 of PolN corresponds to amino acids 1-15 of SEQ ID NO: 8 (i.e. the epitope-optimized PolN amino acid sequence), peptide 2 of PolN corresponds to amino acids 6-20 of SEQ ID NO: 8, peptide 3 of PolN corresponds to amino acids 11-25 of SEQ ID NO: 8, etc. Likewise, peptide 1 of PolC corresponds to amino acids 1-15 of SEQ ID NO: 10 (i.e. the epitope-optimized PolC amino acid sequence), peptide 2 of PolC corresponds to amino acids 6-20 of SEQ ID NO: 10, peptide 3 of PolC corresponds to amino acids 11-25 of SEQ ID NO: 10, etc.

FIG. 4A, FIG. 4B and FIG. 4C show the IFN-γ response upon boosting with AdC6-gDCore (A), AdC6-gDPolC (B), and AdC6-gDPolN (C) in C57B1/6 mice immunized with various doses of the indicated vectors. The left graphs show responses tested from blood 2 weeks after priming with AdC6 vector. Mice were boosted 8 weeks later with the same doses of AdC7 vectors expressing the same inserts. The right graphs show responses at 2 weeks after the boost in blood.

FIG. 5A, FIG. 5B, and FIG. 5C illustrate T cell frequencies in different mouse strains (A: C57B1/6 mice; B: BALB/c mice; C: HLA-A2 tg mice) to pools of peptides representing the indicated HBV sequence. Mice were primed with AdC6 vectors expressing either of the 3 inserts (i.e., Core, PolC, or PolN) and were boosted 8 weeks later with AdC7 vectors expressing the same inserts. Results were obtained with splenocytes harvested 4 weeks after the immunization and tested by ICS for IFN-γ. Peptides were arranged in matrices so that recognition of 2 pools identified one peptide. The graphs show responses to the different pools; responses to a pool containing all peptides are shown to the right. Background frequencies obtained without the peptides were subtracted. Pools that were deemed to elicit a response and peptides identified in response to different pools are listed at the bottom of each figure. CD8+ T cell and CD4+ T cell responses are shown for BALB/c mice; CD8+ T cell responses are shown for HLA-A2 tg mice which carry a human MHC class I molecule but mouse MHC class II molecules. T cells were gated on activated CD44+ cells. Each consecutively numbered “peptide” consists of 15 amino acids beginning on the 1^{st}, 6^{th}, 11^{th}, etc. amino acid of the Core,

PolN, or PolC sequence. Thus, for example, peptide 1 of Core corresponds to amino acids 1-15 of SEQ ID NO: 6 (i.e. the epitope-optimized Core amino acid sequence), peptide 2 of Core corresponds to amino acids 6-20 of SEQ ID NO: 6, peptide 3 of Core corresponds to amino acids 11-25 of SEQ ID NO: 6, etc. Similarly, peptide 1 of PolN corresponds to amino acids 1-15 of SEQ ID NO: 8 (i.e. the epitope-optimized PolN amino acid sequence), peptide 2 of PolN corresponds to amino acids 6-20 of SEQ ID NO: 8, peptide 3 of PolN corresponds to amino acids 11-25 of SEQ ID NO: 8, etc. Likewise, peptide 1 of PolC corresponds to amino acids 1-15 of SEQ ID NO: 10 (i.e. the epitope-optimized PolC amino acid sequence), peptide 2 of PolC corresponds to amino acids 6-20 of SEQ ID NO: 10, peptide 3 of PolC corresponds to amino acids 11-25 of SEQ ID NO: 10, etc.

FIG. 6 illustrates the effect of vaccination on HBV genome copy numbers in serum upon AAV-1.3HBV challenge. A group of 3 mice were challenged with 1×10¹⁰, 1×10¹¹ or 1.5×10¹¹ virus genomes (vg) of an adeno-associated virus 8 (AAV8)-1.3HBV vector and 8 weeks later were vaccinated with AdC6-gDPolN. Viral titers were tested 8 weeks after vaccination and compared to pre-vaccination titers. Viral changes from baseline for each treatment group are shown.

FIG. 7A, FIG. 7B, FIG. 7C, FIG. 7D, and FIG. 7E illustrate exemplary HBV epitope shifting experiments. FIG. 7A—mice were immunized with the AdC6-gDPolN vaccine. Four weeks later splenocytes were tested by intracellular cytokine staining for IFN-γ responses to peptide pools representing the PolN sequence. T cells after stimulation were stained for T cell markers. FIG. 7B—results obtained with the same assay using splenocytes from mice that were challenged with 1×10¹⁰ vg of AAV8-1.3-HBV. Mice were vaccinated 4 weeks later and T cell responses were tested from spleens 10 weeks later. FIG. 7C—results obtained with the same assay using splenocytes from mice that had been challenged with 1.5×10¹¹ vg of AAV8-1.3-HBV. Mice were vaccinated 4 weeks later and T cell responses were tested from spleen 10 weeks later. FIG. 7A, 7B, and 7C show the frequencies of IFN-γ producing CD44+ CD8+ T cells over all CD44+ CD8+ T cells. Background responses obtained by splenocytes incubated without peptide pools were subtracted. FIG. 7D—peptide pools. FIG. 7E—individual peptide sequences. FIG. 7E discloses SEQ ID NOs: 55-68 and 189-233, respectively, in order of appearance.

FIG. 8A, FIG. 8B, and FIG. 8C show data from the same experiment described above in FIG. 7. Based on the responses to the peptide pools, it was determined which individual peptides (both pools and peptides shown in FIG. 7) were positive. The graphs show responses to all of the peptides. Each peptide was present in two pools and therefore two values for frequencies were obtained for each peptide; only the lower data points are shown in this figure.

FIG. 9A, FIG. 9B, and FIG. 9C illustrate the results from exemplary immunogenicity experiments performed on C57B1/6 mice (n=5 per group) injected with various doses of exemplary AdC6-gDCore, AdC6-gDPolN, or AdC6-gDPolC vectors and boosted with AdC7 vectors containing the same insert (i.e. AdC7-gDCore, AdC7-gDPolN, or AdC7-gDPolC vectors) two months after the first injection. FIG. 9A illustrates antigen immunogenicity, FIG. 9B illustrates the duration of response, and FIG. 9C illustrates the prime-boost response.

FIG. 10 illustrates the CD8+ T cell peptide recognition of PolN epitopes in BALB/c, C57B1/6, and HLA-A2 transgenic mice after vaccination with a prime of AdC6-gDPolN and a boost of AdC7-gDPolN. CD8+ T cell peptide recognition was calculated as the fraction of positive peptides recognized two weeks after either the prime or the boost by the total number of overlapping 8 peptides from PolN (59 peptides total).

FIG. 11A and FIG. 11B illustrate vaccine-induced HBV-specific CD8+ T cell response in the liver of C57B1/6 mice injected with the indicated vectors. * p-value between 0.01-0.05; *** p-value between 0.0001-0.001; via 1-way ANOVA.

FIG. 12A, FIG. 12B, FIG. 12C, FIG. 12D, FIG. 12E, and FIG. 12F illustrate hematoxylin & eosin staining of liver samples from C57B1/6 mice injected with the indicated vectors. 20× magnification. Arrows indicate areas of lymphocytic infiltrates.

FIG. 13A and FIG. 13B illustrate vaccine-induced markers of CD8+ T cell activation/exhaustion in the liver of C57B1/6 mice injected with the indicated vectors. ** p-value between 0.001-0.01; *** p-value between 0.0001-0.001; via 1-way ANOVA.

FIG. 14A and FIG. 14B illustrate HBV viral dynamics in C57B1/6 mice injected with an exemplary AdC6-gDPolN vector. The median HBV DNA VL/ml at week 4-7.3 log₁₀ cps/mL are provided. n=7; one mouse excluded for missing data.

FIG. 15A and FIG. 15B illustrate the impact of AAV-induced HBV on CD8+ T cell responses in C57B1/6 mice first injected with 10¹⁰ or 10¹¹ vg of AAV-1.3HBV and then four weeks later boosted with 10¹⁰ vp of an exemplary AdC6-gDPolN vector. In FIG. 15B, each slice represents an individual epitope with size showing the proportion of the total; only responses >0.1% were included. Pullouts represent epitopes only recognized in AAV8-1.3HBV infected mice.

FIG. 16 illustrates the frequencies of IFN-γ-producing CD8+ T cells for individual C57B1/6 mice that were injected i.v. with the 10¹⁰ vg of the AAV8-1.3HBV vectors, vaccinated 4 weeks later with 5×10 9 vp of the AdC6-gDPolN vector, and boosted 2 months later with the same dose of the AdC7-gDPolN vaccine. Control mice only received the vaccine. Naïve mice served as additional controls.

FIG. 17A and FIG. 17B illustrate: A) % of CD8⁺ T cells within the lymphatic infiltrates of livers of individual mice; and B) the frequencies of PolN-tetramer⁺CD8⁺ T cells within the same infiltrates. C57B1/6 mice were injected i.v. with the 10¹⁰ or 10¹¹ vg of the AAV8-1.3HBV vector, were vaccinated 4 weeks later with 5×10 9 vp of the AdC6-gDPolN vector, and were boosted 2 months later with the same dose of the AdC7-gDPolN vaccine. Control mice only received the vaccine. Naïve mice served as additional controls.

FIG. 18A, FIG. 18B, FIG. 18C, FIG. 18D, FIG. 18E, and FIG. 18F illustrate the phenotypes of the infiltrating tetramer+CD8⁺ T cells in comparison to naïve (i.e., tetramer CD44⁻CD8⁺) T cells analyzed with the mean fluorescent intensity (MFI) of the indicated markers. Lines with stars above indicate significant differences by multiple t-test. (*) p≤0.05-0.01, (**) p≤0.01-0.001, (***) p≤0.001-0.0001, (****) p≤0.0001.

FIG. 19A, FIG. 19B, FIG. 19C, FIG. 19D, FIG. 19E, and FIG. 19F illustrate the percentage of Tet⁺ or naïve CD8⁺ T cells positive for the indicated markers. Lines with stars above indicate significant differences by multiple t-test. (*) p≤0.05-0.01, (**) p≤0.01-0.001, (***) p≤0.001-0.0001, (****) p≤0.0001.

FIG. 20A, FIG. 20B, FIG. 20C, FIG. 20D, FIG. 20E, and FIG. 20F illustrate the CD8⁺ T cell response to individual peptides spanning the PolN sequence. Total pool—response to mixtures of all PolN peptides; Naïve—response of naïve mice to mixtures of all PolN peptides. FIG. 20A and FIG. 20D show CD8⁺ T cell responses of mice that received just the AdC6-gDPolN vaccine. FIG. 20B and FIG. 20E show CD8⁺ T cell responses of mice that were injected with 10¹⁰ vg of AAV8-1.3HBV 4 weeks prior to vaccination with AdC6-gDPolN. FIG. 20C and FIG. 20F show CD8⁺ T cell responses of mice that were injected with 10¹¹ vg of AAV8-1.3HBV 4 weeks prior to vaccination with AdC6-gDPolN. FIG. 20A, 20B and 20C can be used to calculate the breadth of the immune response by individual epitopes using the peptide pools shown in FIG. 7D and the individual peptide sequences recognized using FIG. 7E.

FIG. 21A and FIG. 21B illustrate the PolN-specific CD8+ T cells in the spleen or liver of mice. FIG. 21A left panel shows CD8⁺ T cell responses in spleen of AAV8-1.3HBV injected mice that did or did not receive the AdC6-gDPolN vaccine at 5×10¹⁰ vp subsequently. FIG. 21A middle panel shows CD8⁺ T cell frequencies in livers of mice that were treated with different doses of AAV8-1.3HBV and then received vaccines in a prime boost regimen. FIG. 21A right panel shows the levels of Tox-1 expression in PolN-specific CD8⁺ T cells or naïve CD8⁺ T cells from the same experiment. FIG. 21B illustrates % IFN-γ⁺CD8⁺ T cells.

FIG. 22A and FIG. 22B illustrate A) CD8+ T cell frequencies in the blood of mice injected with the indicated AdC6 vectors; and B) frequencies of tetramer+CD8+ T cells.

FIG. 23 illustrates CD8+ T cell frequencies in the blood of mice injected with the indicated AdC7 vectors.

FIG. 24A, FIG. 24B, FIG. 24C, FIG. 24D, FIG. 24E, and FIG. 24F illustrate CD8⁺ (FIG. 24A-FIG. 24C) and CD4+ (FIG. 24D-FIG. 24F) T cell frequencies to the gDHBV2 and gDHBV3 inserts in blood of mice injected with the indicated AdC7 vectors (“after prime”) and then boosted with the corresponding AdC6 vectors (“after boost”). Graphs show frequencies of T cells producing IFN-γ, frequencies of T cells producing TNF-α, and the sum of frequencies of T cells producing either cytokine.

FIG. 25A and FIG. 25B illustrate the HBV DNA viral titer in C57B1/6 mice that were challenged with 1×10 9 vg of AAV8-1.3HBV and were vaccinated 4 weeks later with 1×10¹⁰ vp of AdC6-gDPolN (“gDPolN”), AdC6-gDHBV2 (“gDHBV2”), AdC6-gDHBV3 (“gDHBV3”), or AdC6-HBV2 without gD (“HBV2”); AAV-infected, non-vaccinated animals (“naïve”), and non-AAV-infected, non-vaccinated animals (data not shown) served as controls. FIG. 25A illustrates the viral titer for each group at weeks 4 and 8 after AAV challenge; FIG. 25B illustrates the results of the individual mice at weeks 4 and 8 after AAV challenge.

FIG. 26A, FIG. 26B, FIG. 26C, and FIG. 26D illustrate the percent of parental IFN-γ and/or TNF-α producing CD8⁺ T cells (FIG. 26A), CD44+ CD8+ T cells (FIG. 26B), CD4+ T cells (FIG. 26C) or CD44+CD4+ T cells (FIG. 26D) two and eight weeks after prime and two and four weeks after the boost (as the mean) using the indicated construct.

FIG. 27A, FIG. 27B, and FIG. 27C illustrate CD8⁺ T cells at multiple time points: four weeks after prime (FIG. 27A); two weeks after the boost (FIG. 27B); and four weeks after the boost (FIG. 27C) with the indicated constructs (PolN=gDPolN; HBV2=gDHBV2; HBV3=gDHBV3). The graph shows the overall frequencies of CD8⁺ T cells producing IFN-γ⁺ as assessed by ICS.

FIG. 28A, FIG. 28B, and FIG. 28C illustrate cytokine-producing CD4+ T cells at multiple time points: four weeks after prime (FIG. 28A); two weeks after the boost (FIG. 28B); and four weeks after the boost (FIG. 28C) with the indicated constructs (PolN=gDPolN; HBV2=gDHBV2; HBV3=gDHBV3) as assessed by ICS. The dashed line indicates the cut-off for positive responses, based on the results from the naïve mice.

FIG. 29A and FIG. 29B illustrate the results of tetramer staining gated on either CD8+ T cells (FIG. 29A) or CD44+CD8+ T cells (FIG. 29B) at four weeks after the prime with the indicated construct (PolN=gDPolN; HBV2=gDHBV2).

FIG. 30A, FIG. 30B, FIG. 30C, FIG. 30D, FIG. 30E, and FIG. 30F illustrates the phenotypes of the tetramer+CD8+ T cells shown as the mean fluorescent intensity of a dye linked to the indicated antibody: FIG. 30A—anti-PD1 antibody conjugated to BV605; FIG. 30B—anti-LAG3 antibody conjugated to BV650; FIG. 30C—anti-TIM3 antibody conjugated to Pe-Cy7-A; FIG. 30D—anti-CTLA4 antibody conjugated to PE-A; FIG. 30E—anti-EOMES antibody conjugated to AF488; and FIG. 30F—anti-T-bet antibody conjugated to BV786.

FIG. 31 illustrates the CD8⁺ T cell responses after a prime vaccination of 5×10¹⁰ vp AdC7-gDHBV2 followed two months later by vaccination with 5×10¹⁰ vp AdC6-gDHBV2. Numbers on the X axis correspond to the SEQ ID NO as provided herein.

FIG. 32 illustrates the CD8+ T cell responses after a prime vaccination with 5×10⁹ vp AdC7-gDHBV2 followed two months later by vaccination with 5×10⁹ vp AdC6-gDHBV2. Numbers on the X axis correspond to the SEQ ID NO as provided herein.

FIG. 33 shows the immunogenicity after a prime vaccination with 5×10¹⁰ vp AdC7-gDHBV3 followed two months later by vaccination with 5×10¹⁰ vp AdC6-gDHBV3. Numbers on the X axis correspond to the SEQ ID NO as provided herein.

FIG. 34 illustrates the immunogenicity of the AdC6-gDHBV2 and AdC7-gDHBV2 vaccines corresponding to the SEQ ID NO (X axis) as provided herein. Core, PolC, and PolN regions in both HBV2 constructs were immunogenic.

FIG. 35 illustrates the immunogenicity of the AdC6-gDHBV3 and AdC7-gDHBV3 vaccines corresponding to the SEQ ID NO (X axis) as provided herein. Core, PolC, and PolN regions in both HBV3 constructs were immunogenic.

### DETAILED DESCRIPTION OF ILLUSTRATIVE EMBODIMENTS

The disclosed proteins, vaccines, and methods may be understood more readily by reference to the following detailed description taken in connection with the accompanying figures, which form a part of this disclosure. It is to be understood that the disclosed proteins, vaccines, and methods are not limited to the specific proteins, vaccines, and methods described and/or shown herein, and that the terminology used herein is for the purpose of describing particular embodiments by way of example only and is not intended to be limiting of the claimed proteins, vaccines, and methods.

Unless specifically stated otherwise, any description as to a possible mechanism or mode of action or reason for improvement is meant to be illustrative only, and the disclosed proteins, vaccines, and methods are not to be constrained by the correctness or incorrectness of any such suggested mechanism or mode of action or reason for improvement.

Throughout this text, the descriptions refer to proteins and methods of using said proteins. Where the disclosure describes or claims a feature or embodiment associated with a proteins, such a feature or embodiment is equally applicable to the methods of using said proteins. Likewise, where the disclosure describes or claims a feature or embodiment associated with a method of using the proteins, such a feature or embodiment is equally applicable to the proteins.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed proteins, vaccines, and methods which are, for clarity, described herein in the context of separate embodiments, may also be provided in combination in a single embodiment. Conversely, various features of the disclosed proteins, vaccines, and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “immunogenic fragment thereof” refers to a portion of the disclosed HBV Core (Core), HBV polymerase N-terminal domain (PolN), or HBV polymerase C-terminal domain (PolC) that can produce an immune response in a subject.

As used herein, “providing to the subject” and similar terms indicate a procedure by which the fusion proteins, nucleic acid molecules, vectors, or vaccines are delivered to a subject such that target cells, tissues, or segments of the body of the subject are contacted with the fusion proteins, nucleic acid molecules, vectors, or vaccines. “Providing to the subject” includes parenteral and non-parenteral routes of administration.

The term “biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extent the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance. Biosimilars of the disclosed proteins and fusion proteins are included within the scope of this disclosure.

The term “subject” as used herein is intended to mean any animal, in particular, mammals. Although induction of an immune response in mice is exemplified herein, any type of mammal can be treated using the disclosed methods. Thus, the methods are applicable to human and nonhuman animals, although preferably used with mice and humans, and most preferably with humans.

The term “comprising” is intended to include examples encompassed by the terms “consisting essentially of” and “consisting of”; similarly, the term “consisting essentially of” is intended to include examples encompassed by the term “consisting of”

The following abbreviations are used herein: hepatitis B virus (HBV); adenovirus (Ad); herpes simplex virus (HSV); glycoprotein (gD); and virus genomes (vg).

Provided herein is a non-naturally occurring variant of the hepatitis B virus (HBV) Core protein. The disclosed HBV Core protein can comprise the amino acid sequence of SEQ ID NO: 6 or an immunogenic fragment thereof. Exemplary immunogenic fragments of SEQ ID NO: 6 include SEQ ID NOs: 20-54 provided in Table 3, below. In some embodiments, the immunogenic fragment of the HBV Core protein comprises the amino acid sequence of SEQ ID NO: 180. In some embodiments, the immunogenic fragment of the HBV Core protein comprises the amino acid sequence of SEQ ID NO: 183.

Nucleic acid molecules encoding the HBV Core protein or an immunogenic fragment thereof are also provided. The nucleic acid molecule can encode the HBV Core protein comprising the amino acid sequence of SEQ ID NO: 6. In some embodiments, the nucleic acid molecule comprises the nucleotide sequence of SEQ ID NO: 7. The nucleic acid molecules can encode the Core fragments provided in Table 3. In some embodiments, the nucleic acid molecule encodes the amino acid sequence of SEQ ID NO: 180. In some embodiments, the nucleic acid molecule encodes the amino acid sequence of SEQ ID NO: 183.

Vectors comprising the nucleic acid molecules encoding the HBV Core protein or an immunogenic fragment thereof are also provided. Suitable vectors include viral vectors, such as lentiviral vectors, retroviral vectors, adenoviral vectors, adeno-associated viral vectors, alphavirus replicons, herpes virus vectors, pox virus vectors, and rhabdovirus vectors. In some embodiments, the viral vector is an adenoviral vector. The adenoviral vector can be a chimpanzee-derived adenoviral vector. In some aspects, the vector is an AdC68 vector as described in Farina S F, Gao G P, Xiang Z Q, Rux J J, Burnett R M, Alvira M R, Marsh J, Ertl H C, Wilson J M. “Replication-defective vector based on a chimpanzee adenovirus.” J Virol. 2001 Dec; 75(23): 11603-13. In some aspects, the vector is an AdC7 vector as described in Reyes-Sandoval A, Fitzgerald J C, Grant R, Roy S, Xiang Z Q, Li Y, Gao G P, Wilson J M, Ertl H C. “Human immunodeficiency virus type 1-specific immune responses in primates upon sequential immunization with adenoviral vaccine carriers of human and simian serotypes”J Virol. 2004 Jul; 78(14): 7392-9. In some aspects, the vector is an AdC6 vector as described in Pinto A R, Fitzgerald J C, Giles-Davis W, Gao G P, Wilson J M, Ertl H C. “Induction of CD8+ T cells to an HIV-1 antigen through a prime boost regimen with heterologous El-deleted adenoviral vaccine carriers” J Immunol. 2003 Dec. 15; 171(12): 6774-9.

In some embodiments, the vector comprises the nucleic acid molecule comprising the nucleotide sequence of SEQ ID NO: 7. In some embodiments, the vector is an AdC6 vector comprising the nucleic acid molecule comprising the nucleotide sequence of SEQ ID NO: 7. In some embodiments, the vector is an AdC7 vector comprising the nucleic acid molecule comprising the nucleotide sequence of SEQ ID NO: 7.

In some embodiments, the vector comprises the nucleic acid molecule that encodes the amino acid sequence of SEQ ID NO: 180. In some aspects, the vector is an AdC6 vector. In some aspects, the vector is an AdC7 vector. In some embodiments, the vector comprises the nucleic acid molecule that encodes the amino acid sequence of SEQ ID NO: 183. In some aspects, the vector is an AdC6 vector. In some aspects, the vector is an AdC7 vector.

Vaccines comprising the vectors comprising the nucleic acid molecules encoding the HBV Core protein or an immunogenic fragment thereof are also disclosed. In some embodiments, the vaccine comprises a vector comprising the nucleic acid molecule comprising the nucleotide sequence of SEQ ID NO: 7. In some embodiments, the vaccine comprises an AdC6 vector comprising the nucleic acid molecule comprising the nucleotide sequence of SEQ ID NO: 7. In some embodiments, the vaccine comprises an AdC7 vector comprising the nucleic acid molecule comprising the nucleotide sequence of SEQ ID NO: 7. In some embodiments, the vaccine comprises an AdC6 vector comprising the nucleic acid molecule that encodes the amino acid sequence of SEQ ID NO: 180. In some embodiments, the vaccine comprises an AdC7 vector comprising the nucleic acid molecule that encodes the amino acid sequence of SEQ ID NO: 180. In some embodiments, the vaccine comprises an AdC6 vector that comprises the nucleic acid molecule that encodes the amino acid sequence of SEQ ID NO: 183. In some embodiments, the vaccine comprises an AdC7 vector that comprises the nucleic acid molecule that encodes the amino acid sequence of SEQ ID NO: 183.

The vaccine can further comprise a pharmaceutically acceptable carrier or pharmaceutical acceptable excipient. As used herein, “pharmaceutically acceptable carrier” or “pharmaceutical acceptable excipient” includes any material which, when combined with the disclosed fusion proteins, nucleic acids, or vectors, allows the fusion proteins, nucleic acids, or vectors to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

Also disclosed herein are non-naturally occurring variants of the HBV polymerase N-terminal domain (PolN) and the HBV polymerase C-terminal domain (PolC). The disclosed HBV polymerase N-terminal domain can comprise the amino acid sequence of SEQ ID NO: 8 or an immunogenic fragment thereof Exemplary immunogenic fragments of SEQ ID NO: 8 include SEQ ID NOs: 55-113 provided in Table 4, below. In some embodiments, the immunogenic fragment of the HBV PolN comprises the amino acid sequence of SEQ ID NO: 178. In some embodiments, the immunogenic fragment of the HBV PolN comprises the amino acid sequence of SEQ ID NO: 181. The disclosed HBV polymerase C-terminal domain can comprise the amino acid sequence of SEQ ID NO: 10 or an immunogenic fragment thereof. Exemplary immunogenic fragments of SEQ ID NO: 10 include SEQ ID NOs: 114-172 provided in Table 5, below. In some embodiments, the immunogenic fragment of the HBV PolC comprises the amino acid sequence of SEQ ID NO: 179. In some embodiments, the immunogenic fragment of the HBV PolC comprises the amino acid sequence of SEQ ID NO: 182.

Nucleic acid molecules encoding the HBV polymerase N-terminal domain or an immunogenic fragment thereof, or the HBV polymerase C-terminal domain or an immunogenic fragment thereof, are also provided. The nucleic acid molecule can encode the HBV polymerase N-terminal domain comprising the amino acid sequence of SEQ ID NO: 8. In some embodiments, the nucleic acid molecule encoding the HBV polymerase N-terminal domain comprises the nucleotide sequence of SEQ ID NO: 9. The nucleic acid molecules can encode the HBV polymerase N-terminal domain fragments provided in Table 4. The nucleic acid molecule can encode the HBV polymerase C-terminal domain comprising the amino acid sequence of SEQ ID NO: 10. In some embodiments, the nucleic acid molecule encoding the HBV polymerase C-terminal domain comprises the nucleotide sequence of SEQ ID NO: 11. The nucleic acid molecules can encode the HBV polymerase C-terminal domain fragments provided in Table 5. In some embodiments, the nucleic acid molecule encodes the amino acid sequence of SEQ ID NO: 178. In some embodiments, the nucleic acid molecule encodes the amino acid sequence of SEQ ID NO: 181. In some embodiments, the nucleic acid molecule encodes the amino acid sequence of SEQ ID NO: 179. In some embodiments, the nucleic acid molecule encodes the amino acid sequence of SEQ ID NO: 182.

Vectors comprising the nucleic acid molecules encoding the HBV polymerase N-terminal domain or an immunogenic fragment thereof or C-terminal domain or an immunogenic fragment thereof are also provided. Suitable vectors include those described above. In some embodiments, the vector comprises the nucleic acid molecule comprising the nucleotide sequence of SEQ ID NO: 9. In some embodiments, the vector comprises the nucleic acid molecule comprising the nucleotide sequence of SEQ ID NO: 11. In some aspects, the vector is an adenoviral vector. Suitable adenoviral vectors include, for example, an AdC6 vector or AdC7 vector. In some embodiments, the vector is an AdC6 vector comprising the nucleic acid molecule comprising the nucleotide sequence of SEQ ID NO: 9. In some embodiments, the vector is an AdC7 vector comprising the nucleic acid molecule comprising the nucleotide sequence of SEQ ID NO: 9. In some embodiments, the vector is an AdC6 vector comprising the nucleic acid molecule comprising the nucleotide sequence of SEQ ID NO: 11. In some embodiments, the vector is an AdC7 vector comprising the nucleic acid molecule comprising the nucleotide sequence of SEQ ID NO: 11. In some embodiments, the vector comprises the nucleic acid molecule that encodes the amino acid sequence of SEQ ID NO: 178. In some aspects, the vector is an AdC6 vector. In some aspects, the vector is an AdC7 vector. In some embodiments, the vector comprises the nucleic acid molecule that encodes the amino acid sequence of SEQ ID NO: 181. In some aspects, the vector is an AdC6 vector. In some aspects, the vector is an AdC7 vector. In some embodiments, the vector comprises the nucleic acid molecule that encodes the amino acid sequence of SEQ ID NO: 179. In some aspects, the vector is an AdC6 vector. In some aspects, the vector is an AdC7 vector. In some embodiments, the vector comprises the nucleic acid molecule that encodes the amino acid sequence of SEQ ID NO: 182. In some aspects, the vector is an AdC6 vector. In some aspects, the vector is an AdC7 vector.

Vaccines comprising the vectors comprising the nucleic acid molecules encoding the HBV polymerase N-terminal domain or an immunogenic fragment thereof or HBV polymerase C-terminal domain or an immunogenic fragment thereof are also disclosed. In some embodiments, the vaccine comprises a vector comprising the nucleic acid molecule comprising the nucleotide sequence of SEQ ID NO: 9. The vaccine can comprise an AdC6 vector comprising the nucleic acid molecule comprising the nucleotide sequence of SEQ ID NO: 9. The vaccine can comprise an AdC7 vector comprising the nucleic acid molecule comprising the nucleotide sequence of SEQ ID NO: 9. In some embodiments, the vaccine comprises a vector comprising the nucleic acid molecule comprising the nucleotide sequence of SEQ ID NO: 11. The vaccine can comprise an AdC6 vector comprising the nucleic acid molecule comprising the nucleotide sequence of SEQ ID NO: 11. The vaccine can comprise an AdC7 vector comprising the nucleic acid molecule comprising the nucleotide sequence of SEQ ID NO: 11. The vaccine can further comprise a pharmaceutically acceptable carrier or pharmaceutical acceptable excipient as disclosed above. In some embodiments, the vaccine comprises a vector comprising the nucleic acid molecule that encodes the amino acid sequence of SEQ ID NO: 178. In some aspects, the vector is an AdC6 vector. In some aspects, the vector is an AdC7 vector. In some embodiments, the vaccine comprises a vector comprising the nucleic acid molecule that encodes the amino acid sequence of SEQ ID NO: 181. In some aspects, the vector is an AdC6 vector. In some aspects, the vector is an AdC7 vector. In some embodiments, the vaccine comprises a vector comprising the nucleic acid molecule that encodes the amino acid sequence of SEQ ID NO: 179. In some aspects, the vector is an AdC6 vector. In some aspects, the vector is an AdC7 vector. In some embodiments, the vaccine comprises a vector comprising the nucleic acid molecule that encodes the amino acid sequence of SEQ ID NO: 182. In some aspects, the vector is an AdC6 vector. In some aspects, the vector is an AdC7 vector.

Fusion proteins comprising combinations of the disclosed HBV Core protein or immunogenic fragments thereof, the HBV polymerase N-terminal domain or immunogenic fragments thereof, and/or the HBV polymerase C-terminal domain or immunogenic fragments thereof are also provided herein. For example, the fusion protein can comprise:
(1) an HBV Core protein comprising the amino acid sequence of SEQ ID NO: 6 or an immunogenic fragment thereof and an HBV polymerase N-terminal domain comprising the amino acid sequence of SEQ ID NO: 8 or an immunogenic fragment thereof;
(2) one or more immunogenic fragments of the HBV Core protein comprising the amino acid sequence of SEQ ID NO: 6 and one or more immunogenic fragments of the HBV polymerase N-terminal domain comprising the amino acid sequence of SEQ ID NO: 8. For example, one or more of SEQ ID NOs: 20-54 provided in Table 3 (immunogenic fragments of SEQ ID NO: 6) and one or more of SEQ ID NOs: 55-113 provided in Table 4 (immunogenic fragments of SEQ ID NO: 8);
(3) an HBV Core protein comprising the amino acid sequence of SEQ ID NO: 6 or an immunogenic fragment thereof and an HBV polymerase C-terminal domain comprising the amino acid sequence of SEQ ID NO: 10 or an immunogenic fragment thereof;
(4) one or more immunogenic fragments of the HBV Core protein comprising the amino acid sequence of SEQ ID NO: 6 and one or more immunogenic fragments of the HBV polymerase C-terminal domain comprising the amino acid sequence of SEQ ID NO: 10. For example, one or more of SEQ ID NOs: 20-54 provided in Table 3 (immunogenic fragments of SEQ ID NO: 6) and one or more of SEQ ID NOs: 114-172 provided in Table 5 (immunogenic fragments of SEQ ID NO: 10);
(5) an HBV polymerase N-terminal domain comprising the amino acid sequence of

SEQ ID NO: 8 or an immunogenic fragment thereof and an HBV polymerase C-terminal domain comprising the amino acid sequence of SEQ ID NO: 10 or an immunogenic fragment thereof;
(6) one or more immunogenic fragments of the HBV polymerase N-terminal domain comprising the amino acid sequence of SEQ ID NO: 8 and one or more immunogenic fragments of the HBV polymerase C-terminal domain comprising the amino acid sequence of SEQ ID NO: 10. For example, one or more of SEQ ID NOs: 55-113 provided in Table 4 (immunogenic fragments of SEQ ID NO: 8) and one or more of SEQ ID NOs: 114-172 provided in Table 5 (immunogenic fragments of SEQ ID NO: 10);
(7) an HBV Core protein comprising the amino acid sequence of SEQ ID NO: 6 or an immunogenic fragment thereof, an HBV polymerase N-terminal domain comprising the amino acid sequence of SEQ ID NO: 8 or an immunogenic fragment thereof, and an HBV polymerase C-terminal domain comprising the amino acid sequence of SEQ ID NO: 10 or an immunogenic fragment thereof;
(8) one or more immunogenic fragments of the HBV Core protein comprising the amino acid sequence of SEQ ID NO: 6, one or more immunogenic fragments of the HBV polymerase N-terminal domain comprising the amino acid sequence of SEQ ID NO: 8, and one or more immunogenic fragments of the HBV polymerase C-terminal domain comprising the amino acid sequence of SEQ ID NO: 10. For example, one or more of SEQ ID NOs: 20-54 provided in Table 3 (immunogenic fragments of SEQ ID NO: 6), one or more of SEQ ID NOs: 55-113 provided in Table 4 (immunogenic fragments of SEQ ID NO: 8), and one or more of SEQ ID NOs: 114-172 provided in Table 5 (immunogenic fragments of SEQ ID NO: 10);
(9) An HBV polymerase N-terminal domain comprising the amino acid sequence of SEQ ID NO: 178 or an immunogenic fragment thereof, an HBV polymerase C-terminal domain comprising the amino acid sequence of SEQ ID NO: 179 or an immunogenic fragment thereof, and an HBV Core protein comprising the amino acid sequence of SEQ ID NO: 180 or an immunogenic fragment thereof; or
(10) An HBV polymerase N-terminal domain comprising the amino acid sequence of SEQ ID NO: 181 or an immunogenic fragment thereof, an HBV polymerase C-terminal domain comprising the amino acid sequence of SEQ ID NO: 182 or an immunogenic fragment thereof, and an HBV Core protein comprising the amino acid sequence of SEQ ID NO: 183 or an immunogenic fragment thereof.

The fusion protein can comprise an HBV polymerase N-terminal domain comprising the amino acid sequence of SEQ ID NO: 178 or an immunogenic fragment thereof, an HBV polymerase C-terminal domain comprising the amino acid sequence of SEQ ID NO: 179 or an immunogenic fragment thereof, and an HBV Core protein comprising the amino acid sequence of SEQ ID NO: 180 or an immunogenic fragment thereof. In some embodiments, the fusion protein comprises the amino acid sequence of SEQ ID NO: 174.

The fusion protein can comprise an HBV polymerase N-terminal domain comprising the amino acid sequence of SEQ ID NO: 181 or an immunogenic fragment thereof, an HBV polymerase C-terminal domain comprising the amino acid sequence of SEQ ID NO: 182 or an immunogenic fragment thereof, and an HBV Core protein comprising the amino acid sequence of SEQ ID NO: 183 or an immunogenic fragment thereof. In some embodiments, the fusion protein comprises the amino acid sequence of SEQ ID NO: 175.

Also provided herein are fusion proteins comprising a herpes simplex virus (HSV) glycoprotein (gD) sequence and the disclosed HBV Core protein, the HBV polymerase N-terminal domain, the HBV polymerase C-terminal domain, or various combinations thereof

The HSV gD is a receptor-binding glycoprotein of HSV. The gD ectodomain is organized in two structurally and functionally differentiated regions: the amino-terminus, which includes the signal sequence and receptor-binding sites; and the carboxy-terminus, which includes the pro-fusion domain and the transmembrane domain. gD interacts with the herpesvirus entry mediator (HVEM) receptor and the nectin receptors. Interaction of gD with the receptors results in the down-regulation of the HVEM receptors binding to BTLA or CD160, which are immunoinhibitory molecules that are expressed on T cells. In some embodiments, the disclosed fusion proteins comprising gD and the disclosed HBV Core protein, the HBV polymerase N-terminal domain, the HBV polymerase C-terminal domain (referred to as “gDCore,” “gDPolN” or “gDPolC,” respectively), or combinations thereof are expected to enhance a subject's immune response against HBV to a greater extent compared to the HBV Core and/or polymerase antigens alone (i.e. without gD).

Suitable HSV gD proteins for use in the disclosed fusion proteins include wild-type or mutant gD that retains the ability to: 1) augment stimulation of a CD8+ T cell response to an antigen; and/or 2) disrupt an HVEM-BTLA pathway activity.

The fusion proteins can comprise the HBV Core protein or an immunogenic fragment thereof, HBV polymerase N-terminal domain or an immunogenic fragment thereof, HBV polymerase C-terminal domain or an immunogenic fragment thereof disclosed herein, or any combination thereof, an N-terminal HSV gD protein sequence, and a C-terminal HSV gD protein sequence. The HBV Core protein, HBV polymerase N-terminal domain, and HBV polymerase C-terminal domain can be those provided in Table 9 or the immunogenic fragments provided in Tables 3-5. The HBV Core protein, HBV polymerase N-terminal domain, HBV polymerase C-terminal domain, or immunogenic fragments thereof can be inserted between the N-terminal HSV gD protein sequence and the C-terminal HSV gD protein sequence. In some aspects, the N-terminal HSV gD protein sequence comprises the amino acid sequence of SEQ ID NO: 12 and the C-terminal HSV gD protein sequence comprises the amino acid sequence of SEQ ID NO: 13. In some embodiments, the N-terminal HSV gD protein sequence comprises amino acid residues 26-269 of SEQ ID NO: 12.

The fusion protein can comprise:
an N-terminal HSV gD sequence or a variant thereof;
an HBV Core protein comprising the amino acid sequence of SEQ ID NO: 6 or an immunogenic fragment thereof; and
a C-terminal HSV gD sequence or a variant thereof.

The immunogenic fragment of the HBV Core protein can comprise any one of SEQ ID NOs: 20-54, 180, or 183.

The fusion protein can comprise:
an N-terminal HSV gD sequence or a variant thereof;
an HBV Core protein comprising the amino acid sequence of SEQ ID NO: 180 or SEQ ID NO: 183; and
a C-terminal HSV gD sequence or a variant thereof.

The fusion protein can comprise:
an N-terminal HSV gD sequence or a variant thereof;
an HBV polymerase N-terminal domain comprising the amino acid sequence of SEQ ID NO: 8 or an immunogenic fragment thereof; and
a C-terminal HSV gD protein sequence or a variant thereof.

The immunogenic fragment of the HBV polymerase N-terminal domain can comprise any one of SEQ ID NOs: 55-113, 178, or 181.

The fusion protein can comprise:
an N-terminal HSV gD sequence or a variant thereof;
an HBV polymerase N-terminal domain comprising the amino acid sequence of SEQ ID NO: 178 or SEQ ID NO: 181; and
a C-terminal HSV gD protein sequence or a variant thereof.

The fusion protein can comprise:
an N-terminal HSV gD sequence or a variant thereof;
an HBV polymerase C-terminal domain comprising the amino acid sequence of SEQ ID NO: 10 or an immunogenic fragment thereof; and
a C-terminal HSV gD protein sequence or a variant thereof.

The immunogenic fragment of the HBV polymerase C-terminal domain can comprise any one of SEQ ID NOs: 114-172, 179, or 182.

The fusion protein can comprise:
an N-terminal HSV gD sequence or a variant thereof;
an HBV polymerase C-terminal domain comprising the amino acid sequence of SEQ ID NO: 179 or SEQ ID NO: 182; and
a C-terminal HSV gD protein sequence or a variant thereof.

The fusion protein can comprise:
an N-terminal HSV gD sequence or a variant thereof;
an HBV sequence comprising:
   (1) an HBV Core protein comprising the amino acid sequence of SEQ ID NO: 6 or an immunogenic fragment thereof and an HBV polymerase N-terminal domain comprising the amino acid sequence of SEQ ID NO: 8 or an immunogenic fragment thereof;
   (2) one or more immunogenic fragments of the HBV Core protein comprising the amino acid sequence of SEQ ID NO: 6 and one or more immunogenic fragments of the HBV polymerase N-terminal domain comprising the amino acid sequence of SEQ ID NO: 8. For example, one or more of SEQ ID NOs: 20-54 provided in Table 3 (immunogenic fragments of SEQ ID NO: 6) and one or more of SEQ ID NOs: 55-113 provided in Table 4 (immunogenic fragments of SEQ ID NO: 8);
   (3) an HBV Core protein comprising the amino acid sequence of SEQ ID NO: 6 or an immunogenic fragment thereof and an HBV polymerase C-terminal domain comprising the amino acid sequence of SEQ ID NO: 10 or an immunogenic fragment thereof;
   (4) one or more immunogenic fragments of the HBV Core protein comprising the amino acid sequence of SEQ ID NO: 6 and one or more immunogenic fragments of the HBV polymerase C-terminal domain comprising the amino acid sequence of SEQ ID NO: 10. For example, one or more of SEQ ID NOs: 20-54 provided in Table 3 (immunogenic fragments of SEQ ID NO: 6) and one or more of SEQ ID NOs: 114-172 provided in Table 5 (immunogenic fragments of SEQ ID NO: 10);
   (5) an HBV polymerase N-terminal domain comprising the amino acid sequence of SEQ ID NO: 8 or an immunogenic fragment thereof and an HBV polymerase C-terminal domain comprising the amino acid sequence of SEQ ID NO: 10 or an immunogenic fragment thereof;
   (6) one or more immunogenic fragments of the HBV polymerase N-terminal domain comprising the amino acid sequence of SEQ ID NO: 8 and one or more immunogenic fragments of the HBV polymerase C-terminal domain comprising the amino acid sequence of SEQ ID NO: 10. For example, one or more of SEQ ID NOs: 55-113 provided in Table 4 (immunogenic fragments of SEQ ID NO: 8) and one or more of SEQ ID NOs: 114-172 provided in Table 5 (immunogenic fragments of SEQ ID NO: 10);
   (7) an HBV Core protein comprising the amino acid sequence of SEQ ID NO: 6 or an immunogenic fragment thereof, an HBV polymerase N-terminal domain comprising the amino acid sequence of SEQ ID NO: 8 or an immunogenic fragment thereof, and an HBV polymerase C-terminal domain comprising the amino acid sequence of SEQ ID NO: 10 or an immunogenic fragment thereof; or
   (8) one or more immunogenic fragments of the HBV Core protein comprising the amino acid sequence of SEQ ID NO: 6, one or more immunogenic fragments of the HBV polymerase N-terminal domain comprising the amino acid sequence of SEQ ID NO: 8, and one or more immunogenic fragments of the HBV polymerase C-terminal domain comprising the amino acid sequence of SEQ ID NO: 10. For example, one or more of SEQ ID NOs: 20-54 provided in Table 3 (immunogenic fragments of SEQ ID NO: 6), one or more of SEQ ID NOs: 55-113 provided in Table 4 (immunogenic fragments of SEQ ID NO: 8), and one or more of SEQ ID NOs: 114-172 provided in Table 5 (immunogenic fragments of SEQ ID NO: 10);
   (9) an HBV polymerase N-terminal domain comprising the amino acid sequence of SEQ ID NO: 178 or an immunogenic fragment thereof, an HBV polymerase C-terminal domain comprising the amino acid sequence of SEQ ID NO: 179 or an immunogenic fragment thereof, and an HBV Core protein comprising the amino acid sequence of SEQ ID NO: 180 or an immunogenic fragment thereof; or
   (10) an HBV polymerase N-terminal domain comprising the amino acid sequence of SEQ ID NO: 181 or an immunogenic fragment thereof, an HBV polymerase C-terminal domain comprising the amino acid sequence of SEQ ID NO: 182 or an immunogenic fragment thereof, and an HBV Core protein comprising the amino acid sequence of SEQ ID NO: 183 or an immunogenic fragment thereof; and
a C-terminal HSV gD protein sequence or a variant thereof. In some embodiments, the N-terminal HSV gD sequence can comprise at least amino acids 1-269 of HSV gD. The N-terminal HSV gD sequence, for example, can comprise the amino acid sequence of SEQ ID NO: 12. In some embodiments, the N-terminal HSV gD sequence comprises amino acid residues 26-269 of SEQ ID NO: 12.

In some embodiments, the C-terminal HSV gD sequence comprises the transmembrane domain of the HSV gD. The C-terminal HSV gD sequence, for example, can comprise the amino acid sequence of SEQ ID NO: 13.

The fusion protein can comprise the amino acid sequence of SEQ ID NO: 14 (corresponding to gDCore) or an immunogenic fragment thereof. The fusion protein can comprise the amino acid sequence of SEQ ID NO: 16 (corresponding to gDPolN) or an immunogenic fragment thereof. The fusion protein can comprise the amino acid sequence of SEQ ID NO: 18 (corresponding to gDPolC) or an immunogenic fragment thereof. In some embodiments, the amino acid sequence of any one of SEQ ID NOs: 14, 16, or 18, or the immunogenic fragment thereof, does not contain the N-terminal 25 amino acid signal peptide.

The fusion protein can comprise the amino acid sequence of SEQ ID NO: 185 (gDHBV2). The fusion protein can comprise the amino acid sequence of SEQ ID NO: 187 (gDHBV3).

Nucleic acid molecules encoding any of the disclosed fusion proteins are also provided. In some embodiments, the nucleic acid molecule comprises the nucleotide sequence of SEQ ID NO: 15 (corresponding to gDCore). In some embodiments, the nucleic acid molecule comprises the nucleotide sequence of SEQ ID NO: 17 (corresponding to gDPolN). In some embodiments, the nucleic acid molecule comprises the nucleotide sequence of SEQ ID NO: 19 (corresponding to gDPolC).

The nucleic acid molecule can comprise the nucleotide sequence of SEQ ID NO: 184 (gDHBV2). The nucleic acid molecule can comprise the nucleotide sequence of SEQ ID NO: 186 (gDHBV3).

Vectors comprising the nucleic acid molecules encoding the fusion proteins are also disclosed. Suitable vectors include those described above including, for example, an adenoviral vector. In some embodiments, the adenoviral vector is an AdC6 vector. In some embodiments, the adenoviral vector is an AdC7 vector. The vector can comprise the nucleotide sequence of SEQ ID NO: 184 (gDHBV2). In some aspects, the vector is an AdC6 vector that comprises the nucleotide sequence of SEQ ID NO: 184 (gDHBV2). In some aspects, the vector is an AdC7 vector that comprises the nucleotide sequence of SEQ ID NO: 184 (gDHBV2). The vector can comprise the nucleotide sequence of SEQ ID NO: 186 (gDHBV3). In some aspects, the vector is an AdC6 vector that comprises the nucleotide sequence of SEQ ID NO: 186 (gDHBV3). In some aspects, the vector is an AdC7 vector that comprises the nucleotide sequence of SEQ ID NO: 186 (gDHBV3).

Vaccines comprising any of the disclosed vectors are also provided. The vaccine can further comprise a pharmaceutically acceptable carrier or pharmaceutical acceptable excipient as disclosed above. The vaccine can comprise a vector comprising the nucleotide sequence of SEQ ID NO: 184 (gDHBV2). In some aspects, the vaccine comprises an AdC6 vector that comprises the nucleotide sequence of SEQ ID NO: 184 (gDHBV2). In some aspects, the vaccine comprises an AdC7 vector that comprises the nucleotide sequence of SEQ ID NO: 184 (gDHBV2). The vaccine can comprise a vector that comprises the nucleotide sequence of SEQ ID NO: 186 (gDHBV3). In some aspects, the vaccine comprises an AdC6 vector that comprises the nucleotide sequence of SEQ ID NO: 186 (gDHBV3). In some aspects, the vaccine comprises an AdC7 vector that comprises the nucleotide sequence of SEQ ID NO: 186 (gDHBV3).

Provided herein are methods of inducing an immune response to HBV in a subject, the methods comprising providing to the subject an effective amount of any of the disclosed fusion proteins, any of the disclosed nucleic acid molecules, any of the disclosed vectors, or any of the disclosed vaccines to thereby induce an immune response to HBV. In some embodiments, the methods comprise providing to the subject an effective amount of any of the disclosed fusion proteins to thereby induce an immune response to HBV. In some embodiments, the methods comprise providing to the subject an effective amount of any of the disclosed nucleic acid molecules to thereby induce an immune response to HBV. In some embodiments, the methods comprise providing to the subject an effective amount of any of the disclosed vectors to thereby induce an immune response to HBV. In some embodiments, the methods comprise providing to the subject an effective amount of any of the disclosed vaccines to thereby induce an immune response to HBV.

The methods can comprise providing to the subject an effective amount of a vaccine comprising an AdC6 vector, wherein the AdC6 vector comprises a fusion protein comprising the amino acid sequence of any one of SEQ ID NOs: 14, 16, or 18, or an immunogenic fragment thereof. In some embodiments, the methods further comprise providing to the subject, subsequent to providing the vaccine comprising the AdC6 vector, a vaccine comprising an AdC7 vector comprising a fusion protein comprising the amino acid sequence of any one of SEQ ID NOs: 14, 16, or 18, or an immunogenic fragment thereof. Such prime-boost methods can comprise:
Providing to the subject a vaccine comprising an AdC6 vector comprising a fusion protein comprising the amino acid sequence of SEQ ID NO: 14, or an immunogenic fragment thereof, and subsequently providing to the subject a vaccine comprising an AdC7 vector comprising a fusion protein comprising the amino acid sequence of SEQ ID NO: 14, or an immunogenic fragment thereof. In some embodiments, the amino acid sequence of SEQ ID NO: 14, or an immunogenic fragment thereof, does not contain the N-terminal 25 amino acid signal peptide;
Providing to the subject a vaccine comprising an AdC6 vector comprising a fusion protein comprising the amino acid sequence of SEQ ID NO: 16, or an immunogenic fragment thereof, and subsequently providing to the subject a vaccine comprising an AdC7 vector comprising a fusion protein comprising the amino acid sequence of SEQ ID NO: 16, or an immunogenic fragment thereof. In some embodiments, the amino acid sequence of SEQ ID NO: 16, or an immunogenic fragment thereof, does not contain the N-terminal 25 amino acid signal peptide; or
Providing to the subject a vaccine comprising an AdC6 vector comprising a fusion protein comprising the amino acid sequence of SEQ ID NO: 18, or an immunogenic fragment thereof, and subsequently providing to the subject a vaccine comprising an AdC7 vector comprising a fusion protein comprising the amino acid sequence of SEQ ID NO: 18, or an immunogenic fragment thereof. In some embodiments, the amino acid sequence of SEQ ID NO: 18, or an immunogenic fragment thereof, does not contain the N-terminal 25 amino acid signal peptide.

The methods can comprise providing to the subject an effective amount of a vaccine comprising an AdC7 vector, wherein the AdC7 vector comprises a fusion protein comprising the amino acid sequence of any one of SEQ ID NOs: 14, 16, or 18, or an immunogenic fragment thereof. In some embodiments, the methods further comprise providing to the subject, subsequent to providing the vaccine comprising the AdC7 vector, a vaccine comprising an AdC6 vector comprising a fusion protein comprising the amino acid sequence of any one of SEQ ID NOs: 14, 16, or 18, or an immunogenic fragment thereof. Such prime-boost methods can comprise:
Providing to the subject a vaccine comprising an AdC7 vector comprising a fusion protein comprising the amino acid sequence of SEQ ID NO: 14, or an immunogenic fragment thereof, and subsequently providing to the subject a vaccine comprising an AdC6 vector comprising a fusion protein comprising the amino acid sequence of SEQ ID NO: 14, or an immunogenic fragment thereof. In some embodiments, the amino acid sequence of SEQ ID NO: 14, or an immunogenic fragment thereof, does not contain the N-terminal 25 amino acid signal peptide;
Providing to the subject a vaccine comprising an AdC7 vector comprising a fusion protein comprising the amino acid sequence of SEQ ID NO: 16, or an immunogenic fragment thereof, and subsequently providing to the subject a vaccine comprising an AdC6 vector comprising a fusion protein comprising the amino acid sequence of SEQ ID NO: 16, or an immunogenic fragment thereof. In some embodiments, the amino acid sequence of SEQ ID NO: 16, or an immunogenic fragment thereof, does not contain the N-terminal 25 amino acid signal peptide; or
Providing to the subject a vaccine comprising an AdC7 vector comprising a fusion protein comprising the amino acid sequence of SEQ ID NO: 18, or an immunogenic fragment thereof, and subsequently providing to the subject a vaccine comprising an AdC6 vector comprising a fusion protein comprising the amino acid sequence of SEQ ID NO: 18, or an immunogenic fragment thereof. In some embodiments, the amino acid sequence of SEQ ID NO: 18, or an immunogenic fragment thereof, does not contain the N-terminal 25 amino acid signal peptide.

The methods can comprise providing to the subject an effective amount of a vaccine comprising an AdC6 vector, wherein the AdC6 vector comprises a fusion protein comprising the amino acid sequence of SEQ ID NO: 185 or 187, or an immunogenic fragment thereof. In some embodiments, the methods further comprise providing to the subject, subsequent to providing the vaccine comprising the AdC6 vector, a vaccine comprising an AdC7 vector comprising a fusion protein comprising the amino acid sequence of SEQ ID NO: 185 or 187, or an immunogenic fragment thereof. Such prime-boost methods can comprise:
Providing to the subject a vaccine comprising an AdC6 vector comprising a fusion protein comprising the amino acid sequence of SEQ ID NO: 185, or an immunogenic fragment thereof, and subsequently providing to the subject a vaccine comprising an AdC7 vector comprising a fusion protein comprising the amino acid sequence of SEQ ID NO: 185, or an immunogenic fragment thereof. In some embodiments, the amino acid sequence of SEQ ID NO: 185, or an immunogenic fragment thereof, does not contain the N-terminal 25 amino acid signal peptide; or
Providing to the subject a vaccine comprising an AdC6 vector comprising a fusion protein comprising the amino acid sequence of SEQ ID NO: 187, or an immunogenic fragment thereof, and subsequently providing to the subject a vaccine comprising an AdC7 vector comprising a fusion protein comprising the amino acid sequence of SEQ ID NO: 187, or an immunogenic fragment thereof. In some embodiments, the amino acid sequence of SEQ ID NO: 187, or an immunogenic fragment thereof, does not contain the N-terminal 25 amino acid signal peptide.

The methods can comprise providing to the subject an effective amount of a vaccine comprising an AdC7 vector, wherein the AdC7 vector comprises a fusion protein comprising the amino acid sequence of SEQ ID NO: 185 or 187, or an immunogenic fragment thereof. In some embodiments, the methods further comprise providing to the subject, subsequent to providing the vaccine comprising the AdC7 vector, a vaccine comprising an AdC6 vector comprising a fusion protein comprising the amino acid sequence of SEQ ID NO: 185 or 187, or an immunogenic fragment thereof. Such prime-boost methods can comprise:
Providing to the subject a vaccine comprising an AdC7 vector comprising a fusion protein comprising the amino acid sequence of SEQ ID NO: 185, or an immunogenic fragment thereof, and subsequently providing to the subject a vaccine comprising an AdC6 vector comprising a fusion protein comprising the amino acid sequence of SEQ ID NO: 185, or an immunogenic fragment thereof. In some embodiments, the amino acid sequence of SEQ ID NO: 185, or an immunogenic fragment thereof, does not contain the N-terminal 25 amino acid signal peptide; or
Providing to the subject a vaccine comprising an AdC7 vector comprising a fusion protein comprising the amino acid sequence of SEQ ID NO: 187, or an immunogenic fragment thereof, and subsequently providing to the subject a vaccine comprising an AdC6 vector comprising a fusion protein comprising the amino acid sequence of SEQ ID NO: 187, or an immunogenic fragment thereof. In some embodiments, the amino acid sequence of SEQ ID NO: 187, or an immunogenic fragment thereof, does not contain the N-terminal 25 amino acid signal peptide.

The immune response induced by the disclosed methods include, but is not limited to, T cell responses, B cell responses, or both (i.e. cellular and/or humoral immune responses). The immune response can be a primary immune response or a secondary immune response. The disclosed methods can induce a subject's immune response against HBV to a greater extent compared to the HBV Core or polymerase antigens alone (i.e. without gD).

The disclosed methods can be used for both therapeutic treatment and prophylactic or preventative measures and can reduce the severity and/or frequency of symptoms, eliminate symptoms and/or the underlying cause of the symptoms, reduce the frequency or likelihood of symptoms and/or their underlying cause, and improve or remediate damage caused, directly or indirectly, by HBV. Treatment also includes prolonging survival as compared to the expected survival of a subject not receiving treatment. Subjects to be treated include those that have HBV as well as those prone to have HBV or those in which HBV is to be prevented.

The amount of the disclosed fusion proteins, nucleic acid molecules, vectors, or vaccines needed to thereby induce an immune response to HBV (e.g. a “effective amount”) may vary according to factors such as the disease state, age, sex, and weight of the subject, and the ability of the fusion proteins, nucleic acid molecules, vectors, or vaccines to cause a desired response in the subject. Exemplary indicators of an effective amount include, for example, improved well-being of the subject and reduction, elimination, or prevention of HBV symptoms.

Also provided is the use of any of the disclosed fusion proteins, nucleic acid molecules, vectors, or vaccines in the manufacture of a medicament for inducing an immune response to HBV in a subject.

The disclosed fusion proteins, nucleic acid molecules, vectors, or vaccines for use in inducing an immune response to HBV in a subject is also provided.

### EXAMPLES

The following examples are provided to further describe some of the embodiments disclosed herein. The examples are intended to illustrate, not to limit, the disclosed embodiments.

### Generation of an Epitope-Optimized Core Sequence

Hepatitis B virus (HBV) can be grouped into several genotypes, based on phylogenic clustering. To assist in the development of an antigen insert for a multi-genotype HBV vaccine for patients with chronic infections, a preliminary bioinformatics evaluation of the genes encoding the HBV Core and HBV polymerase across genotypes A, B, C and D was conducted.

The Core amino acid sequences from the four major HBV clades were downloaded as aligned ClustalW sequences from Hepatitis B Virus database (HBVdb) (release version 45.0; last updated on Aug. 2, 2018). The amino acid sequences represented thousands of HBV genomes inputted from users across Europe, as summarized in the following table.

TABLE 1
Number of unique Core genomes analyzed

| Genotype | HBV Gene | Unique Genomes Analyzed |
|---|---|---|
| HBV genotype A | Core | 1,482 |
| HBV genotype B | | 2,800 |
| HBV genotype C | | 2,768 |
| HBV genotype D | | 1,579 |

“Consensus” Core sequences were first identified for each genotype using the Shannon Entropy tool hosted by the Los Alamos National Laboratory (www.hiv.lanl.gov/content/sequence/ENTROPY/entropy), which calculated the variation and frequency at each amino acid position. These calculations were repeated for each genotype, generating four “consensus” Core sequences, one for each genotype analyzed (SEQ ID NOs: 1-4):

Genotype A Consensus
(SEQ ID NO: 1)
MDIDPYKEFGATVELLSFLPSDFFPSVRDLLDTAS
ALYREALESPEHCSPHHTALRQAILCWGELMTLAT
WVGNNLeDPASRDLVVNYVNTNMGLKIRQLLWFHI
SCLTFGRETVLEYLVSFGVWIRTPPAYRPPNAPIL
STLPETTVVRRRDRGRSPRRRTPSPRRRRSQSPRR
RRSQSRESQC-
Genotype B Consensus
(SEQ ID NO: 2)
MDIDpYKEFGASvELLSFLPSDFFPSiRDLLDTAs
ALYREALESPEHCSPHHTALRQAIlCWGELMNLAT
WVGSNLeDPASRELVVsYVNVNMGLKiRQLLWFHI
SCLTFGRETVLEYLVSFGVWIRTPpAYRPpNAPIL
STLPETTVVRRRGRSPRRRIPSPRRRRSQSPRRRR
SQSREsQC-
Genotype C Consensus
(SEQ ID NO: 3)
MDIDpYKEFGASVELLSFLPSDFFPSIRDLLDTAS
ALYREALESPEHCSPHHTALRQAILCWGELMNLAT
WVGSNLEDPASRELVVsYVNVNMGLKiRQlLWFHI
SCLTFGRETVLEYLVSFGVWIRTPpAYRPPNAPIL
STLPETTVVRRRGRSPRRRIPSPRRRRSQSPRRRS
QSRESQC -
Genotype D Consensus
(SEQ ID NO: 4)
MDIDPYKEFGAtVELLSFLPsDFFPSVRDLLDTAS
ALYReALESPEHCSPHHTALRQAILCWGeLMtLAT
WVGgNLEDPaSRDLVVSYVNTNmGLKFRQLLWFHI
SCLTFGReTViEYLVSFGVWIRTPpAYRPPNAPIL
STLPETTVvRRRGRSPRRRIPSPRRRTSQSPRRRR
SQSRESQC-
(Bold, underlined residues represent amino
acids having less than 90% frequency).

(Bold, underlined residues represent amino acids having less than 90% frequency).

The above “consensus” Core sequences were combined to generate an epitope-optimized Core sequence. Conserved amino acids were identified at each amino acid residue of the Core protein from each genotype (A, B, C and D) and the frequency and variation within a given sample of genotype genomes was determined. To select amino acids at sites of variation, each variation was tested using epitope prediction algorithms across multiple HLA types and the most immunogenic sequence was selected. Specifically:
(1) Each residue across the four genotypes that was identical were maintained. The genome weighted frequency was also calculated to inform the variability with spacer added, where applicable, to align the sequences for diversity.
(2) Residues that were not identical across the four genotypes were identified and the amino acid diversity was recorded (see Table 2). The initial Core sequence (SEQ ID NO: 5) is provided below, with the residues that were not identical across the four genotypes labeled as X₁-X₁₁ and the residues having less than 90% frequency in bold, underlined font:

MDIDPYKEFGAX₁VELLSFLPSDFFPSX₂DLLDTASALYREALE
SPEHCSPHHTALRQAILCWGELMX₃LATWVGX₄NLeDPASRX₅L
VVX₆YVNX₇NMGLKX₈RQLLWFHISCLTFGRETVX₉EYLVSFGV
WIRTPPAYRPPNAPILSTLPETTVVRRRX₁₀X₁₁GRSPRRRITS
PRRRRSQSPRRRRSQSRESQC

TABLE 2
Residues that were not identical across the four genotypes

| Residue # | X₁ | X₂ | X₃ | X₄ | X₅ | X₆ | X₇ | X₈ | X₉ | X₁₀ | X₁₁ |
|---|---|---|---|---|---|---|---|---|---|---|---|
| Genotype A | T | V | T | N | D | N | T | I | L | D | R |
| Consensus | | | | | | | | | | | |
| A - Consensus | 95.4% | 98.2% | 96.0% | 94.0% | 99.5% | 98.9% | 98.3% | 98.8% | 98.2% | 95.8% | 99.5% |
| Frequency | | | | | | | | | | | |
| Genotype B | S | i | N | S | E | s | V | i | L | — | — |
| Consensus | | | | | | | | | | | |
| B - Consensus | 97.1% | 89.7% | 93.5% | 90.1% | 91.5% | 75.2% | 93.6% | 80.1% | 99.4% | — | — |
| Frequency | | | | | | | | | | | |
| Genotype C | S | I | N | S | E | s | V | i | L | — | — |
| Consensus | | | | | | | | | | | |
| C - Consensus | 99.4% | 90.3% | 97.3% | 96.3% | 97.0% | 83.7% | 97.8% | 78.7% | 98.8% | — | — |
| Frequency | | | | | | | | | | | |
| Genotype D | t | V | t | g | D | S | T | F | i | — | — |
| Consensus | | | | | | | | | | | |
| D - Consensus | 75.7% | 97.0% | 87.5% | 58.7% | 98.4% | 93.7% | 96.5% | 99.1% | 77.8% | — | — |
| Frequency | | | | | | | | | | | |

(3) To determine the final amino acid at these positions, epitope prediction algorithms were used to select the appropriate amino acid. For amino acids that showed variability between the genotypes, amino acids that were present in 3 of the genotypes were selected or an MHC class I epitope prediction software was used to select the most immunogenic amino acids. This approach maximized the potential immunogenicity across the greatest number of HLA types. The epitope-optimized Core sequence across all genotypes and within genotypes is shown below (SEQ ID NO: 6):

DIDPYKEFGATVELLSFLPSDFFPSIRDLLDTASALYREALESP
EHCSPHHTALRQAILCWGELMTLATWVGSNLEDPASRELVVSYV
NVNMGLKIRQLLWFHISCLTFGRETVIEYLVSFGVWIRTPPAYR
PPNAPILSTLPETTVVRRRDRGRSPRRRTPSPRRRRSQSPRRRR
SQSRESQC

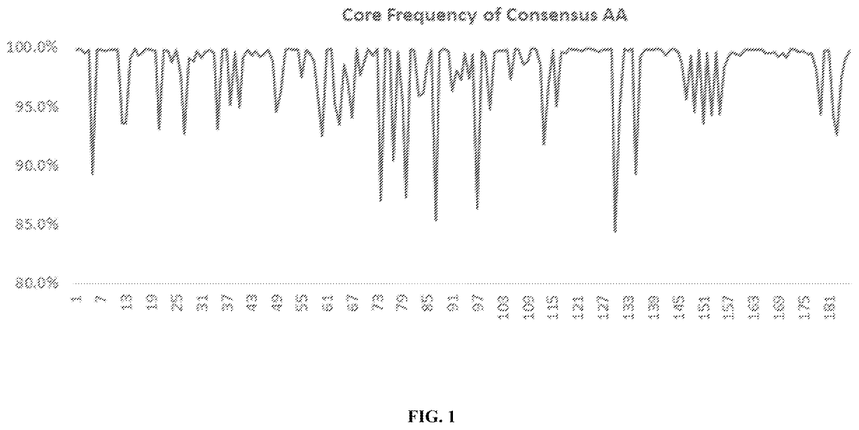

The average variation at each site across all genomes, weighted by the number of Glade-specific genomes analyzed, was calculated and showed areas and residues of higher and greater conservation. FIG. 1.

### Generation of Epitope-Optimized Polymerase Sequences

An epitope-optimized polymerase sequence was generated from the four major HBV clades as discussed above for the Core sequence. Because the polymerase is long, two fragments—an N-terminal fragment (from which a highly variable segment between the genotypes was removed) and a C-terminal fragment—were generated. Both fragments are approximately 300 amino acids in length. The epitope-optimized polymerase amino acid sequences are shown below and in Table 9:

Epitope-optimized HBV polymerase N-terminal
amino acid sequence
(SEQ ID NO: 8):
PLSYQHFRKLLLLDEEAGPLEEELPRLADEGLNRRVAEDL
NLGNLNVSIPWTHKVGNFTGLYSSTVPVFNPEWQTPSFPK
IHLQEDIVDRCKQFVGPLTVNEKRRLKLIMPARFYPNVTK
YLPLDKGIKPYYPEHAVNHYFQTRHYLHTLWKAGILYKRE
TTRSASFCGSPYSWEQELQHGSCWWLQFRNSKPCSEYCLT
HLVNLLEDWGPCDEHGEHHIRIPRTPARVTGGVFLVDKNP
HNTAESRLVVDFSQFSRGITRVSWPKFAVPNLQSLTNLLS
SNLSWLSLDVSAAFYHIPLHPAAMP
Epitope-optimized HBV polymerase C-terminal
amino acid sequence
(SEQ ID NO: 10):
HLLVGSSGLSRYVARLSSNSRIINHQHGTMQNLHDSCSRN
LYVSLLLLYKTFGRKLHLYSHPIILKTKRWGYSLNFMGYV
IGSWGSLPQDHIIQKIKECFRKLPVNRPIDWKVCQRIVGL
LGFAAPFTQCGYPALMPLYACIQSKQAFTFSPTYKAFLSK
QYLNLYPVARQRPGLCQVFADATPTGWGLAMGHQRMRGTF
VAPLP1HTAELLAACFARSRSGAKILGTDNSVVLSRKYTS
FPWLLGCAANWILRGTSFVYVPSALNPADDPSRGRLGLSR
PLLRLPFRPTTGRTSLYAVSPSV

### Generation of AdC6 and AdC7 Vectors Expressing the Epitope-Optimized Core and Polymerase Sequences

The genes encoding the epitope-optimized Core or polymerase amino acid sequences were cloned into transfer vectors that contained the herpes simplex virus (HSV) glycoprotein D (gD) sequence under the control of the CMV promoter. The genes were then cloned into the El-deleted, E3 ORF 3, 4, 5, 6, and 7-deleted replication deficient adenoviral vector (as described in PCT/US2017/043315) to generate the following vectors:
AdC6 containing the epitope-optimized Core sequence fused to gD (AdC6-gDCore);
AdC6 containing the epitope-optimized polymerase N-terminal sequence fused to gD (AdC6-gDPolN);
AdC6 containing the epitope-optimized polymerase C-terminal sequence fused to gD (AdC6-gDPolC);
AdC7 containing the epitope-optimized Core sequence fused to gD (AdC7-gDCore);
AdC7 containing the epitope-optimized polymerase N-terminal sequence fused to gD (AdC7-gDPolN); and
AdC7 containing the epitope-optimized polymerase C-terminal sequence fused to gD (AdC7-gDPolC).

Correct clones were identified by restriction enzyme digest and the cloning sites were sequenced. Vectors were rescued and expanded in HEK 293 cells, purified by cesium chloride (CsC1) gradient centrifugation, and the vector concentration (vp) was determined by spectrophotometry. Vectors were titrated for infectious units upon their expansion in serial dilutions in HEK 293 cells, followed by isolation and reverse transcription of RNA and a nested hexon-specific PCR reaction. Genetic integrity of the vectors was determined by restriction enzyme digest followed by gel electrophoresis of purified viral DNA. Protein expression was determined by Western blotting using gD-specific antibodies. Genetic stability was determined by serial passages (12-15) of the vectors in HEK 293 cells followed by restriction enzyme digest of purified viral DNA and gel electrophoresis.

### Testing of Immunogenicity of Vaccines in Mice

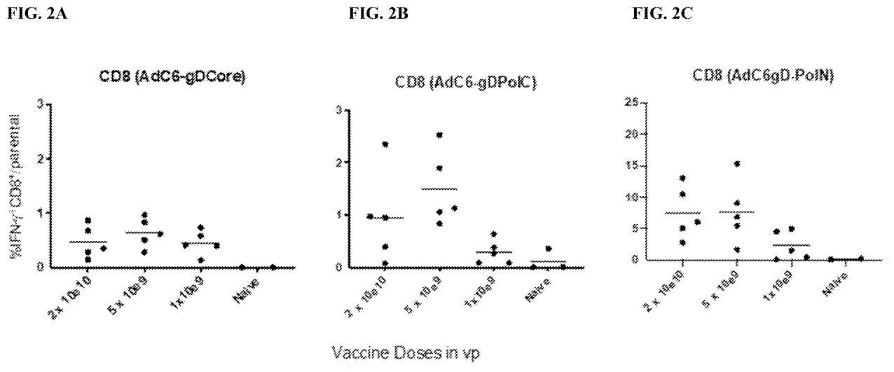

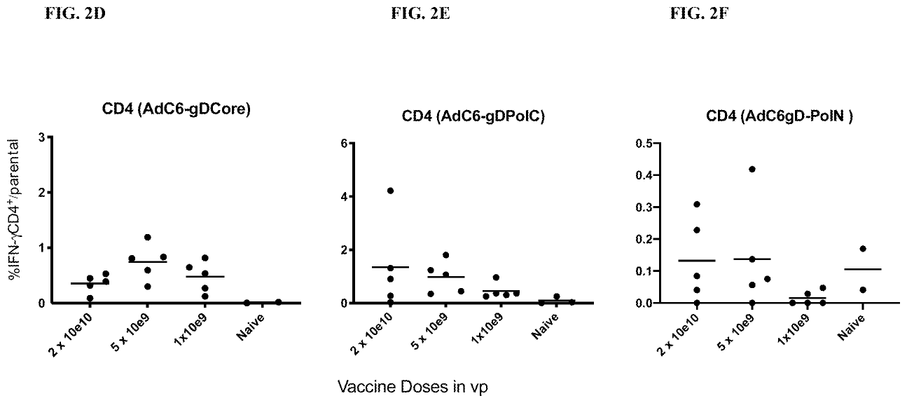

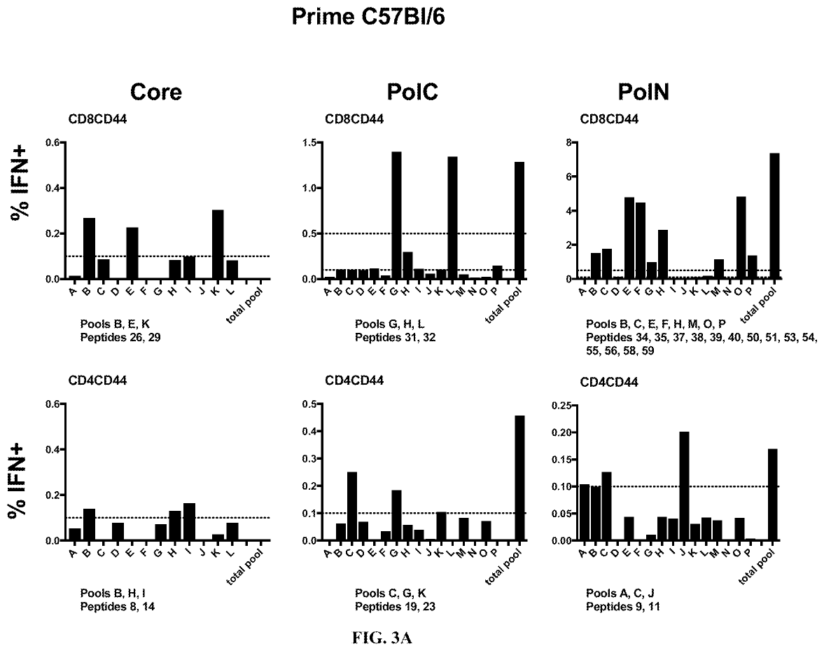

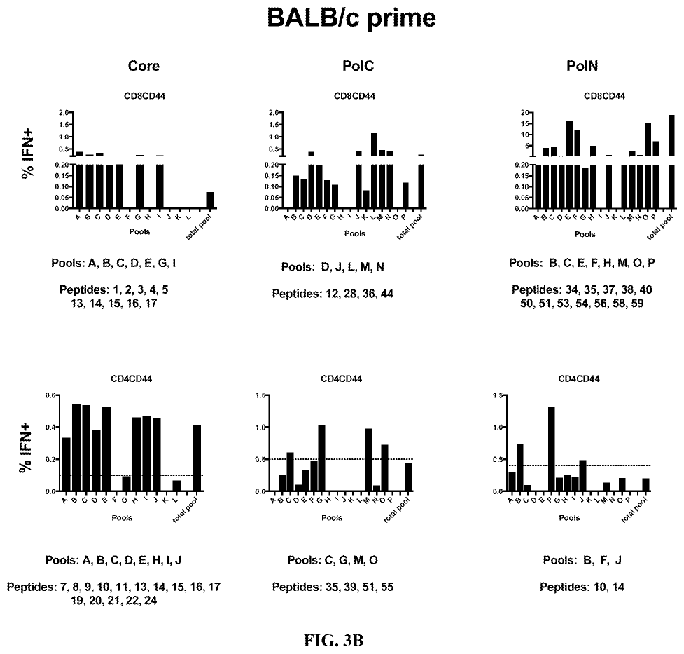

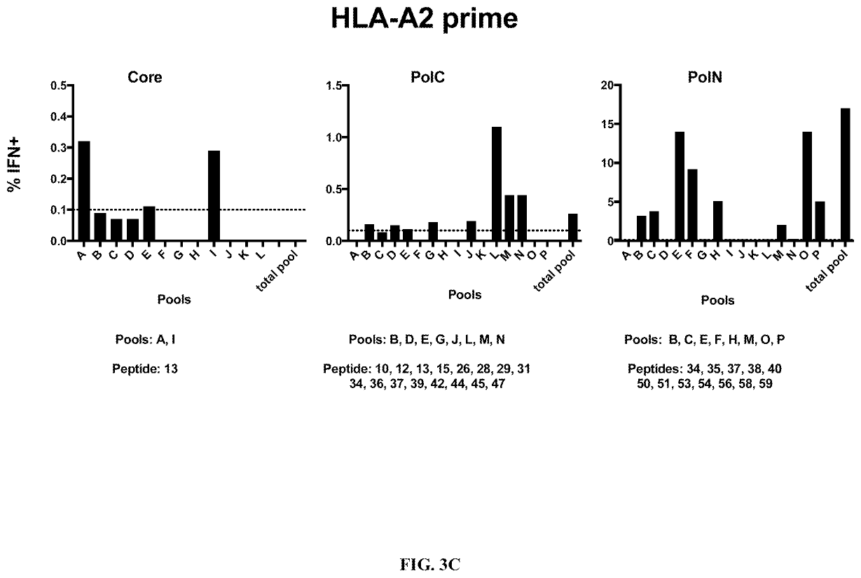

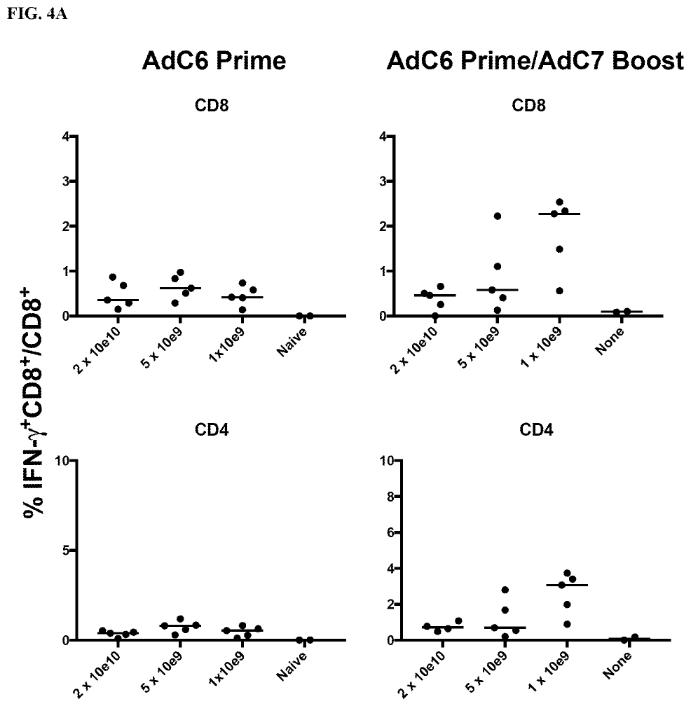

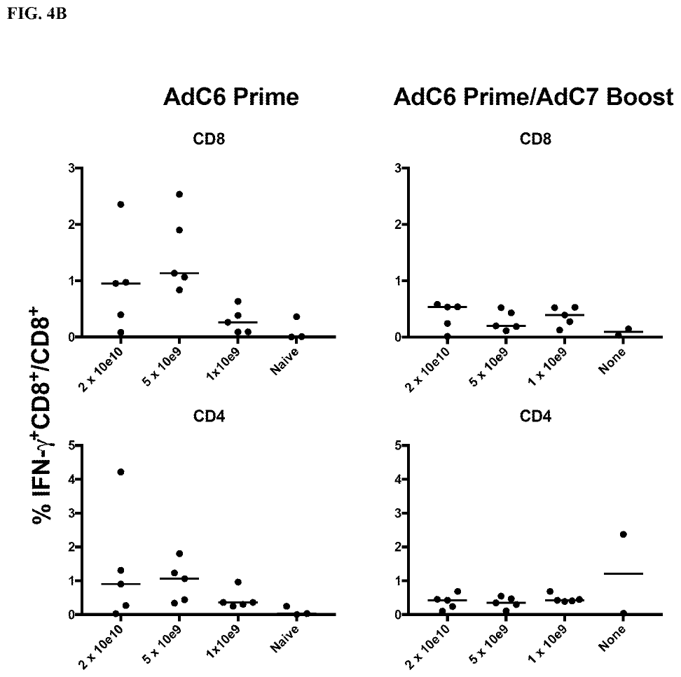

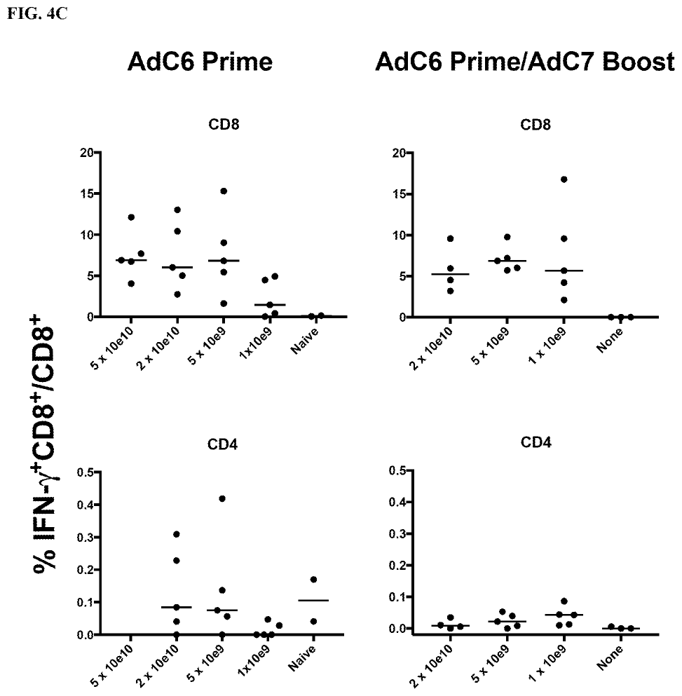

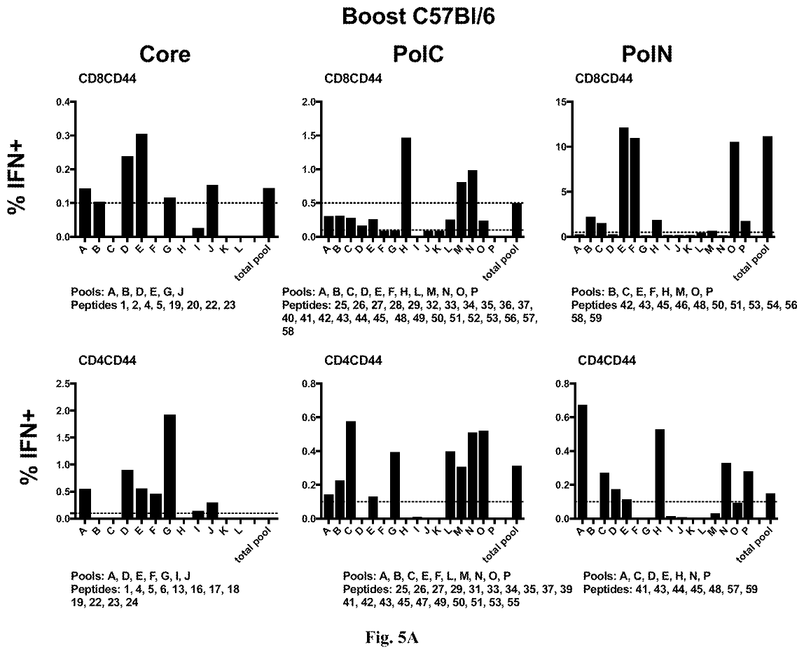

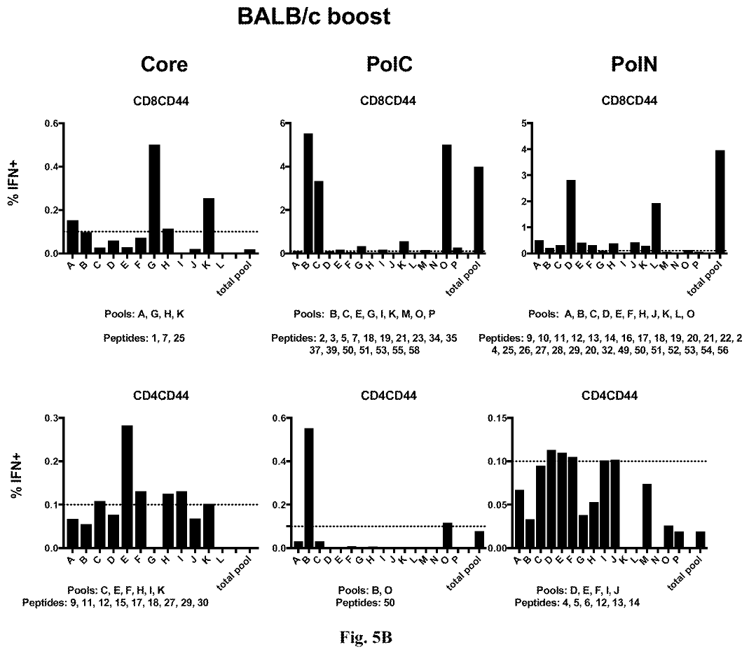

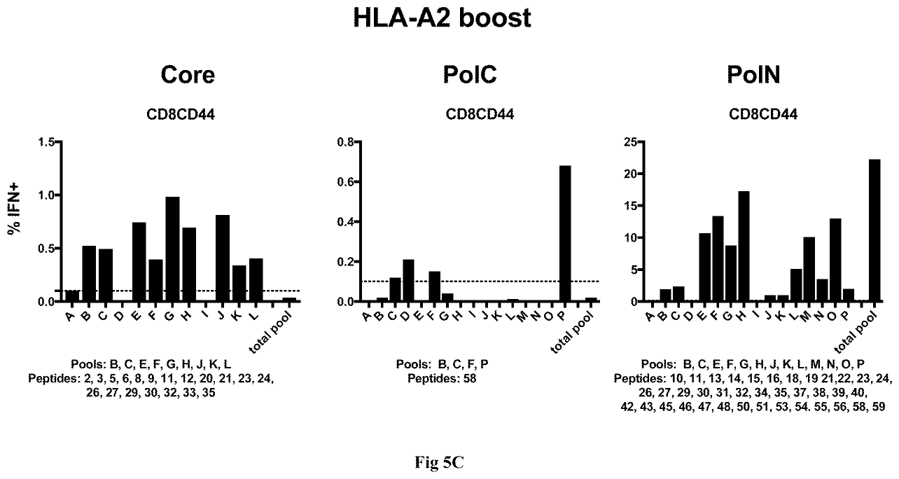

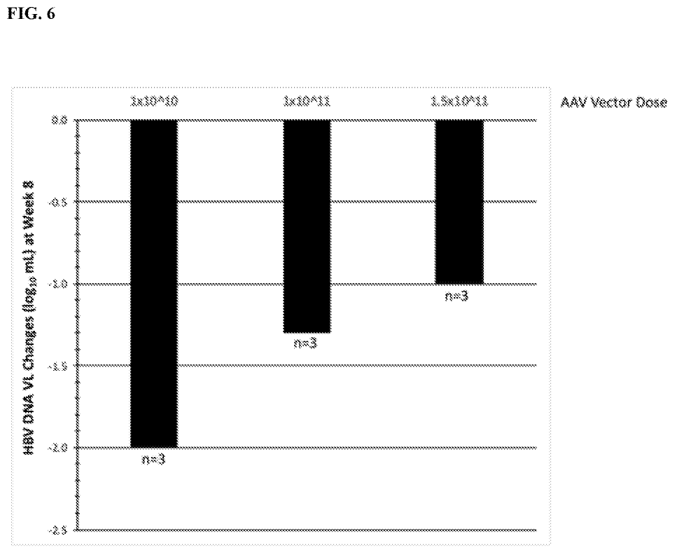

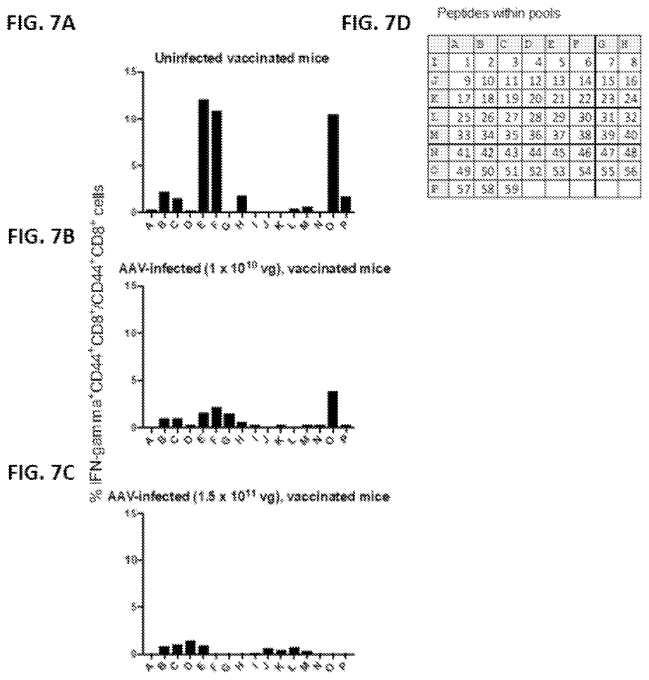

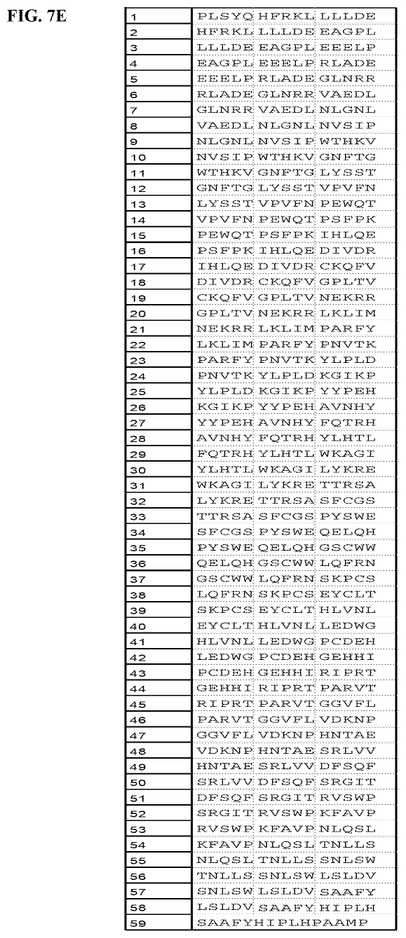

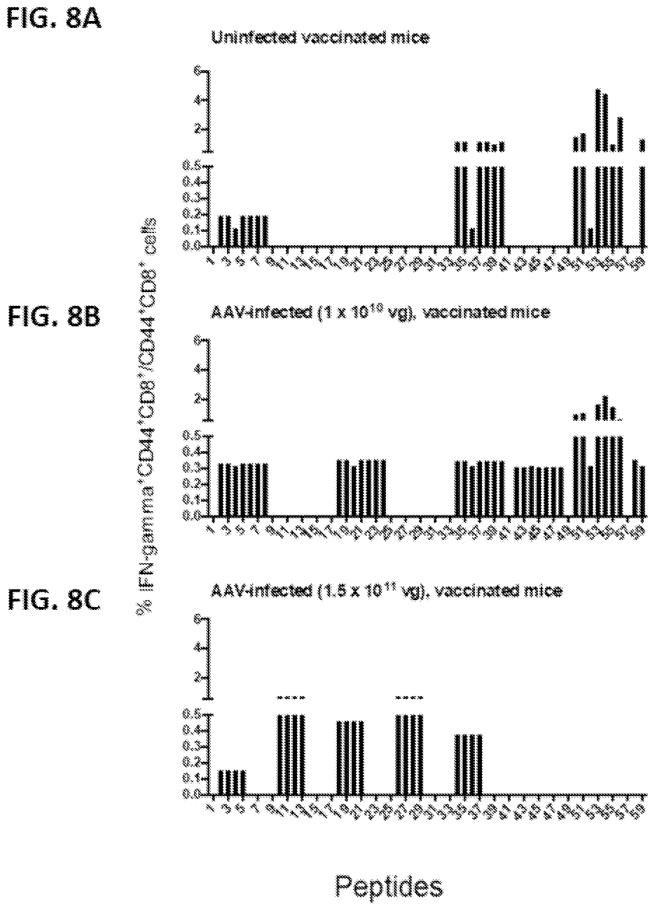

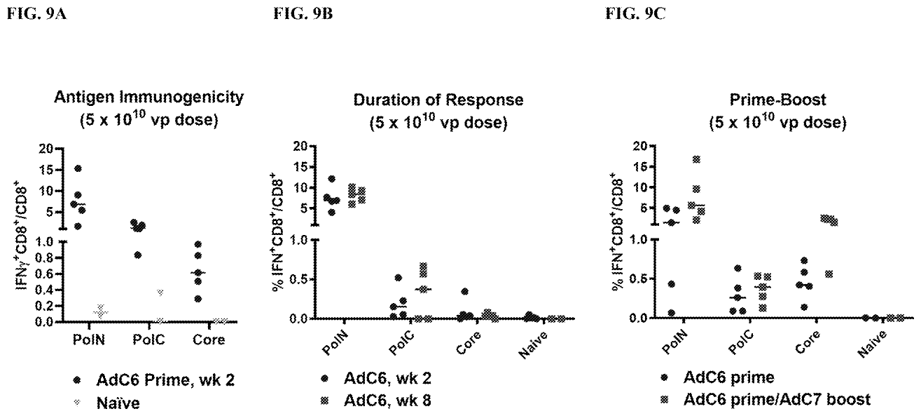

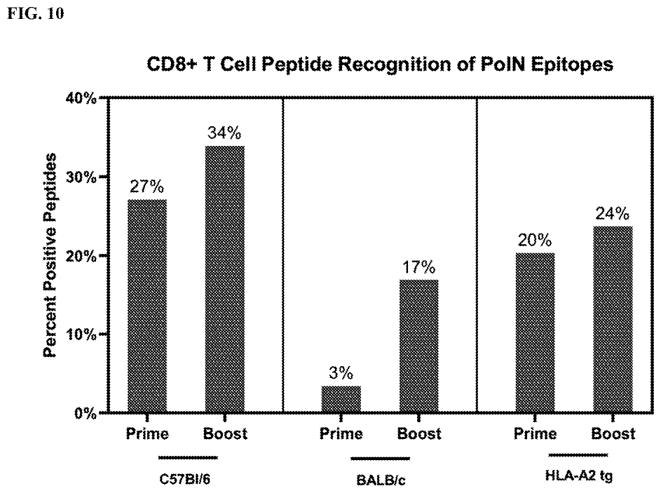

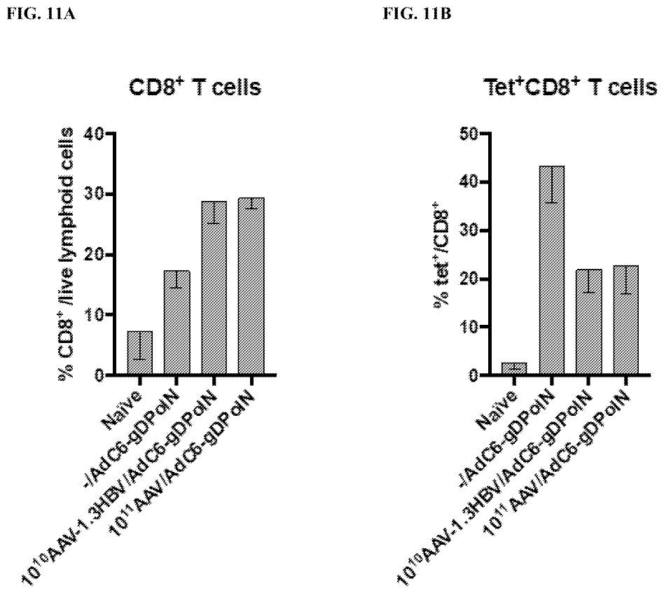

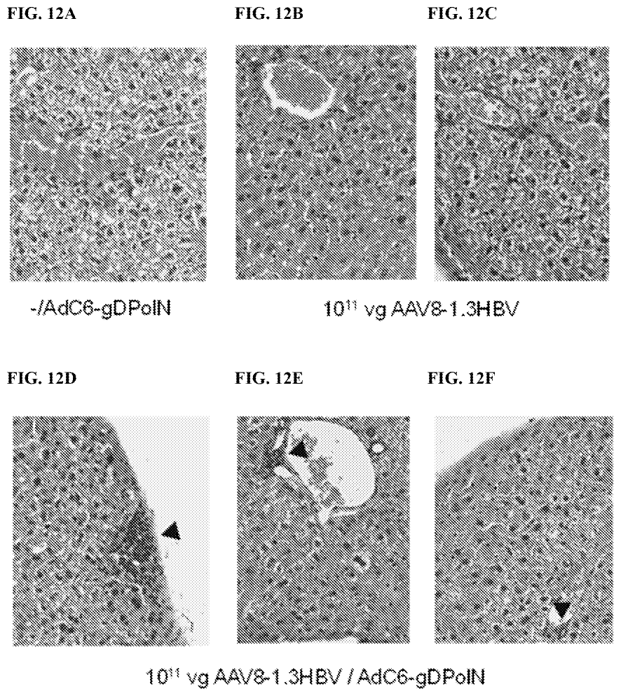

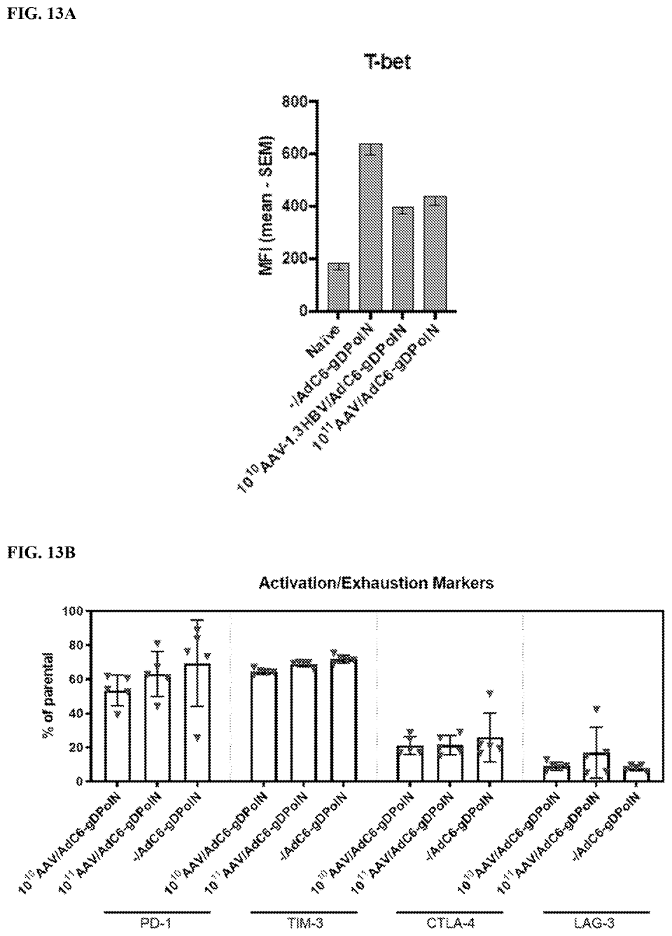

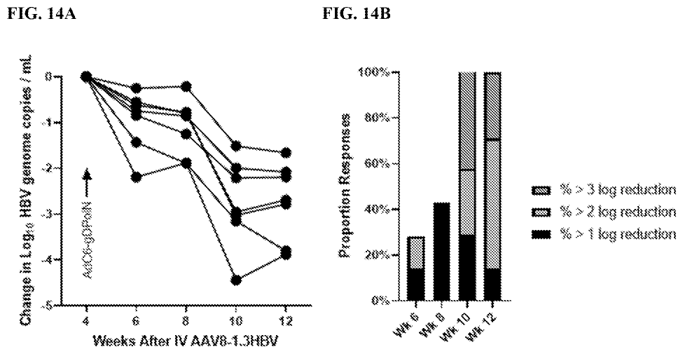

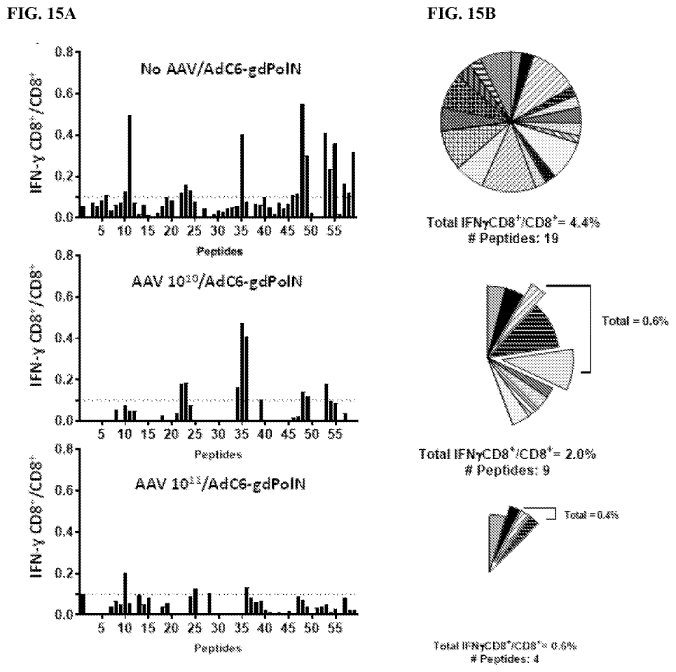

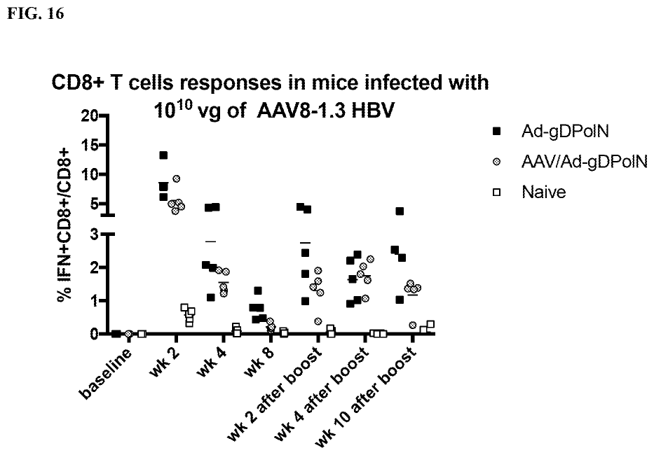

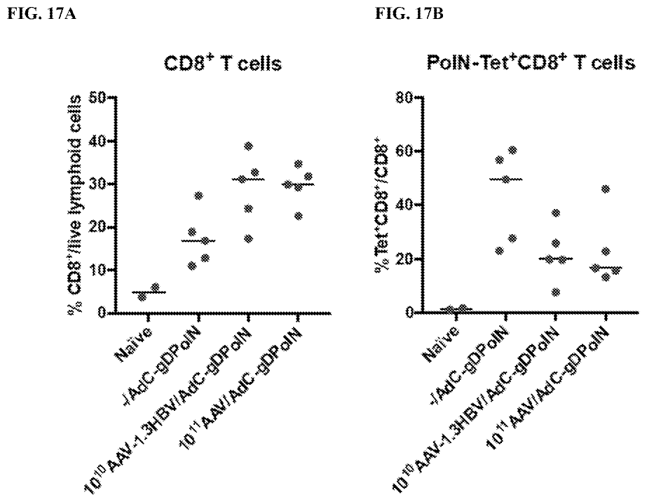

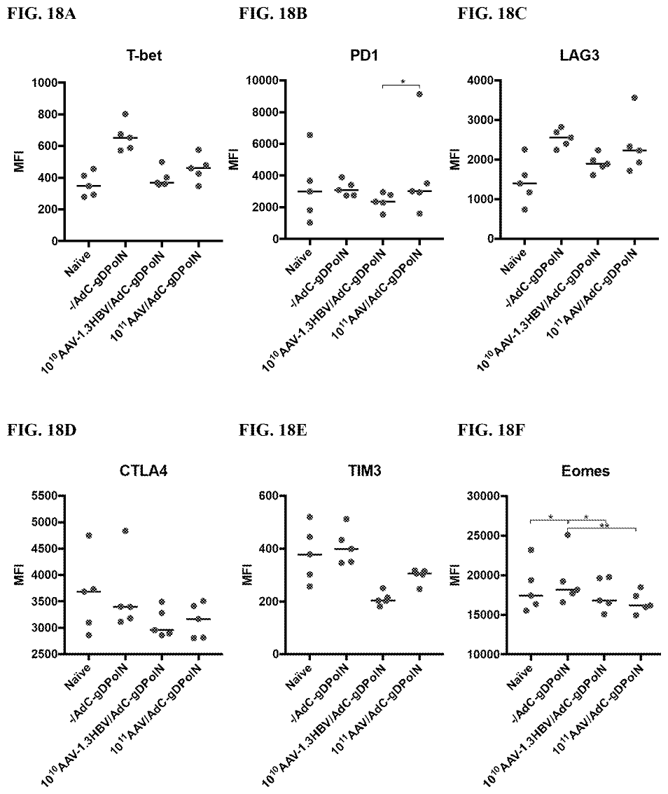

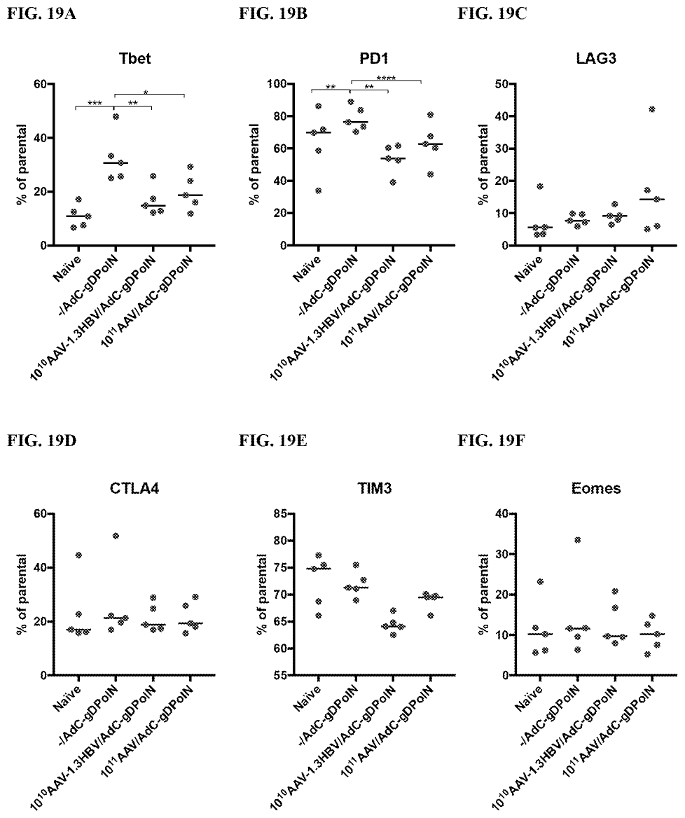

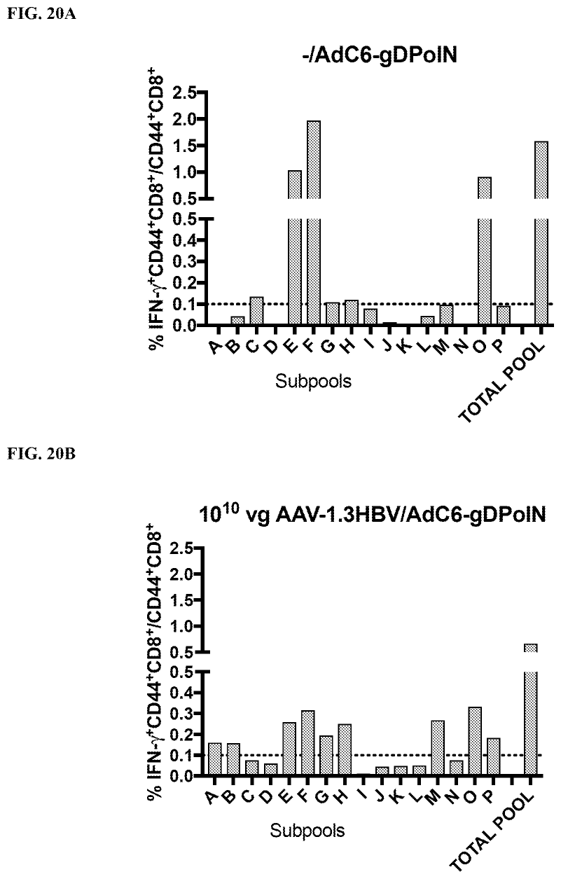

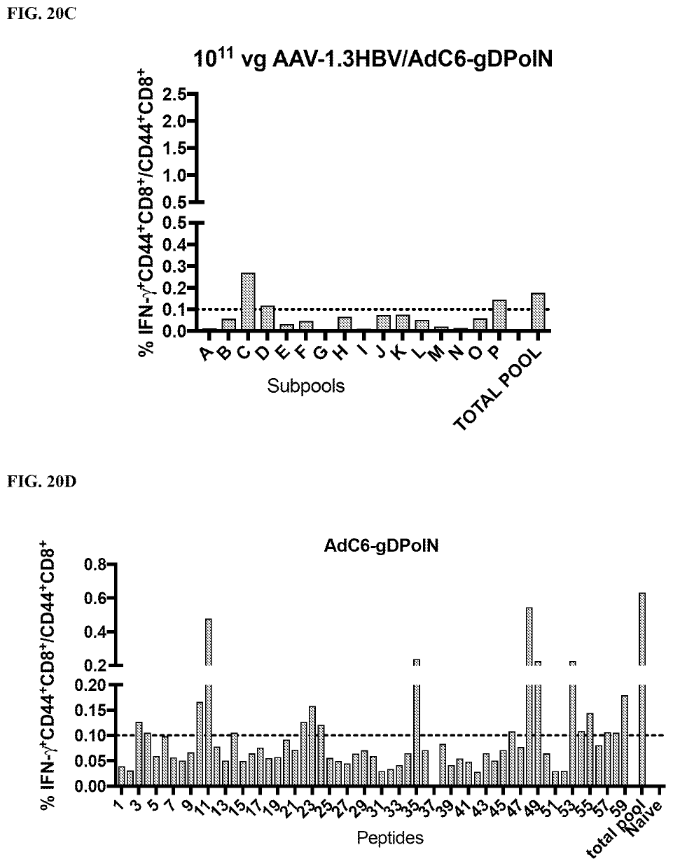

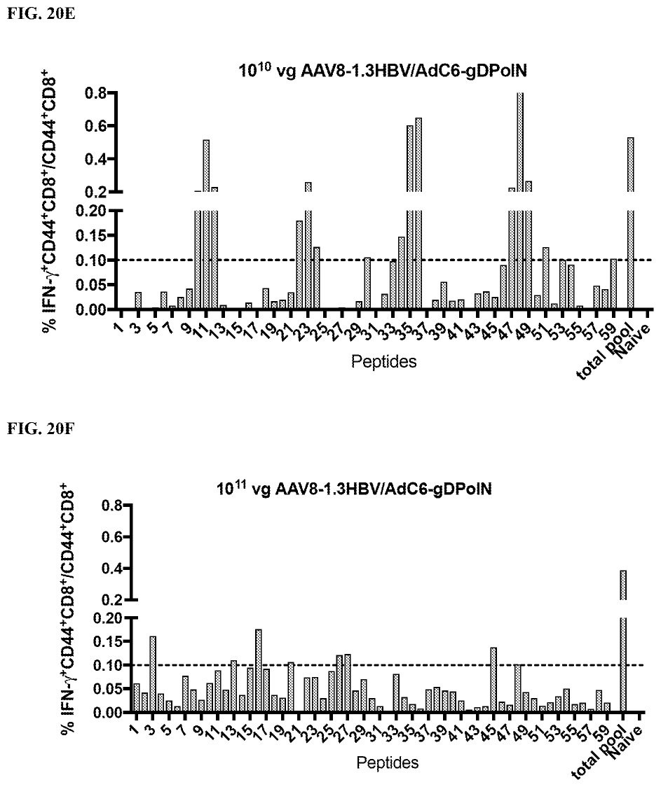

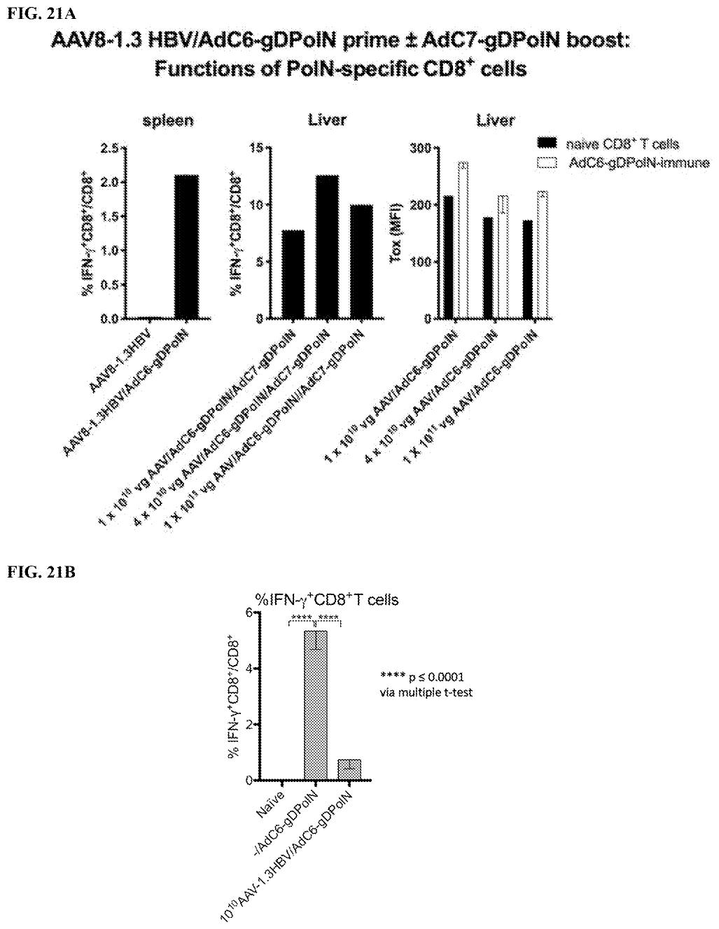

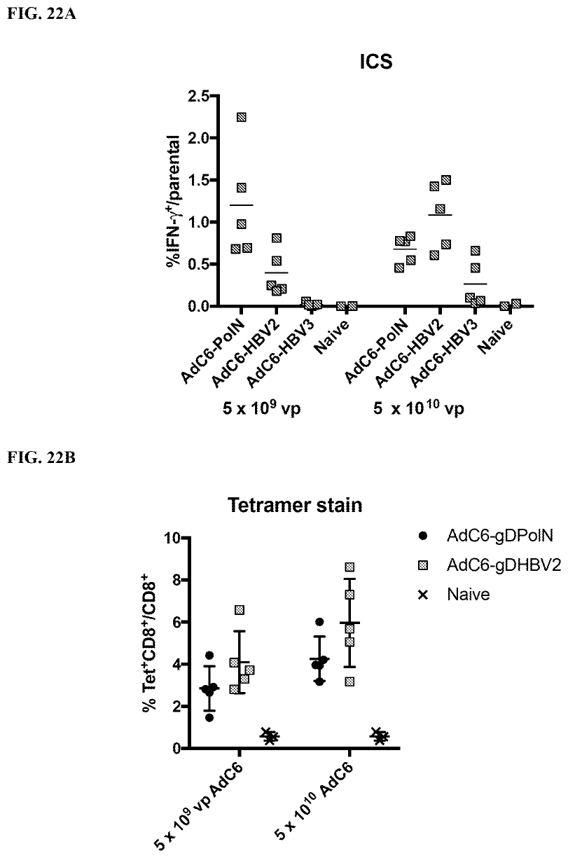

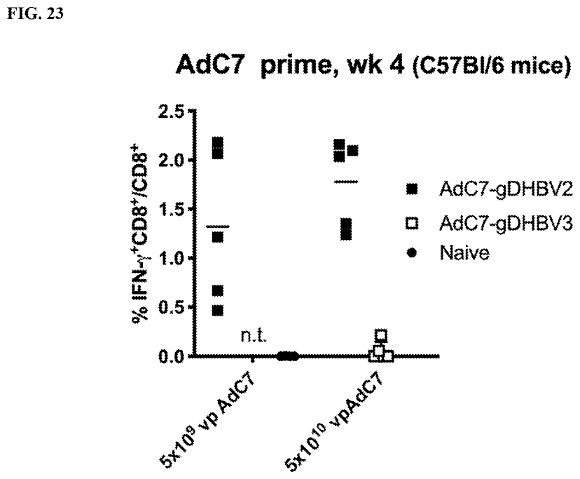

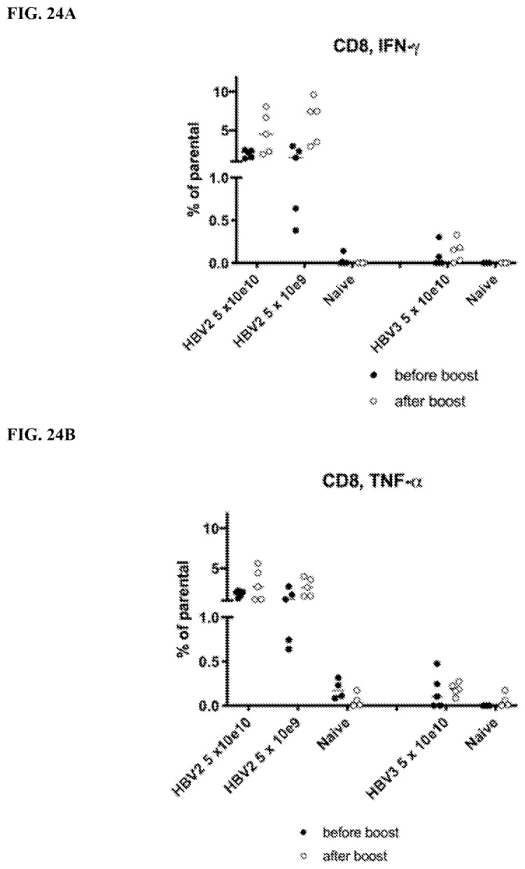

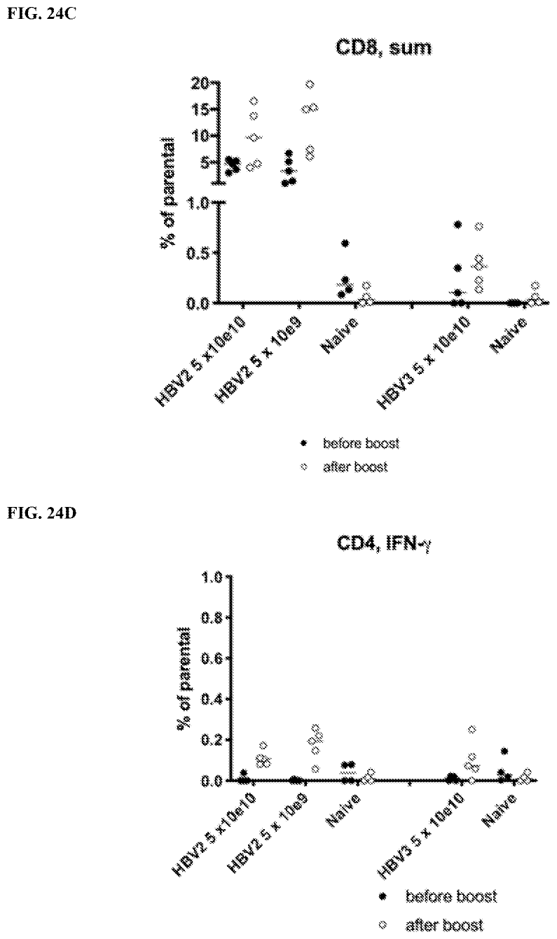

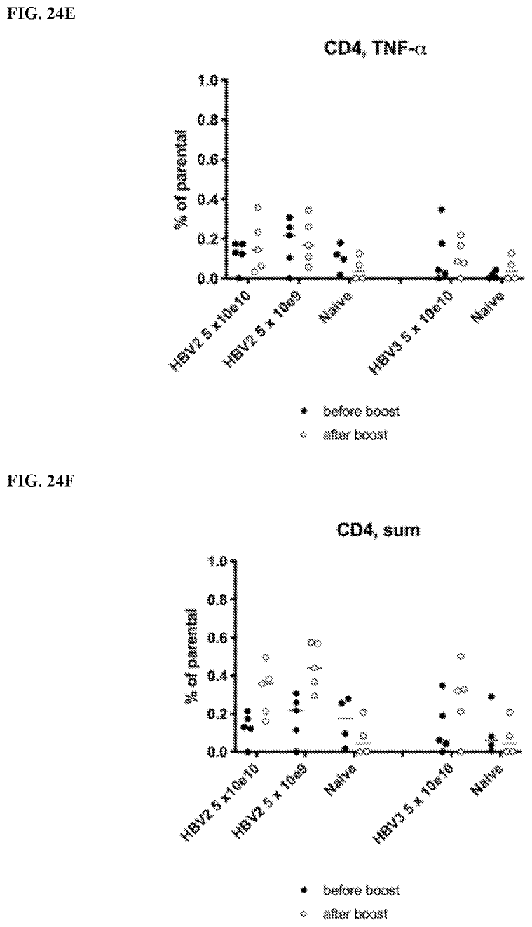

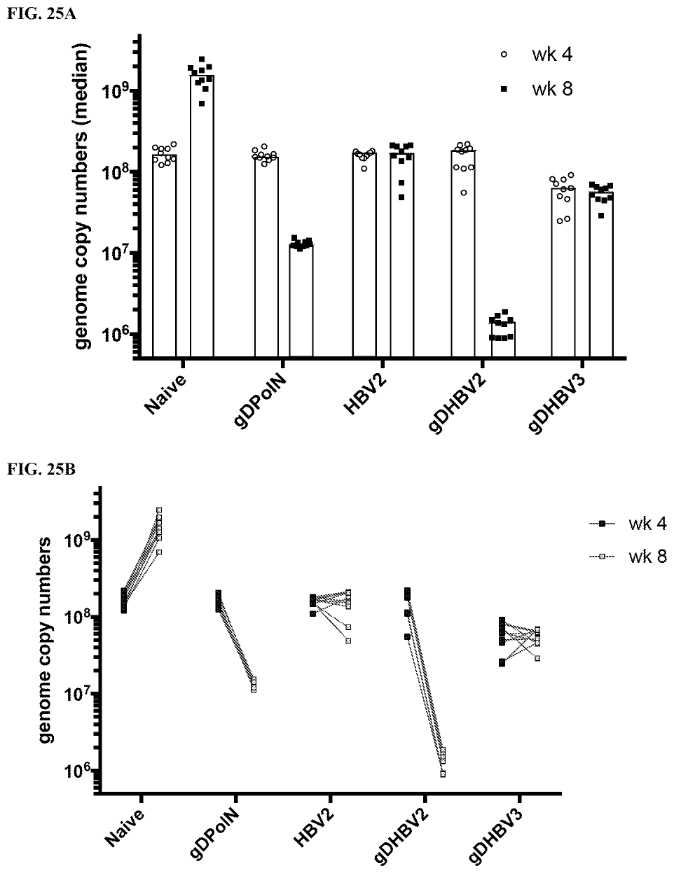

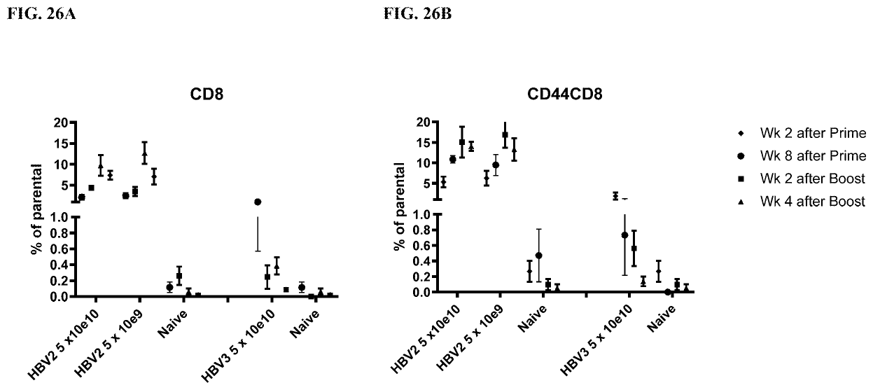

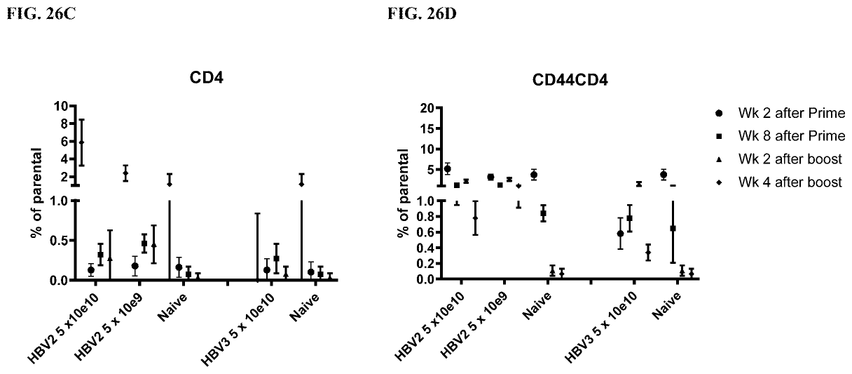

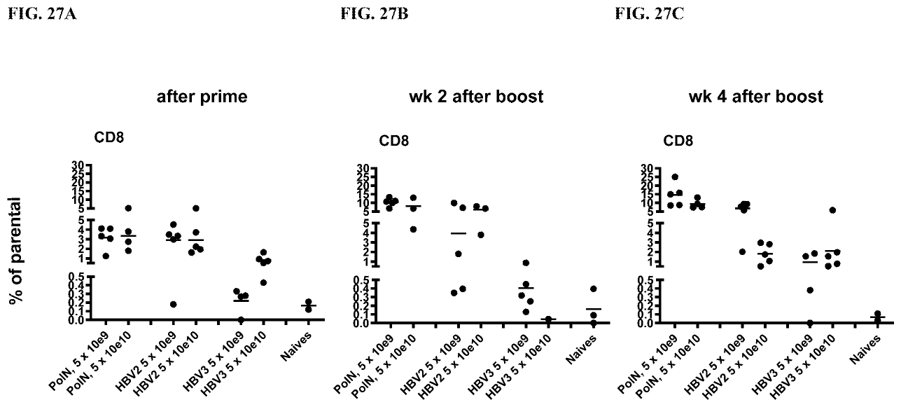

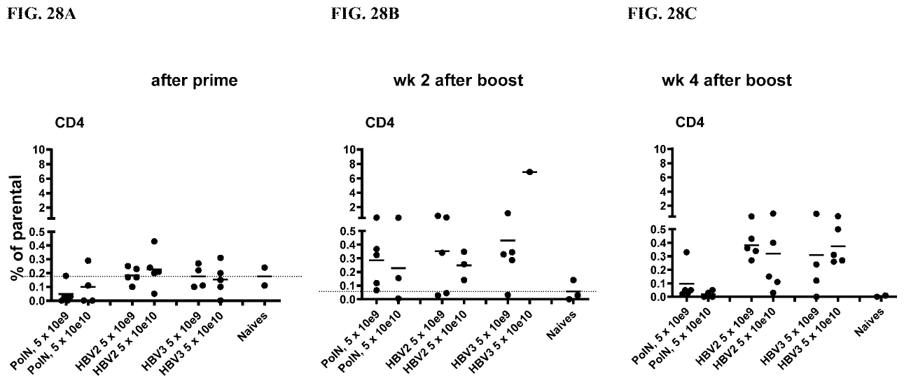

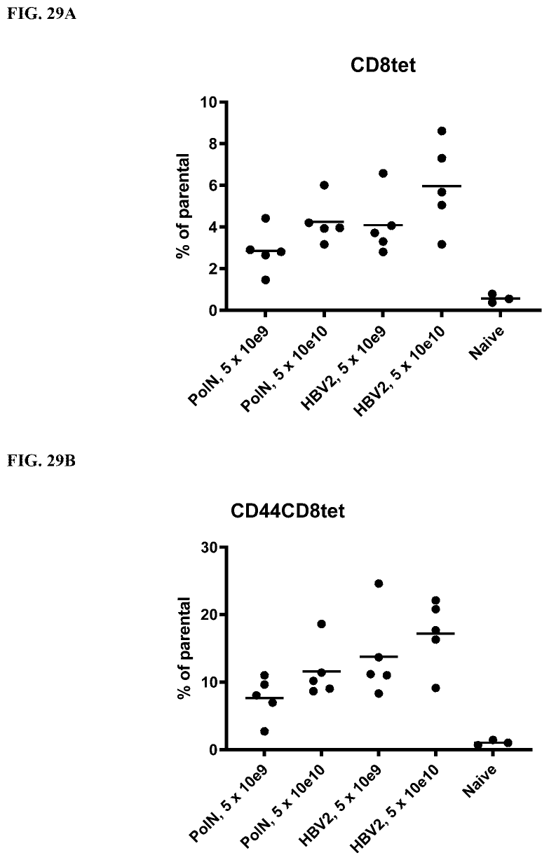

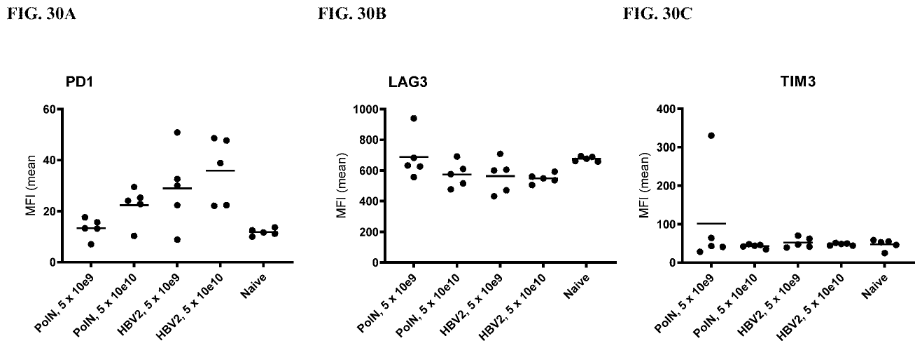

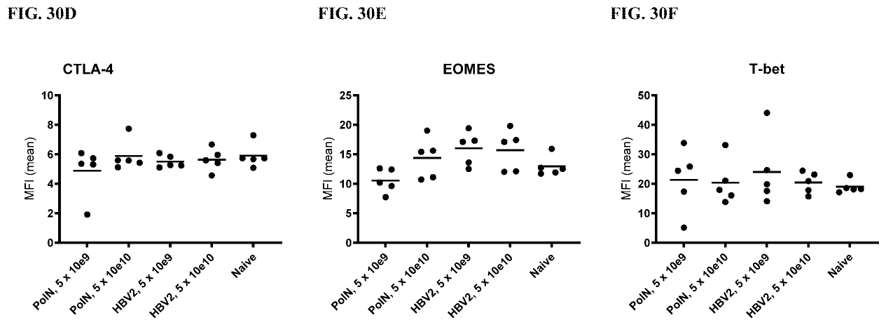

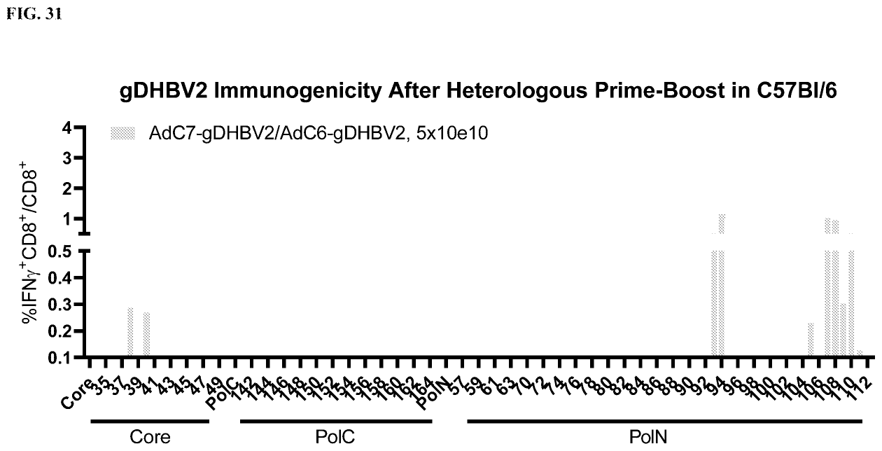

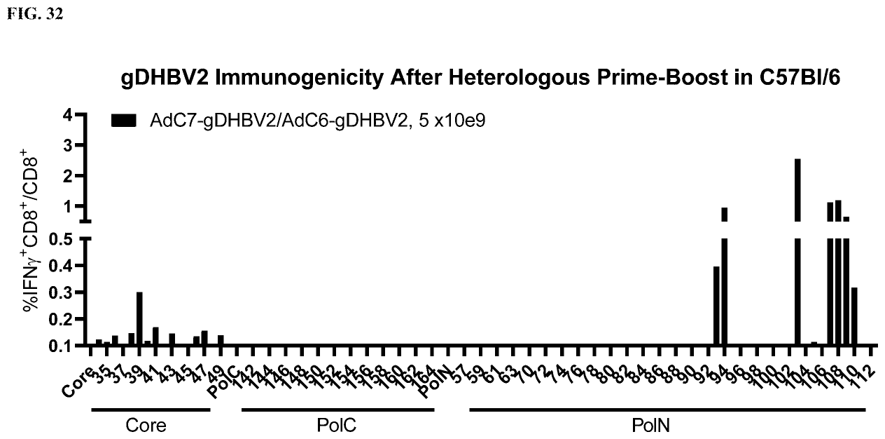

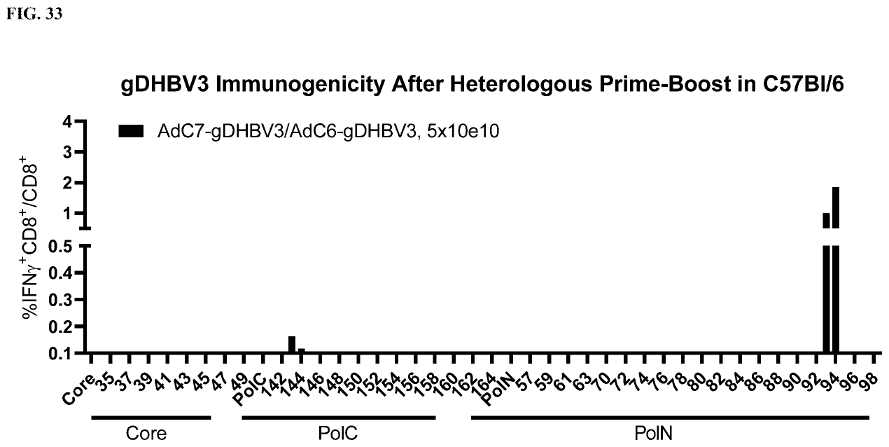

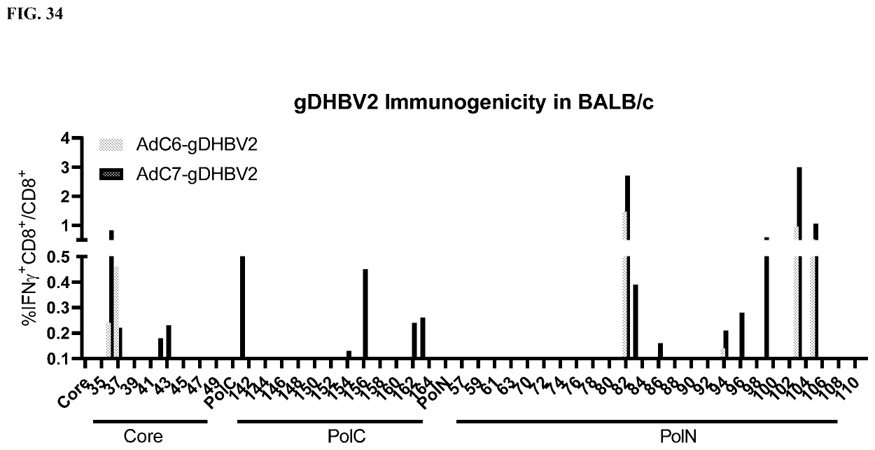

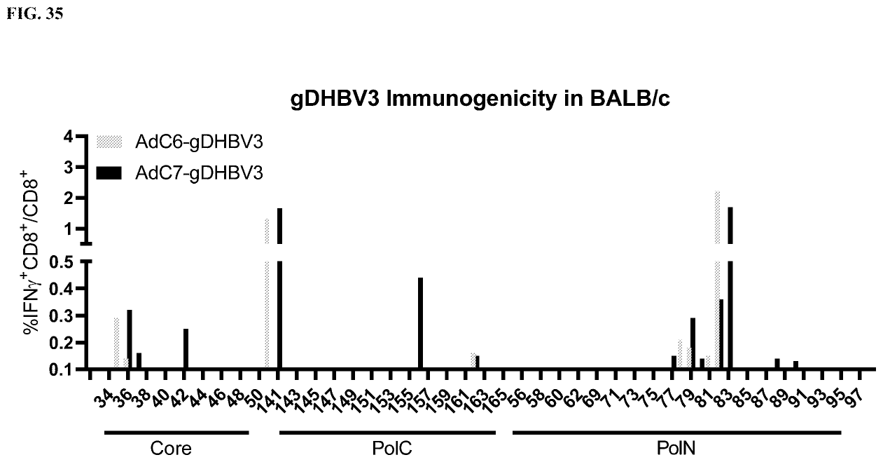

C57B1/6, BALB/c, and HLA-A2 tg mice (n=5 per group) were injected with various concentrations of each of the above vectors. Naïve mice served as controls. Mice were bled at different times after the injection and frequencies of insert-specific CD8+and CD4+ T cells were determined by intracellular cytokine staining (ICS) for IFN-γ. Two months after the first injection, AdC6-immune mice were boosted with the heterologous vector (AdC7) expressing the same insert. Frequencies of HBV-specific T cells were tested again. Results after priming are shown in FIG. 2A-2F and FIG. 3A (C57B1/6 mice), FIG. 3B (BALB/c mice) and FIG. 3C (HLA-A2 mice). Results after the boost are shown in FIGS. 4A-4C and 5A-5B.

C57B1/6 mice showed a very robust CD8+ T cell response to the epitope-optimized polymerase N-terminal sequence and lower responses to the epitope-optimized polymerase C-terminal sequence and the epitope-optimized Core sequence, while CD4+ responses were better against the epitope-optimized Core sequence and the epitope-optimized polymerase C-terminal sequence (FIG. 2A-FIG. 2F). Epitope mapping in C57B1/6 mice showed higher and broader responses to PolN than PolC (FIG. 3A). Within PolN a total of 14 peptides were recognized by CD8+ T cells while within PolC only two adjacent peptides, which most likely reflect one epitope, were recognized. CD4+ T cells failed to respond to PolN or PolC. This pattern was largely mirrored in BALB/c mice, where CD8+ T cell responses were highest against PolN with recognition of 12 peptides followed by PolC with recognition of 4 peptides (FIG. 3B). Responses to Core were low but surprisingly broad with recognition of 10 peptides (FIG. 3B). BALB/c CD4+ T cells responded best to Core with recognition of 15 peptides with lower recognition of PolC (4 peptides) or PolN (2 peptides). CD8+ T cell responses were also tested in HLA-A2 tg mice where PolN again triggered the highest response involving 12 peptides (FIG. 3C). The response to PolC was lower but broader (16 peptides) while only one peptide of Core was detected (FIG. 3C). The sequences of the peptides tested in the priming experiments are provided in Table 3 (Core peptides), Table 4 (PolN peptides), and Table 5 (PolC peptides). The peptide composition of the peptide pools from the priming experiments are provided in Tables 6-8. Overall these data show that the inserts elicited detectable T cell responses that in most cases were directed against multiple epitopes within each sequence.

TABLE 3
Epitope-Optimized Core Peptides

| | | SEQ | |
|---|---|---|---|
| | Amino Acid | ID | |
| Peptide | Sequence | NO: | Prime |
| 1 | DIDPYKEFGATVELL | 20 | CD8 (B/c) |
| 2 | KEFGATVELLSFLPS | 21 | CD8 (B/c) |
| 3 | TVELLSFLPSDFFPS | 22 | CD8 (B/c) |
| 4 | SFLPSDFFPSIRDLL | 23 | CD8 (B/c) |
| 5 | DFFPSIRDLLDTASA | 24 | CD8 (B/c) |
| 6 | IRDLLDTASALYREA | 25 | |
| 7 | DTASALYREALESPE | 26 | CD4 (B/c) |
| 8 | LYREALESPEHCSPH | 27 | CD4 (Bl/6); |
| | | | CD4 (B/c) |
| 9 | LESPEHCSPHHTALR | 28 | CD4 (B/c) |
| 10 | HCSPHHTALRQAILC | 29 | CD4 (B/c) |
| 11 | HTALRQAILCWGELM | 30 | CD4 (B/c) |
| 12 | QAILCWGELMTLATW | 31 | |
| 13 | WGELMTLATWVGSNL | 32 | CD8 (B/c); |
| | | | CD4 (B/c); |
| | | | CD8 (HLA) |
| 14 | TLATWVGSNLEDPAS | 33 | CD8 (B/c); |
| | | | CD4 (B/c) |
| 15 | VGSNLEDPASRELVV | 34 | CD8 (B/c); |
| | | | CD4 (B/c) |
| 16 | EDPASRELVVSYVNV | 35 | CD8 (B/c); |
| | | | CD4 (B/c) |
| 17 | RELVVSYVNVNMGLK | 36 | CD8 (B/c); |
| | | | CD4 (B/c) |
| 18 | SYVNVNMGLKIRQLL | 37 | |
| 19 | NMGLKIRQLLWFHIS | 38 | CD4 (B/c) |
| 20 | IRQLLWFHISCLTFG | 39 | CD4 (B/c) |
| 21 | WFHISCLTFGRETVI | 40 | CD4 (B/c) |
| 22 | CLTFGRETVIEYLVS | 41 | CD4 (B/c) |
| 23 | RETVIEYLVSFGVWI | 42 | CD4 (B/c) |
| 24 | EYLVSFGVWIRTPPA | 43 | |
| 25 | FGVWIRTPPAYRPPN | 44 | |
| 26 | RTPPAYRPPNAPILS | 45 | CD8 (Bl/6) |
| 27 | YRPPNAPILSTLPET | 46 | |
| 28 | APILSTLPETTVVRR | 47 | CD4 (Bl/6) |
| 29 | TLPETTVVRRRDRGR | 48 | CD8 (Bl/6) |
| 30 | TVVRRRDRGRSPRRR | 49 | |
| 31 | RDRGRSPRRRTPSPR | 50 | |
| 32 | SPRRRTPSPRRRRSQ | 51 | |
| 33 | TPSPRRRRSQSPRRR | 52 | |
| 34 | RRRSQSPRRRRRSQSR | 53 | |
| 35 | SPRRRRRSQSRESQC | 54 | |

B/c = BALB/c; Bl/6 = C57Bl/6; HLA = HLA-A2

TABLE 4
Epitope-Optimized PoIN Peptides

| | Amino Acid | SEQ ID | |
|---|---|---|---|
| Peptide | Sequence | NO: | Prime |
| 1 | PLSYQHFRKLLLLDE | 55 | |
| 2 | HFRKLLLLDEEAGPL | 56 | |
| 3 | LLLDEEAGPLEEELP | 57 | |
| 4 | EAGPLEEELPRLADE | 58 | |
| 5 | EEELPRLADEGLNRR | 59 | |
| 6 | RLADEGLNRRVAEDL | 60 | |
| 7 | GLNRRVAEDLNLGNL | 61 | |
| 8 | VAEDLNLGNLNVSIP | 62 | |
| 9 | NLGNLNVSIPWTHKV | 63 | |
| 10 | NVSIPWTHKVGNFTG | 64 | CD4 (B/c) |
| 11 | WTHKVGNFTGLYSST | 65 | |
| 12 | GNFTGLYSSTVPVFN | 66 | |
| 13 | LYS STVPVFNPEWQT | 67 | |
| 14 | VPVFNPEWQTPSFPK | 68 | CD4 (B/c) |
| 15 | PEWQTPSFPKIHKLQE | 69 | |
| 16 | PSFPKIHKLQEDIVDR | 70 | |
| 17 | IHKLQEDIVDRCKQFV | 71 | |
| 18 | EDIVDRCKQFVGPLTV | 72 | |
| 19 | RCKQFVGPLTVNEKRR | 73 | |
| 20 | VGPLTVNEKRRLKLIM | 74 | |
| 21 | VNEKRRLKLIMPARFY | 75 | |
| 22 | RLKLIMPARFYPNVTK | 76 | |
| 23 | MPARFYPNVTKYLPLD | 77 | |
| 24 | YPNVTKYLPLDKGIKP | 78 | |
| 25 | KYLPLDKGIKPYYPEH | 79 | |
| 26 | DKGIKPYYPEHAVNHY | 80 | |
| 27 | PYYPEHAVNHYFQTRH | 81 | |
| 28 | HAVNHYFQTRHYLHTL | 82 | |
| 29 | YFQTRHYLHTLWKAGI | 83 | |
| 30 | HYLHTLWKAGILYKRE | 84 | |
| 31 | LWKAGILYKRETTRSA | 85 | |
| 32 | ILYKRETTRSASFCGS | 86 | |
| 33 | ETTRSASFCGSPYSWE | 87 | |
| 34 | ASFCGSPYSWEQELQH | 88 | CD8 (Bl/6); |
| | | | CD8 (B/c); |
| | | | CD8 (HLA) |
| 35 | SPYSWEQELQHGSCWW | 89 | CD8 (Bl/6); |
| | | | CD8 (B/c); |
| | | | CD8 (HLA) |
| 36 | EQELQHGSCWWLQFRN | 90 | |
| 37 | HGSCWWLQFRNSKPCS | 91 | CD8 (Bl/6); |
| | | | CD8 (B/c); |
| | | | CD8 (HLA) |
| 38 | WLQFRNSKPCSEYCLT | 92 | CD8 (Bl/6); |
| | | | CD8 (B/c); |
| | | | CD8 (HLA) |
| 39 | NSKPCSEYCLTHLVNL | 93 | CD8 (Bl/6) |
| 40 | SEYCLTHLVNLLEDWG | 94 | CD8 (Bl/6); |
| | | | CD8 (B/c); |
| | | | CD8 (HLA) |
| 41 | THLVNLLEDWGPCDEH | 95 | |
| 42 | LLEDWGPCDEHGEHHI | 96 | |
| 43 | GPCDEHGEHHIRIPRT | 97 | |
| 44 | HGEHHIRIPRTPARVT | 98 | |
| 45 | IRIPRTPARVTGGVFL | 99 | |
| 46 | TPARVTGGVFLVDKNP | 100 | |
| 47 | TGGVFLVDKNPHNTAE | 101 | |
| 48 | LVDKNPHNTAESRLVV | 102 | |
| 49 | PHNTAESRLVVDFSQF | 103 | |
| 50 | ESRLVVDFSQFSRGIT | 104 | CD8 (Bl/6); |
| | | | CD8 (B/c); |
| | | | CD8 (HLA) |
| 51 | VDFSQFSRGITRVSWP | 105 | CD8 (Bl/6); |
| | | | CD8 (B/c); |
| | | | CD8 (HLA) |
| 52 | FSRGITRVSWPKFAVP | 106 | |
| 53 | TRVSWPKFAVPNLQSL | 107 | CD8 (Bl/6); |
| | | | CD8 (B/c); |
| | | | CD8 HLA) |
| 54 | PKFAVPNLQSLTNLLS | 108 | CD8 (Bl/6); |
| | | | CD8 (B/c); |
| | | | CD8 (HLA) |
| 55 | PNLQSLTNLLSSNLSW | 109 | CD8 (Bl/6) |
| 56 | LTNLLSSNLSWLSLDV | 110 | CD8 (Bl/6); |
| | | | CD8 (B/c); |
| | | | CD8 (HLA) |
| 57 | SSNLSWLSLDVSAAFY | 111 | |
| 58 | WLSLDVSAAFYHIPLH | 112 | CD8 (Bl/6); |
| | | | CD8 (B/c); |
| | | | CD8 (HLA) |
| 59 | VSAAFYHIPLHPAAMP | 113 | CD8 (Bl/6); |
| | | | CD8 (B/c); |
| | | | CD8 (HLA) |

B/c = BALB/c; Bl/6 = C57Bl/6; HLA = HLA-A2

TABLE 5
Epitope-Optimized PoIC Peptides

| | Amino Acid | SEQ | |
|---|---|---|---|
| Peptide | Sequence | ID NO: | Prime |
| 1 | HLLVGSSGLSRYVAR | 114 | |
| 2 | SSGLSRYVARLSSNSR | 115 | |
| 3 | RYVARLSSNSRIINHQ | 116 | |
| 4 | LSSNSRIINHQHGTMQ | 117 | |
| 5 | RIINHQHGTMQNLHDS | 118 | |
| 6 | QHGTMQNLHDSCSRNL | 119 | |
| 7 | QNLHDSCSRNLYVSLL | 120 | |
| 8 | SCSRNLYVSLLLLYKT | 121 | |
| 9 | LYVSLLLLYKTFGRKL | 122 | |
| 10 | LLLYKTFGRKLHLYSH | 123 | CD8 (HLA) |
| 11 | TFGRKLHLYSHPIILK | 124 | |
| 12 | LHLYSHPIILKTKRWG | 125 | CD8 (B/c); |
| | | | CD8 (HLA) |
| 13 | HPIILKTKRWGYSLNF | 126 | CD8 (HLA) |
| 14 | KTKRWGYSLNFMGYVI | 127 | |
| 15 | GYSLNFMGYVIGSWGS | 128 | CD8 (HLA) |
| 16 | FMGYVIGSWGSLPQDH | 129 | |
| 17 | IGSWGSLPQDHIIQKI | 130 | |
| 18 | SLPQDHIIQKIKECFR | 131 | |
| 19 | HIIQKIKECFRKLPVN | 132 | |
| 20 | IKECFRKLPVNRPIDW | 133 | |
| 21 | RKLPVNRPIDWKVCQR | 134 | |
| 22 | NRPIDWKVCQRIVGLL | 135 | |
| 23 | WKVCQRIVGLLGFAAP | 136 | |
| 24 | RIVGLLGFAAPFTQCG | 137 | |
| 25 | LGFAAPFTQCGYPALM | 138 | |
| 26 | PFTQCGYPALMPLYAC | 139 | CD8 (HLA) |
| 27 | GYPALMPLYACIQSKQ | 140 | |
| 28 | MPLYACIQSKQAFTFS | 141 | CD8 (B/c); |
| | | | CD8 (HLA) |
| 29 | CIQSKQAFTFSPTYKA | 142 | CD8 (HLA) |
| 30 | QAFTFSPTYKAFLSKQ | 143 | |
| 31 | SPTYKAFLSKQYLNLY | 144 | CD8 (Bl/6); |
| | | | CD8 (HLA) |
| 32 | AFLSKQYLNLYPVARQ | 145 | CD8 (Bl/6) |
| 33 | QYLNLYPVARQRPGLC | 146 | |
| 34 | YPVARQRPGLCQVFAD | 147 | CD8 (HLA) |
| 35 | QRPGLCQVFADATPTG | 148 | CD4 (B/c) |
| 36 | CQVFADATPTGWGLAM | 149 | CD8 (B/c); |
| | | | CD8 (HLA) |
| 37 | DATPTGWGLAMGHQRM | 150 | CD8 (HLA) |
| 38 | GWGLAMGHQRMRGTFV | 151 | |
| 39 | MGHQRMRGTFVAPLPI | 152 | CD4 (B/c); |
| | | | CD8 (HLA) |
| 40 | MRGTFVAPLPIHTAEL | 153 | |
| 41 | VAPLPIHTAELLAACF | 154 | |
| 42 | IHTAELLAACFARSRS | 155 | CD8 (HLA) |
| 43 | LLAACFARSRSGAKIL | 156 | |
| 44 | FARSRSGAKILGTDNS | 157 | CD8 (B/c); |
| | | | CD8 (HLA) |
| 45 | SGAKILGTDNSVVLSR | 158 | CD8 (HLA) |
| 46 | LGTDNSVVLSRKYTSF | 159 | |
| 47 | SVVLSRKYTSFPWLLG | 160 | |
| 48 | RKYTSFPWLLGCAANW | 161 | CD8 (HLA) |
| 49 | FPWLLGCAANWILRGT | 162 | |
| 50 | GCAANWILRGTSFVYV | 163 | |
| 51 | WILRGTSFVYVPSALN | 164 | CD4 (B/c) |
| 52 | TSFVYVPSALNPADDP | 165 | |
| 53 | VPSALNPADDPSRGRL | 166 | |
| 54 | NPADDPSRGRLGLSRP | 167 | |
| 55 | PSRGRLGLSRPLLRLP | 168 | CD4 (B/c) |
| 56 | LGLSRPLLRLPFRPTT | 169 | |
| 57 | PLLRLPFRPTTGRTSL | 170 | |
| 58 | PFRPTTGRTSLYAVSP | 171 | |
| 59 | TGRTSLYAVSPSV | 172 | |

B/c = BALB/c; Bl/6 = C57Bl/6; HLA = HLA-A2

TABLE 6
Epitope-Optimized Core Pool

| Core Matrix | A | B | C | D | E | F |
|---|---|---|---|---|---|---|
| G | 1 | 2 | 3 | 4 | 5 | 6 |
| H | 7 | 8 | 9 | 10 | 11 | 12 |
| I | 13 | 14 | 15 | 16 | 17 | 18 |
| J | 19 | 20 | 21 | 22 | 23 | 24 |
| K | 25 | 26 | 27 | 28 | 29 | 30 |
| L | 31 | 32 | 33 | 34 | 35 | |

TABLE 7
Epitope-Optimized PolN Pool

| Pol N Matrix | A | B | C | D | E | F | G | H |
|---|---|---|---|---|---|---|---|---|
| I | 1 | 2 | 3 | 4 | 5 | 6 | 7 | 8 |
| J | 9 | 10 | 11 | 12 | 13 | 14 | 15 | 16 |
| K | 17 | 18 | 19 | 20 | 21 | 22 | 23 | 24 |
| L | 25 | 26 | 27 | 28 | 29 | 30 | 31 | 32 |
| M | 33 | 34 | 35 | 36 | 37 | 38 | 39 | 40 |
| N | 41 | 42 | 43 | 44 | 45 | 46 | 47 | 48 |
| O | 49 | 50 | 51 | 52 | 53 | 54 | 55 | 56 |
| P | 57 | 58 | 59 | | | | | |

TABLE 8
Epitope-Optimized PolC Pool

| Pol C Matrix | A | B | C | D | E | F | G | H |
|---|---|---|---|---|---|---|---|---|
| I | 1 | 2 | 3 | 4 | 5 | 6 | 7 | 8 |
| J | 9 | 10 | 11 | 12 | 13 | 14 | 15 | 16 |
| K | 17 | 18 | 19 | 20 | 21 | 22 | 23 | 24 |
| L | 25 | 26 | 27 | 28 | 29 | 30 | 31 | 32 |
| M | 33 | 34 | 35 | 36 | 37 | 38 | 39 | 40 |
| N | 41 | 42 | 43 | 44 | 45 | 46 | 47 | 48 |
| O | 49 | 50 | 51 | 52 | 53 | 54 | 55 | 56 |
| P | 57 | 58 | | | | | | |

After the boost, which was tested in C57B1/6 , BALB/c and HLA-A2 tg mice, increases in responses were mainly seen for inserts and at vector doses that upon priming induced suboptimal responses, i.e., for Core tested at the 1×10⁹ vp vector dose (FIG. 4A-4C). Although booster immunization failed to increase the response to PolN or PolC when vectors were injected at high doses, the boost nevertheless broadened the T cell responses (FIG. 5A-5C)

### Immunogenicity Summary

The above results illustrate that:
The vaccines are immunogenic: PolN>PolC>Core for CD8+ T cells; Core>PolC>PolN for CD4+ T cell responses;
Immune responses can be boosted by a heterologous vaccine carrier;
Immune responses are broad; and
The breadth of the T cell responses increases after the boost.

### Effect of Vaccination on HBV Titers Low Dose AAV-1.3HBV Challenge

A group of 3 mice were challenged with 1×10¹⁰, 1×10¹¹ or 1.5×10¹¹ vg of AAV-1.3HBV and were vaccinated with AdC6-gDPolN 8 weeks later. Viral titers were tested 8 weeks after vaccination and compared to pre-vaccination titers. FIG. 6 shows viral changes from baseline for each treatment group.

### Epitope Shifting

CD8+ T cells to HBV antigens become exhausted during chronic HBV infections. Progression towards exhaustion is more rapid and pronounced for CD8+ T cells to dominant, as compared to subdominant, epitopes. The underlying reason is that exhaustion is driven by overwhelming antigen-driven stimulation through the T cell receptor; dominant epitopes are presented at higher levels on MHC class I antigens expressed by antigen presenting cells than subdominant epitopes with lower avidity to their restricting elements. Typical vaccine approaches primarily induce immune responses to dominant epitopes. Therapeutic vaccines should take into account loss of T cells to dominant epitopes during chronic virus infections and should be designed to favor expansion of CD8+ T cells to subdominant epitopes, which have a higher likelihood of resisting disease-driven exhaustion, translating to superior disease control.

The epitope profile in naïve mice immunized with an adenovirus vector comprising a nucleic acid sequence encoding the HBV polymerase N-terminal domain (PolN) fused to the herpes simplex virus glycoprotein D (“AdC6-gDPolN”, wherein the amino acid sequence of gDPolN is SEQ ID NO: 16) was determined. Responses in mice that had not been pre-treated with the AAV8-1.3HBV vector were compared to those obtained in mice infected with an AAV8 vector expressing the 1.3HBV genome prior to vaccination with the AdC6-gDPolN. The AAV8-1.3HBV vector induced high titers of HBV in serum, which could drive CD8+ T cell exhaustion.

In the first series of experiments a peptide pool matrix was used to identify epitopes in mice vaccinated with the AdC6-gDPolN vector, but not challenged with an AAV-1.3HBV vector. A number of regions in these naïve mice were identified that elicited potent responses (e.g. greater than 1% IFN-γ production CD8+CD44+ T cells. FIG. 7A and FIG. 8A. In the second experiment, mice were challenged with 1×10¹⁰ virus genomes (vg) of the AAV-1.3HBV vector, were vaccinated 4 weeks later with an AdC6 vector expressing the same HBV polymerase sequence (gDPolN) as in the initial experiment in non-challenged mice, and ten weeks thereafter the HBV PolN-specific CD8+ T cell epitope profile was determined using peptide pool matrices on splenocytes from the mice that had been challenged prior to vaccination. FIG. 7B and FIG. 8B. The experiment was repeated using more stringent conditions by challenging mice with a 1.5×10¹¹ vg dose of the AAV8-1.3HBV vector. Mice were again vaccinated 4 weeks later and were tested approximately 10 weeks after vaccination for CD8+ T cell responses to the peptide pool matrices. FIG. 7C and FIG. 8C. In both experiments, compared to the results obtained from unvaccinated mice, a shift was observed in the epitope profile in AAV8-1.3HBV infected mice, which at the time of vaccination had high viral loads between 10⁷-10⁹ vg per ml of serum. The effect was more pronounced in mice that had been challenged with a high dose of the AAV8-1.3HBV vector. In both experiments a reduction in responses were observed. Furthermore, especially in mice challenged with the high dose of AAV8-1.3HBV, the results showed a loss of CD8+ T cells to many of the epitopes that showed immunodominance in uninfected vaccinated mice (e.g. within region represented by peptides 50 to 59, FIG. 8), a better preservation of epitopes that were subdominant (such as those within the region represented by peptides 2 to 8) as well as new epitopes, such as in the region presented by peptides 10 to 29. These data confirm a shift from recognition of dominant to recognition of subdominant epitopes.

Based on these data a new HBV polymerase N-
terminal domain insert (HBV PolN v2) was generated
(SEQ ID NO: 173):
HFRKLLLLDEEAGPLEEELPRLADEGLNRRVAEDLNLGNLPEWQTPSFPK
IHLQEDIVDRCKQFVGPLIVNEKRRLKLIMPARFYPNVIKYLPLDKGIKP
YYPEHAVNHYFQTRHYLHTLWKAGILYKRETTRSASFCGSPYSWEQELQH
GSCWWLQFRNSKPCSEYCLTHLVNLLEDWGPCDEHGEHHIRIPRTPARVT

This insert induced CD8+ T cell responses mainly to subdominant epitopes to which responses remain intact in mice with high HBV viral loads.

Immunogenicity and Efficacy of gDCore, gDPo1N, and gDPo1C Vaccines

The immunogenicity and efficacy the AdC6-gDCore, AdC6-gDPolN, AdC6-gDPolC, AdC7-gDCore, AdC7-gDPolN, and AdC7-gDPolC vaccines in an AAV8-HBV mouse model were analyzed.

### Methods—Immunogenicity

C57B1/6 mice (n=5 per group) were injected with various doses of: AdC6-gDCore (gDCore nucleic acid sequence corresponding to SEQ ID NO: 15); AdC6-gDPolN (gDPolN nucleic acid sequence corresponding to SEQ ID NO: 17); or AdC6-gDPolC (gDPolC nucleic acid sequence corresponding to SEQ ID NO: 19). Two months after the first injection, AdC6 vector-immunized mice were boosted with AdC7 vectors containing the same insert (e.g. AdC7-gDCore, AdC7-gDPolN, or AdC7-gDPolC). Mice were bled at 14 days and 56 days after the injection and T cell frequencies to the various HBV inserts were analyzed by intracellular cytokine staining (ICS) for interferon (IFN)-γ upon stimulation of cells with overlapping peptides representing the HBV sequences. Control cells were cultured without peptides. Frequencies and phenotype of CD8+ T cells to one immunodominant epitope within PolN were tested for by staining with an MHC I tetramer. The breadth and specificity of CD8+ T cell responses to individual peptides within a target sequence was performed via epitope mapping of splenocytes (CD8+ T cells tested by ICS for IFN-γ).

To assess CD8+ T cells in the liver, C57B1/6 mice (n=8 per group) received intravenous administration of 1×10¹⁰ viral genomes (vg) of AAV8-1.3HBV, 1×10¹¹ vg of AAV8-1.3HBV, or nothing via their tail vein, and 4 weeks later received a single IM injection of 5×10⁹ viral particles (vp) of AdC6-gDPolN. Eight weeks after the IM injection, mice were sacrificed, livers were removed, and lymphocytes were isolated and stained with T cell markers and a tetramer recognizing the T cell receptor to an immunodominant epitope present in the PolN sequence.

In a separate experiment, three groups of C57B1/6 mice (n=4 per group) received a single IM injection of 5×10⁹ vp of AdC6-gDPolN at four weeks (-) or received either intravenous administration of 1×10¹¹ viral genomes (vg) of AAV8-1.3HBV via their tail vein with or without a single IM injection of 5×10⁹ vp of AdC6-gDPolN four weeks later. Approximately 2 months after administration of AAV8-1.3HBV, mice were sacrificed, livers were removed and liver slices were prepared from each of the three groups, stained with hematoxylin and eosin and evaluated for lymphocytic infiltrates. From the same experiment, cells were stained with a specific tetramer and fluorochrome labeled antibodies to T-bet (clone 4B10, BV785 stain) or antibodies to PD-1 (clone 29F.1Al2, BF605 stain), TIM-3 (clone RMT3-23, Pe/Cy7 stain), CTLA-4 (clone UC10-4B9, PE stain), or LAG-3 (clone C9B7W, BV650 stain). Cells were analyzed by flow cytometry and gated on CD44+CD8 tetramer positive cells, which were then gated on the markers. Percent marker positive cells were identified from histograms in comparison to naïve T cells.

### Methods—Efficacy

AAV8-1.3HBV Vector Studies—To assess the impact of AdC6-gDPolN on chronic HBV virus exposure, C57B1/6 mice (n=8 per group) were challenged intravenously via their tail vein with 1×10¹⁰ vg of AAV8-1.3HBV and four weeks later immunized with a single IM injection of 5×10⁹ vp of AdC6-gDPolN. HBV DNA viral titers were evaluated by qPCR; pre- and post-vaccination changes from baseline (log₁₀ copies/mL) were reported. Viral genome copy numbers were assessed at four, six, eight, ten, and twelve weeks after AAV8 challenge. Viral dynamics were assessed by PCR over time and the change in log10 in HBV copies per mL were assessed. The number of mice showing a one, two or three log reductions at different points after treatment was assessed.

Impact of chronic HBV virus exposure on CD8+ T cell antigen recognition over time—The effect of AAV8-1.3HBV on vaccine-induced hepatic CD8+ T cells was assessed. The epitope profile in splenocytes of naïve mice immunized with a single IM injection of 5×10⁹ vp of AdC6-gDPolN was determined 4 weeks after vaccination. Mice challenged with 1×10¹⁰ and 1.5×10¹¹ vg of AAV8-1.3HBV and subsequently vaccinated with 5×10⁹ vp of AdC6-gDPolN 4 weeks later had CD8+ T cell epitope profiles in splenocytes performed 10 weeks after vaccination (14 weeks after AAV injection). Epitope profiles between AAV-naïve and AAV-treated vaccinated animals were compared. PolN-specific CD8+ T cells from liver were analyzed for differentiation markers.

### Results

Immunogenicity—Vaccination induced robust and sustained CD8+ T cell responses to PolN (median frequencies over all circulating CD8+ T cells: 6.0%) and lower responses to PolC and core (median frequencies: 1.0% & 0.4%, respectively; FIG. 9A and FIG. 9B). Boosting at 8 weeks increased responses to all regions with significant changes being observed for core (p=0.007) (FIG. 9C). FIGS. 9A-9C show % CD8+ T cells over all CD8+ T cells for individual mice with medians indicated by the lines. Vaccination induced broad epitope recognition by CD8+ T cells that was further enhanced after boosting (27% to 34%; FIG. 10).

At week 12 following AdC6-gDPolN vaccination, AAV8-1.3HBV-infected vaccinated mice showed a preferential increase in hepatic CD8+ infiltrates (FIG. 11A-11B and FIG. 12A-12F), a decreased presence of vaccine-induced HBV-specific CD8+ T cells (FIG. 11A and FIG. 11B) and slightly reduced levels of T-bet (suggestive of loss of effector functions) (FIG. 13A-13B). FIG. 11A shows the % CD8+ T cells over all recovered lymphocytes from individual livers. FIG. 11B shows percent tetramer positive CD8⁺ cells, which were identified from histograms in comparison to naïve T cells. No clear pattern of cellular markers suggestive of T cell differentiation to an exhaustion phenotype was observed, however, between vaccinated AAV1.3HBV-infected and -uninfected mice (FIG. 13A-13B).

Efficacy—Following a single IM injection of the AdC6-gDPolN vector, AAV8-1.3HBV-infected mice had multi-log HBV DNA declines in serum that persisted throughout the 8-week post vaccination period (FIG. 14). Post vaccination, median declines in serum HBV DNA viral load levels at four and eight weeks were 0.86 and 2.69 log₁₀ cps/mL, respectively (FIG. 14A). At week 8, all animals had a >1 log₁₀ cps/mL, 6/7 (86%) had >2 log₁₀ cps/mL, and 2/7 (29%) had >3 log₁₀ cps/mL declines from baseline (FIG. 14B).

Following a single AdC6-gDPolN vector injection, distinct CD8+ T cell recognition patterns to PolN peptides in splenocytes were observed when AAV-HBV-infected and naïve mice were compared. FIG. 15A and FIG. 15B illustrate the results from experiments in which mice were first injected with AAV-1.3HBV and then four weeks later boosted with 10¹⁰ vp of the AdC6-gDPolN vector, splenocytes were harvested 8 weeks after the immunization and tested by ICS for IFN-γ upon a short in vitro stimulation with individual peptides spanning the sequence of PolN. Background frequencies obtained without peptide were subtracted. FIG. 15A shows the peptide recognition profile of mice that received the AdC6-gDPolN vaccine only followed by those that were first injected with the indicated doses of the AAV8-1.3HBV vector. The pie graphs in FIG. 15B show the corresponding responses to peptides that reached the threshold of 0.1% of all CD44⁺ CD8⁺ cells (data correspond to those in FIG. 15A). Each slice/color represents the frequency of the response to an individual peptide with size showing the proportion of the total; only responses greater than 0.1% were included. Pullouts indicate epitopes only recognized in AAV8-1.3HBV infected mice. It was found that pre-treatment with AAV reduced both the number of epitopes recognized after a single IM prime and the magnitude of the immune response as the sum total of IFN-γ producing CD8+ T cells over the pool of CD8⁺ T cells. AAV pre-treatment shifted the T cell recognition to new epitopes, which represent roughly a third of the detectable CD8⁺ T cell response. The percentage of functional HBV-specific CD8+ T cell responses were highest in naïve mice (4.4%, FIG. 15B) but decreased in the presence of low and high dose AAV8-1.3HBV (2.0% & 0.6%; respectively, FIG. 15B). AAV8-1.3HBV-uninfected animals showed strong CD8+ T cell responses to a number of epitopes, which were decreased and shifted in AAV-HBV-infected animals to include T cell recognition of new epitopes.

### Discussion

An HBV therapeutic vaccine that targets early CD8+ T cell activation using gD as a genetically encoded checkpoint inhibitor was generated and was shown to:
Induce potent and durable CD8+ T cell responses to key HBV antigens (FIG. 9);
Stimulate very broad CD8+ T cell responses (FIG. 10) that included sub-dominant epitope recognition (FIG. 15); and
Achieve sustained multi-log HBV DNA viral load reductions in an AAV mouse model (FIG. 14) with preferential trafficking of functional CD8+ T cells to the liver (FIGS. 11 and 12).

In the disclosed AAV studies, AAV-induced HBV infection caused loss of CD8+ T cell recognition to dominant epitopes of PolN following vaccination with AdC6-gDPolN (FIG. 15). Without intending to be bound by theory, it is believed that it is the breadth of the CD8+ T cells induced by gD and their ability to recognize subdominant epitopes that led to a sustained immune response and multi-log suppression of HBV.

### Immunogenicity of AdC6/7-gDPoIN in Blood and Liver Following Vaccination in AAV-Induced HBV-Infected Animals

The following studies were performed to evaluate CD8⁺ T cell responses to the AdC6-gDPolN vaccine in blood, spleens, and livers of animals in the presence of pre-existing AAV-induced HBV infection.

Experiment #1—CD8⁺ T cell responses in AAV8-1.3HBV Infected Mice: Response Kinetics in Blood

Purpose—To assess the effect of sustained titers of HBV antigen on CD8⁺ T cell responses to the gDPolN antigen as expressed within the AdC6 vector.

Methods—C57B1/6 mice were injected i.v. with the 10¹⁰ of the AAV8-1.3HBV vector. Four weeks later they were vaccinated with 5×10⁹ vp of the AdC6-gDPolN vector. Control mice received only the AdC6-gDPolN vector. Naïve mice served as additional controls. Mice were boosted 2 months later with the same dose of the AdC7-gDPolN vaccine. Blood was collected at various times after the prime and the boost and PBMCs were tested for IFN-γ-producing CD8⁺ T cells.

Results—As shown in FIG. 16, mice mounted a vigorous PolN-specific CD8⁺ T cell response 2 weeks after vaccination, which gradually declined by week 8 and then increased again after the boost. The CD8⁺ T cell response was more stable after the boost than after the prime. At most time points tested responses were lower in mice that had been injected with the AAV8-1.3HBV vector than in the controls that had not been injected with an AAV vector.

### Experiment #2—CD8⁺ T Cell Responses in AAV8-1.3HBV Infected Mice: Responses in Liver

Purpose—To assess CD8⁺ T cell responses including markers indicative of T cell exhaustion in livers of AAV8-1.3HBV-infected, vaccinated mice.

Methods—C57B1/6 mice were injected i.v. with the 10¹⁰ or 10¹¹ vg of the AAV8-1.3HBV vectors. Four weeks later they were vaccinated with 5×10⁹ vp of the AdC6-gDPolN vector. Control mice received only the AdC6-gDPolN vector. Naïve mice served as additional controls. Mice were boosted 2 months later with the same dose of the AdC7-gDPolN vaccine.

To obtain hepatic lymphocytes, livers were cut into small fragments and treated with 2 mg/ml Collagenase P, 1 mg/ml DNase I (all from Roche, Basel Switzerland) and 2% FBS (Tissue Culture Biologicals, Tulare, CA) in L15 under agitation for 1 hour. Liver fragments were homogenized, filtrated through 70 μm strainers and lymphocytes were purified by Percoll-gradient centrifugation and washed with DMEM supplemented with 10% FBS. Lymphocytes were stained with a violet live/dead dye (Thermo Fisher Scientific), anti-CD8-APC (clone 53-6.7, BioLegend), anti-CD44-Alexa Flour 700 (clone IM7, BioLegend), anti-EOMES-Alexa Fluor 488 (clone Danl lmag, eBioscience), anti-PD1-BV605 (clone 29F.1Al2, BioLegend), anti-LAG3-BV650 (clone C9B7W, BioLegend), anti-T-bet-BV786 (clone 4B10, BioLegend), anti-CTLA-4-PE-A (clone UC10-4B9, BioLegend), anti-TIM-3-Pe-Cy7-A (clone RMT3-23, BioLegend), and an APC-labeled MHC class I tetramer (NIH tetramer Facility, Emory University, Atlanta GA) corresponding to amino acids 396-404 FAVPNLQSL (SEQ ID NO: 188) (peptide 55) of the HBV polymerase at +4° C. for 30 min in the dark. Cells were washed and were analyzed by a BD FACS Celesta (BD Biosciences, San Jose, CA) and DiVa software. Post-acquisition analyses were performed with FlowJo (TreeStar, Ashland, OR).

Results—The frequencies of CD8⁺ T cells within the lymphocytic liver infiltrates were analyzed. Frequencies of CD8⁺ T cells within the lymphocytic liver infiltrates were increased in vaccinated mice as compared to naïve mice, and further increases were seen in mice that prior to vaccination had been injected with the AAV8-1.3HBV vector (FIG. 17A). Frequencies of PolN-specific CD8⁺ T cells identified by staining with a tetramer specific for an epitope present in the PolN insert were reduced in AAV-1.3HBV-injected mice (FIG. 17B).

The phenotypes of the infiltrating tetramer⁺CD8+ T cells in comparison to naïve (i.e., tetramer⁻CD44⁻CD8⁺ T cells) were assessed by determining the mean fluorescent intensity of a dye linked to a given antibody (FIG. 18A-FIG. 18F) and by assessing the percentages (FIG. 19A-FIG. 19F) of CD8⁺ T cells that were positive for the indicated markers.

T-bet which controls a number of CD8⁺ T cell functions, was reduced on hepatic CD8⁺ T cells from mice that had been injected with AAV8-1.3HBV prior to vaccination in comparison the vaccine only group. Exhaustion markers were not increased in AAV8-1.3HBV-pre-treated groups suggesting that the observed loss of PolN-specific CD8⁺ T cells in presence of HBV was unlikely to be caused by classical CD8⁺ T cell exhaustion (FIG. 18A-FIG. 18F and FIG. 19A-FIG. 19F).

### Experiment #3—Breadth of the PolN-Specific CD8+ T Cell Response in AAV8-1.3HBV Infected Mice

Purpose—To assess if the presence of HBV affects the breadth of the CD8⁺ T cell response to PolN expressed within gD by the AdC vaccines.

Methods—Mice were injected i.v. with the 10¹⁰ or 10¹¹ vg of the AAV8-1.3HBV vectors and were boosted 2 months later with the corresponding AdC7 vectors. Control mice received only the AdC6-gDPolN vector. Mice were euthanized 10 weeks later and the pooled splenocytes were tested against pools of peptides in the non-AAV infected animal study. Results are provided in FIG. 20A-FIG. 20C.

In a second experiment, mice were injected i.v. with the 10¹⁰ or 10¹¹ vg of the AAV8-1.3HBV vectors. Four weeks later they were vaccinated with 5×10¹⁰ vp of the AdC6-gDPolN vector. Control mice received only the AdC6-gDPolN vector. Naïve mice served as additional controls. Splenocytes were analyzed 6 weeks later for IFN-γ-producing CD8+ T cells in response to individual peptides spanning the PolN sequence. Results are provided in FIG. 20D-FIG. 20F.

Results—The presence of HBV, especially high titers of HBV such as after injection with the 10¹¹ vg dose of AAV8-HBV1.3, not only reduced overall CD8⁺ T cell responses to the PolN sequence as presented by the AdC6-gDPolN vaccine but also caused a shift in the epitope recognition profile.

### Experiment #4—Functions of Hepatic PolN-Specific CD8+ T Cells in AAV8-1.3HBV Infected Mice

Purpose—To evaluate if liver-infiltrating PolN-specific CD8⁺ T cells remain functional in AAV8-1.3HBV infected mice.

Methods—In the first experiment, C57BL/6 mice were injected i.v. with 3×10¹¹ vg of AAV8-1.3HBV. One group was vaccinated 8 weeks later with 5×10¹⁰ vp of AdC6-gDPolN vector. The other group was left unvaccinated. Mice were euthanized 4.5 months later and splenocytes were tested for frequencies of CD8⁺ T cells producing IFN-γ in response to the PolN peptide pool.

In the second experiment, mice were injected with graded concentrations of AAV8-1.3HBV (1×10¹⁰, 4×10¹⁰, or 1×10¹¹). All mice were vaccinated 4 weeks later with 5×10¹⁰ vp of the AdC6-gDPolN vector. The mice were boosted 2 months later with the same dose of the AdC7-gDPolN vector. Mice were euthanized 2 months later and lymphocytes were isolated from livers and tested for CD8⁺ T cells producing IFN-γ in response to the PolN peptide pool. Cells were also stained with an antibody to Tox, a transcription factor that increases in exhausted T cells.

Results—As shown in FIG. 21, vaccine-induced CD8⁺ T cells remained functional in mice that had been injected with the AAV8-1.3HBV vector.

### Experiment #5—Effect of Vaccination of AAV8-1.3HBV Infected Mice on Liver Histology

Purpose—To assess if AdC6/7-gDPolN vaccination of AAV.8-1.3HBV-vaccinated mice causes sustained liver damage.

Methods—Mice were injected with 10¹⁰ vg of the AAV8-1.3HPV given i.v. One month later they were vaccinated with 5×10⁹ vp of the AdC6-gDPolN vector. The mice were boosted 2 months later with the same dose of the AdC7-gDPolN vector given at the same dose. The mice were euthanized ˜2 months later. Liver sections were collected and fixed in 10% formaldehyde. Sections (˜3 um in thickness) were prepared and stained with Hematoxylin Eosin (H&E). They were reviewed under a light microscope at 20× magnification.

Results—One out of 33 sections from mice that had received both the AAV vector and the vaccine showed a small lymphocytic infiltrate that was at the margin of the liver section.

As shown in FIG. 21B, following a single gDPolN vaccination in HLA-A2-tg mice, frequencies of IFN-γ producing hepatic CD8⁺ T cells were reduced in mice receiving AAV as compared to those that had only been vaccinated.

### Conclusions

CD8⁺ T cell responses to PolN were reduced in AAV8-1.3HBV infected mice. Nevertheless, they remained detectable.
Exhaustion markers were not increased in AAV8-1.3HBV pre-treated animals suggesting that the observed loss of PolN-specific CD8⁺ T cells in the presence of HBV was unlikely to be caused by classical CD8⁺ T cell exhaustion.
AAV-induced HBV-infection caused a shift in the epitope recognition profile of CD8⁺ T cell responses to PolN.
Vaccine-induced CD8⁺ T cells remained functional in mice that had been previously infected with the AAV8-1.3HBV vector.
The vaccine used in a prime boost regimen did not cause overt liver damage in HBV positive mice.

### Generation of HBV PoIN-PoIC-Core Constructs

Two multi-antigen inserts (second generation PolN-Po1C-Core and third generation PolN-Po1C-Core) were generated. The sequences of these inserts are shown below:

2^{nd} generation HBV vaccine insert (“HBV2”)
(Pol N (italics)-Pol C (underlined)-Core)
(SEQ ID NO: 174)
YIPLDKGIKPYYPEHAVNHYFQTRHYLHTLWK
AGILYKRETTRSASFCGSPYSWEQELQHGSCW
WLQFRNSKPCSEYCITHLVNILEDWGPCDEHG
EHHIRIPRTPARVTGGVFLVDKNPHNTAESRL
VVDFSQFSRGITRVSWPKFAVPNIQSLTNLLS
SNLSWESLDVQAFTFSPTYKAFLSKQYLNLYP
VARQRPGLCQVFADATPTGWGLAMGHQRMRGT
FVAPLPIHTAELLAACFARSRSGAKILGTDNS
VVLSRKYTSFPWLLGCAANWILRGTSFVYVPS
ALNPADDVGSNLEDPASRELVVSYVNVNMGLK
IRQLLWFHISCLTFGRETVIEYLVSFGVWIRT
PPAYRPPNAPILSTLPETTVVRRRDRGR
3^{rd} generation HBV vaccine insert (“HBV3”)
(Pol N (italics)-Pol C (underlined)-Core)
(SEQ ID NO: 175)
HFRKLLLLDEEAGPLEEELPRLADEGLNRRVA
EDLNIGNLPEWQTPSFPKIHLQEDIVDRCKQF
VGPLTVNEKRRLKLIMPARFYPNVTKYLPLDK
GIKPYYPEHAVNHYFQTRHYLHTLWKAGILYK
RETTRSASFCGSPYSWEQELQHGSCWWLQFRN
SKPCSEYCLTHLVNLLEDWGPCDEHGEHHIRI
PRTPARVTQAFTFSPTYKAFLSKQYLNLYPVA
RQRPGLCQVFADATPTGWGLAMGHQRMRGTFV
APLPIHTAELLAACFARSRSGAKILGTDNSVV
LSRKYTSFPWLLGCAANWILRGTSFVYVPSAL
NPADDVGSNLEDPASRELVVSYVNVNMGLKIR
QLLWFHISCLTFGRETVIEYLVSFGVWIRTPP
AYRPPNAPILSTLPETTVVRRRDRGR

The second generation HBV (“HBV2”) insert includes immuno-dominant PolN epitopes identified from mice that had not been infected with the AAV8-1.3HBV vector prior to vaccination. Many of these epitopes were found to be lost in a mouse model of chronic HBV infection brought about by pre-administering an AAV8-1.3HBV vector (as defined in “Epitope Shifting” above). The third generation HBV (“HBV3”) insert selects for contiguous regions of PolN that were preferentially recognized by mice with high loads of HBV (see above). Regions of Core and PolC were selected for both constructs using the following general formula: regions with the highest immune responses on either prime (FIG. 3) or boost (FIG. 5) regions in C57B1/6, BALBc and HLA-A2 tg mice, and with the aim of selecting a large contiguous region instead of selecting unique epitopes and inserting spacer sequences between them.

Genetic Integrity and Stability of the 2^{nd} and 3^{rd} Generation HBV Inserts (HBV2 and HBV3)

Western Blot—Purified recombinant viral vector preparations (AdC6-gDHBV2, AdC6-gDHBV3, AdC7-gDHBV2, and AdC7-gDHBV3) were evaluated for their ability to elicit transgene-product expression in vitro. To that end, Western Blot assays were performed to assess the expression of gD protein in cell lysates following cell culture infection with the vector of interest. Adherent HEK293 cell monolayers were infected with known quantities of the purified vector and harvested at 48 hours post-infection, resuspended in lysis and extraction buffer containing protease inhibitors, and lysed by sonication. The total protein extracts were denatured by the use of dithiothreitol as a redox agent and submitted to electrophoresis in a 12% Bis-Tris polyacrylamide gel (PAGE). Subsequent to protein separation by SDS-PAGE, the samples were transferred onto an activated polyvinylidene difluoride membrane by wet electrophoretic transfer. The membrane was immunostained for the detection of gD protein using the primary antibody to gD diluted to 1:1000 in saline (clone PA1-30233, Invitrogen, Carlsbad, CA) for 1 h at room temperature. Membranes were washed with 1× TBS-T prior to incubating with HRP-conjugated goat anti-rabbit secondary IgG (ab6721, Abcam, Cambridge UK) for 1 h at room temperature. This was followed by the addition of a luminol-based chemiluminescent substrate. The stained membrane was exposed to an autoradiography film and signal emission was evaluated after processing by an automated film developer. Following documentation of the gD protein expression in infected HEK293 cell lysates, the membrane was stripped and re-probed for the presence of β-actin in the total protein extract samples. This staining step was employed to evaluate the consistency of the PAGE sample loading step and thus better support the semi-quantitative analysis of the in vitro stimulation of gD protein expression by the recombinant viral vector.

Stability—To ensure the genetic integrity of the viral construct, the genetic stability of each recombinant viral vector lot was assessed through sequential viral passages in adherent HEK293 cell cultures. The recombinant virus pool resulting from each transfection was cultured under standard growing conditions for a total of 12 passages. In the last passage, the virus pool was expanded and the crude harvest purified by cesium chloride gradient. Following vector purification, viral DNA was isolated using the QIAGEN DNeasy Blood & Tissue Kit and evaluated by restriction enzyme digest with Ase I and Bgl II, two restriction enzymes that cleave the DNA template in distinct construct-specific pre-defined banding patterns. After digestion, samples were submitted to electrophoresis in 1% agarose gel containing ethidium bromide to allow for the visualization of the digested bands, followed by documentation of results using a digital gel imaging system. Viral preparations that exhibited banding patterns identical to those of an early passage virus were considered to have maintained the original molecular clone structure and thus deemed stable at the end of 12 viral passages.

Results—The banding patterns of viral vector DNAs remained stable after 12 passages compared to that after 5 passages indicating the vector genomes were stable (data not shown).

Immunogenicity of the 2^{nd} and 3^{rd} Generation HBV Inserts (HBV2 and HBV3) as Expressed by AdC6 or AdC7 Vectors

Purpose—To assess CD8⁺ T cell responses to the HBV2 and HBV3 inserts expressed by AdC6 vectors or AdC7 vectors.

Methods—Groups of C57B1/6 mice were injected with 5×10⁹ or 5×10¹⁰ vp of AdC6-gDHBV2 or AdC6-gDHBV3 vector. Mice injected with the same doses of the AdC6-gDPolN vector served as positive controls; naïve mice served as negative controls. Mice were bled 14 days later and PBMCs were tested for frequencies of CD8⁺ T cells producing IFN-γ in response to peptide pools corresponding to the HBV inserts. Four weeks later (6 weeks after vaccination) mice were bled again and tested with the PolN-specific tetramer. AdC6-gDHBV3 immunized mice were excluded as this insert lacks the epitope that corresponds to the tetramer.

Groups of C57B1/6 mice were injected with 5×10⁹ or 5×10¹⁰ vp of AdC7-gDHBV2 or 5×10¹⁰ vp of AdC7-gDHBV3 vector. Naïve mice served as negative controls. Mice were bled 14 days later and PBMCs were tested for frequencies of CD8⁺ T cells producing IFN-γ in response to peptide pools corresponding to the HBV inserts.

### Immunogenicity of AdC7 Prime/AdC6 Boost

Mice were bled ˜4 weeks later and PBMCs were retested by ICS for CD8⁺ T cells producing IFN-γ and/or TNF-α in response to the peptides for the inserts. Mice were boosted two months after the prime with the same dose of the heterologous vector expressing the same insert. PBMCs were tested by ICS 2 weeks later and pre- and post-boost CD8⁺ and CD4⁺ T cell responses were compared. The AdC7-gDHBV2 vector induced robust frequencies of CD8⁺ T cells producing IFN-γ and/or TNF-α after the prime. Frequencies increased after the AdC6-gDHBV2 boost and this was especially pronounced after the low vector doses and for CD8⁺ T cells producing IFN-γ. The AdC7-gDHBV3 vector was poorly immunogenic but CD8+ T cell responses became positive after the AdC6-gDHBV3 boost. In the same token CD4+ T cell responses were marginal after the prime but increased after the boost. There was no marked difference in CD4 responses to the HBV2 or HBV3 insert.

### Conclusions

Both the AdC6-gDHBV2 and AdC7-gDHBV2 vectors were highly immunogenic (FIG. 22A, FIG. 22B, and FIG. 23) and responses increased after a boost with a heterologous AdC vector expressing the same insert (FIG. 24).
The AdC7-gDHBV2 and AdC7-gDHBV3 vectors displayed borderline immunogenicity consistent with their design as they lack the epitope that corresponds to the tetramer being used (FIG. 22A and FIG. 23).
Boosting AdC7-gDHBV2 with AdC6-gDHBV2 enhances CD8⁺ T cell responses.

### Comparison of HBV DNA Viral Titers in AdC6-gDPolN, AdC6-gDHBV2, AdC6-gDHBV3, or AdC6-HBV2 AAV-Infected Mice

### Methods

Five groups of C57B1/6 mice were challenged with 1×10⁹ vg of AAV8-1.3HBV and were vaccinated 4 weeks later with 1×10¹⁰ vp of either AdC6-gDPolN (n=10), AdC6-gDHBV2 (n=10), AdC6-gDHBV3 (n=10), or AdC6-HBV2 without gD (n=10); AAV-infected, non-vaccinated animals (“naive”) (n=10) and non-AAV-infected, non-vaccinated animals (n=2-5) served as controls. Viral titers were tested 4 weeks after AAV injection (before vaccination) and compared to levels 4 weeks after vaccination (week 8 after AAV injection).

### Results

At week 8, the median HBV viral titers increased by 0.98 log₁₀ cps/mL in naïve mice, remained unchanged in AdC6-HBV2 vaccinated mice, and declined by −0.04, −1.09 and −2.13 log₁₀ cps/mL in AdC6-gDHBV3, AdC6-gDPolN and AdC6-gDHBV2 vaccinated animals, respectively (FIG. 25A). The results for individual mice are shown in FIG. 25B — all AdC6-gDPolN and AdC6-gDHBV2 vaccinated animals had greater than 1 and 2 log₁₀ copies/mL declines, respectively; in contrast, none of the naïve, AdC6-HBV2, or AdC6-gDHBV3 vaccinated animals had a 1 log₁₀ copies/mL or greater decline at Week 8.

Immunogenicity Studies for gDHBV2 and gDHBV3

The induction of CD8⁺ T cell responses and their breadth to segments of HBV core and polymerase contained in either gDHBV2 or gDHBV3 following a single prime injection or prime followed by a boost vaccination with a heterologous vector containing the same insert were evaluated.

### Experiment 1

Purpose: Assess IFN-γ⁺ CD8⁺ T cell responses following prime and boost vaccinations with gD-HBV2 and gD-HBV3 expressed by heterologous chimpanzee adenoviral vectors (AdC6 and AdC7) in C57B1/6 mice.

Methods: Four groups of five C57B1/6 mice were immunized via intramuscular injection as follows: (a) 5×10¹⁰ vp AdC7-gDHBV2 followed two months later by 5×10¹⁰ vp AdC6-gDHBV2; (b) 5×10⁹ vp AdC7-gDHBV2 followed two months later by 5×10⁹ vp AdC6-gDHBV2; (c) 5×10¹⁰ vp AdC7-gDHBV3 followed two months later by 5×10¹⁰ vp AdC6-gDHBV3; or (d) no vaccine. Blood was assessed by ICS for IFN-γ⁺ CD8⁺ T cell responses 2 and 6 weeks after the prime, prior to the boost, and then 2 and 4 weeks after the boost.

Results: At all time points tested each vaccine construct was found to induce IFN-γ⁺ CD8⁺ T cells. FIG. 26 shows the percent of parental IFN-γ and/or TNF-α producing CD8⁺ T cells (FIG. 26A), CD44+CD8+ T cells (FIG. 26B), CD4+ T cells (FIG. 26C) or CD44+CD4+ T cells (FIG. 26D). Immune responses as assessed by ICS from PBMCs of individual mice are shown two and eight weeks after the prime, as well as two and four weeks after the boost as the mean.

### Experiment 2

Purpose: Compare IFN-γ⁺ CD8⁺ T cell responses following different doses of prime and boost vaccinations with gD-HBV2 and gD-HBV3 to that with gD-PolN using heterologous chimpanzee adenoviral vectors (AdC6 and AdC7) in C57B1/6 mice.

Methods: Groups of C57B1/6 mice (n=5 mice/group) were immunized as follows:

gDPolN Groups
(a) 5×10⁹ vp AdC6-gDPolN followed three months later by 5×10⁹ vp AdC7-gDPolN; and
(b) 5×10¹⁰ vp AdC6-gDPolN followed three months later by 5×10¹⁰ vp AdC7-gDPolN
   gDHBV2 Groups
(c) 5×10⁹ vp AdC6-gDHBV2 followed three months later by 5×10⁹ vp AdC7-gDHBV2 and;
(d) 5×10¹⁰ vp AdC6-gDHBV2 followed three months later by 5×10¹⁰ vp AdC7-gDHBV2 gDHBV3 Groups
(e) 5×10⁹ vp AdC6-gDHBV3 followed three months later by 5×10⁹ vp AdC7-gDHBV3 and;
(f) 5×10¹⁰ vp AdC6-gDHBV3 followed three months later by 5×10¹⁰ vp AdC7-gDHBV3

### No Treatment Served as Controls

For all treatment groups, immunogenicity CD8+ T cell responses was from blood assessed by ICS for IFN-γ⁺ at two and six weeks after the prime, prior to the boost, and then two and six weeks after the boost. Immunogenicity was also assessed by tetramer staining using an APC-labeled MHC class I tetramer (NIH tetramer Facility, Emory University, Atlanta GA) corresponding to amino acids 396-404 FAVPNLQSL (peptide 55) of the HBV polymerase at week four after the prime. HBV3 does not contain the FAVPNLQSL peptide.

Results: At all time points, each vaccine tested was found to induce IFN-γ⁺ CD8⁺ T cells. Results obtained with the gDHBV2 vaccine were similar to those obtained with the gDPolN vaccine; the gDHBV3 vaccine was less immunogenic. Upon tetramer staining, frequencies of the specific CD8⁺ T cells were comparable between the two vaccines; a number of activation markers tended to be more highly expressed on tetramer+CD8+ T cells from the gDHBV2-immunized groups. FIG. 27 shows CD8⁺ T cells at multiple time points: four weeks after prime (FIG. 27A); two weeks after the boost (FIG. 27B); and four weeks after the boost (FIG. 27C). The graph shows the overall frequencies of CD8⁺ T cells producing IFN-γ⁺ as assessed by ICS.

FIG. 28 shows cytokine-producing CD4+ T cells at multiple time points: four weeks after prime (FIG. 28A); two weeks after the boost (FIG. 28B); and four weeks after the boost (FIG. 28C) as assessed by ICS. The dashed line indicates the cut-off for positive responses, based on the results from the naïve mice.

FIG. 29 shows the results of tetramer staining gated on either CD8+ T cells (FIG. 29A) or CD44+CD8+ T cells (FIG. 29B) at four weeks after the prime.

FIG. 30 shows the phenotypes of the tetramer+CD8+ T cells shown as the mean fluorescent intensity of a dye linked to the indicated antibody: FIG. 30A anti-PD1 antibody conjugated to BV605; FIG. 30B anti-LAG3 antibody conjugated to BV650; FIG. 30C anti-TIM3 antibody conjugated to Pe-Cy7-A; FIG. 30D anti-CTLA4 antibody conjugated to PE-A; FIG. 30E anti-EOMES antibody conjugated to AF488; and FIG. 30F anti-T-bet antibody conjugated to BV786.

### Experiment 3

The breadth of responses were assessed from pooled splenocytes of vaccinated C57BL/6 mice which were tested by ICS against the individual peptides present in the HBV vaccine inserts.

Methods: Four groups of five C57B1/6 mice were immunized via intramuscular injection as follows: (a) 5×10¹⁰ vp AdC7-gDHBV2 followed two months later by 5×10¹⁰ vp AdC6-gDHBV2; (b) 5×10⁹ vp AdC7-gDHBV2 followed two months later by 5×10⁹ vp AdC6-gDHBV2; (c) 5×10¹⁰ vp AdC7-gDHBV3 followed two months later by 5×10¹⁰ vp AdC6-gDHBV3; or (3) no vaccine. Animals were sacrificed eight weeks after the boost and pooled splenocytes were assessed by ICS for IFN-γ⁺ CD8⁺ T cell responses to individual HBV2 or HBV3 peptides (cut-off for positive responses set at 0.1%).

Results: Independent of the dose, the prime boost regimen with the gDHBV2 vaccines induced responses to several epitopes within core and polymerase. FIG. 31 shows the CD8⁺ T cell responses after a prime vaccination of 5×10¹⁰ vp AdC7-gDHBV2 followed two months later by vaccination with 5×10¹⁰ vp AdC6-gDHBV2. Numbers on the X axis correspond to the SEQ ID NO as provided herein. FIG. 32 shows the CD8+ T cell responses after a prime vaccination with 5×10⁹ vp AdC7-gDHBV2 followed two months later by vaccination with 5×10⁹ vp AdC6-gDHBV2. Numbers on the X axis correspond to the SEQ ID NO as provided herein. FIG. 33 shows the immunogenicity after a prime vaccination with 5×10¹⁰ vp AdC7-gDHBV3 followed two months later by vaccination with 5×10¹⁰ vp AdC6-gDHBV3. Numbers on the X axis correspond to the SEQ ID NO as provided herein.

### Experiment 4

The breadth of responses were assessed from pooled splenocytes of vaccinated BALB/c mice which were tested by ICS against the individual peptides present in the HBV vaccine inserts.

Methods: Five groups of five BALB/c mice were immunized via intramuscular injection as follows: (a) 5×10¹⁰ vp AdC6-gDHBV2; (b) 5×10¹⁰ vp AdC6-gDHBV3; (c) 5×10¹⁰ vp AdC7-gDHBV2; (d) 5×10¹⁰ vp AdC7-gDHBV3; or (e) no vaccine. 12 weeks post vaccination animals were sacrificed, spleens were collected and pooled splenocytes were assessed by ICS for IFN-γ⁺ CD8+ T cell responses to individual HBV2 or HBV3 peptides (cut-off for positive responses set at 0.1%).

Results: At week 12, each vaccine construct was found to be immunogenic across multiple regions of the Core and Polymerase genes delivered by the vaccine. FIG. 34 shows the immunogenicity of the AdC6-gDHBV2 and AdC7-gDHBV2 vaccines corresponding to the SEQ ID NO (X axis) as provided herein. Core, PolC, and PolN regions in both HBV2 constructs were immunogenic. FIG. 35 shows the immunogenicity of the AdC6-gDHBV3 and AdC7-gDHBV3 vaccines corresponding to the SEQ ID NO (X axis) as provided herein. Core, PolC, and PolN regions in both HBV3 constructs were immunogenic.

### Experiment 5

Methods: Five groups of C57B1/6 mice were challenged with 1×10⁹ vg of AAV8-1.3HBV and were vaccinated 4 weeks later (“prime vaccination”) with 1×10¹⁰ vp of either AdC6-gDPolN (n=10), AdC6-gDHBV2 (n=10), AdC6-gDHBV3 (n=10), or AdC6-HBV2 without gD (n=10); AAV-infected, non-vaccinated animals (n=10) and non-AAV-infected, non-vaccinated animals (n=2-5) serve as controls. Mice will be bled at various times after the injection and frequencies of insert-specific CD8+ and CD4+ T cells will be determined by intracellular cytokine staining (ICS) for IFN-γ. PCR will be performed at 2 weeks, 6 weeks, and 8 weeks after the prime vaccination, and a T cell assay will be performed at 4 weeks after the prime vaccination.

At 8 weeks following the prime vaccination, mice will be boosted with AdC7 vectors containing the same antigenic insert used in the prime vaccination (“boost vaccination”) and blood and serum will be tested for CD8+/CD4+ T cell as previously described at different time points after vaccination. PCR will be performed at 2 weeks, 6 weeks, and 10 weeks after the boost vaccination, and a T cell assay will be performed at 4 weeks and 12 weeks after the boost vaccination.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in their entirety.

TABLE 9
Sequences

| | Sequence |
|---|---|
| Genotype A | MDIDPYKEFGATVELLSFLPSDFFPSVRDLLDTASAL |
| Consensus | YREALESPEHCSPHHTALRQAILCWGELMTLATWVGN |
| (SEQ ID NO: 1) | NLeDPASRDLVVNYVNTNMGLKIRQLLWFHISCLTFG |
| | RETVLEYLVSFGVWIRTPPAYRPPNAPILSTLPETTV |
| | VRRRDRGRSPRRRTPSPRRRRSQSPRRRRSQSRESQC |
| Genotype B | MDIDpYKEFGASvELLSFLPSDFFPSiRDLLDTAsAL |
| Consensus | YREALESPEHCSPHHTALRQAIlCWGELMNLATWVGS |
| (SEQ ID NO: 2) | NLeDPASRELVVsYVNVNMGLKiRQLLWFHISCLTFG |
| | RETVLEYLVSFGVWIRTPpAYRPpNAPILSTLPETTV |
| | VRRRGRSPRRRTPSPRRRRSQSPRRRRSQSREsQC |
| Genotype C | MDIDpYKEFGASVELLSFLPSDFFPSIRDLLDTASAL |
| Consensus | YREALESPEHCSPHHTALRQAILCWGELMNLATWVGS |
| (SEQ ID NO: 3) | NLEDPASRELVVsYVNVNMGLKiRQlLWFHISCLTFG |
| | RETVLEYLVSFGVWIRTPpAYRPPNAPILSTLPETTV |
| | VRRRGRSPRRRTPSPRRRRSQSPRRRSQSRESQC |
| Genotype D | MDIDPYKEFGAtVELLSFLPsDFFPSVRDLLDTASALYReAL |
| Consensus | ESPEHCSPHHTALRQAILCWGeLMtLATWVGgNLEDPaSRDL |
| (SEQ ID NO: 4) | VVSYVNTNmGLKFRQLLWFHISCLTFGReTViEYLVSFGVWI |
| | RTPpAYRPPNAPILSTLPETTVvRRRGRSPRRRTPSPRRRRS |
| | QSPRRRRSQSRESQC |
| Initial Core | MDIDPYKEFGAX₁VELLSFLPSDFFPSX₂DLLDTASALYREA |
| sequence | LESPEHCSPHHTALRQAILCWGELMX₃LATWVGX₄NLeDPAS |
| (SEQ ID NO: 5) | RX₅LVVX₆YVNX₇NMGLKX₈RQLLWFHISCLTFGRETVX₉EY |
| | LVSFGVWIRTPPAYRPPNAPILSTLPETTVVRRRX₁₀X₁₁GR |
| | SPRRRTPSPRRRRSQSPRRRRSQSRESQC |
| Epitope- | DIDPYKEFGATVELLSFLPSDFFPSIRDLLDTASALYREALE |
| optimized Core | SPEHCSPHHTALRQAILCWGELMTLATWVGSNLEDPASRELV |
| amino acid | VSYVNVNMGLKIRQLLWFHISCLTFGRETVIEYLVSFGVWIR |
| sequence | TPPAYRPPNAPILSTLPETTVVRRRDRGRSPRRRTPSPRRRR |
| (SEQ ID NO: 6) | SQSPRRRRSQSRESQC |
| Epitope- | GACATCGACCCCTACAAGGAGTTCGGCGCCACCGTGGAGCTG |
| optimized Core | CTGAGCTTCCTGCCCAGCGACTTCTTCCCCAGCATCAGGGAC |
| nucleotide | CTGCTGGACACCGCCAGCGCCCTGTACAGGGAGGCCCTGGAG |
| sequence | AGCCCCGAGCACTGCAGCCCCCACCACACCGCCCTGAGGCAG |
| (SEQ ID NO: 7) | GCCATCCTGTGCTGGGGCGAGCTGATGACCCTGGCCACCTGG |
| | GTGGGCAGCAACCTGGAGGACCCCGCCAGCAGGGAGCTGGTG |
| | GTGAGCTACGTGAACGTGAACATGGGCCTGAAGATCAGGCAG |
| | CTGCTGTGGTTCCACATCAGCTGCCTGACCTTCGGCAGGGAG |
| | ACCGTGATCGAGTACCTGGTGAGCTTCGGCGTGTGGATCAGG |
| | ACCCCCCCCGCCTACAGGCCCCCCAACGCCCCCATCCTGAGC |
| | ACCCTGCCCGAGACCACCGTGGTGAGGAGGAGGGACAGGGGC |
| | AGGAGCCCCAGGAGGAGGACCCCCAGCCCCAGGAGGAGGAGG |
| | AGCCAGAGCCCCAGGAGGAGGAGGAGCCAGAGCAGGGAGAGC |
| | CAGTGC |
| Epitope- | PLSYQHFRKLLLLDEEAGPLEEELPRLADEGLNRRVAEDLNL |
| optimized | GNLNVSIPWTHKVGNFTGLYSSTVPVFNPEWQTPSFPKIHLQ |
| polymerase N- | EDIVDRCKQFVGPLTVNEKRRLKLIMPARFYPNVTKYLPLDK |
| terminal amino | GIKPYYPEHAVNHYFQTRHYLHTLWKAGILYKRETTRSASFC |
| acid sequence | GSPYSWEQELQHGSCWWLQFRNSKPCSEYCLTHLVNLLEDWG |
| (SEQ ID NO: 8) | PCDEHGEHHIRIPRTPARVTGGVFLVDKNPHNTAESRLVVDF |
| | SQFSRGITRVSWPKFAVPNLQSLTNLLSSNLSWLSLDVSAAF |
| | YHIPLHPAAMP |
| Epitope- | CCCCTGAGCTACCAGCACTTCAGGAAGCTGCTGCTGCTGGAC |
| optimized | GAGGAGGCCGGCCCCCTGGAGGAGGAGCTGCCCAGGCTGGCC |
| polymerase N- | GACGAGGGCCTGAACAGGAGGGTGGCCGAGGACCTGAACCTG |
| terminus | GGCAACCTGAACGTGAGCATCCCCTGGACCCACAAGGTGGGC |
| nucleotide | AACTTCACCGGCCTGTACAGCAGCACCGTGCCCGTGTTCAAC |
| sequence | CCCGAGTGGCAGACCCCCAGCTTCCCCAAGATCCACCTGCAG |
| (SEQ ID NO: 9) | GAGGACATCGTGGACAGGTGCAAGCAGTTCGTGGGCCCCCTG |
| | ACCGTGAACGAGAAGAGGAGGCTGAAGCTGATCATGCCCGCC |
| | AGGTTCTACCCCAACGTGACCAAGTACCTGCCCCTGGACAAG |
| | GGCATCAAGCCCTACTACCCCGAGCACGCCGTGAACCACTAC |
| | TTCCAGACCAGGCACTACCTGCACACCCTGTGGAAGGCCGGC |
| | ATCCTGTACAAGAGGGAGACCACCAGGAGCGCCAGCTTCTGC |
| | GGCAGCCCCTACAGCTGGGAGCAGGAGCTGCAGCACGGCAGC |
| | TGCTGGTGGCTGCAGTTCAGGAACAGCAAGCCCTGCAGCGAG |
| | TACTGCCTGACCCACCTGGTGAACCTGCTGGAGGACTGGGGC |
| | CCCTGCGACGAGCACGGCGAGCACCACATCAGGATCCCCAGG |
| | ACCCCCGCCAGGGTGACCGGCGGCGTGTTCCTGGTGGACAAG |
| | AACCCCCACAACACCGCCGAGAGCAGGCTGGTGGTGGACTTC |
| | AGCCAGTTCAGCAGGGGCATCACCAGGGTGAGCTGGCCCAAG |
| | TTCGCCGTGCCCAACCTGCAGAGCCTGACCAACCTGCTGAGC |
| | AGCAACCTGAGCTGGCTGAGCCTGGACGTGAGCGCCGCCTTC |
| | TACCACATCCCCCTG CACCCCGCCGCCATGCCC |
| Epitope- | HLLVGSSGLSRYVARLSSNSRIINHQHGTMQNLHDSCSRNLY |
| optimized | VSLLLLYKTFGRKLHLYSHPIILKTKRWGYSLNFMGYVIGSW |
| polymerase C- | GSLPQDHIIQKIKECFRKLPVNRPIDWKVCQRIVGLLGFAAP |
| terminal amino | FTQCGYPALMPLYACIQSKQAFTFSPTYKAFLSKQYLNLYPV |
| acid sequence | ARQRPGLCQVFADATPTGWGLAMGHQRMRGTFVAPLPIHTAE |
| (SEQ ID NO: 10) | LLAACFARSRSGAKILGTDNSVVLSRKYTSFPWLLGCAANWI |
| | LRGTSFVYVPSALNPADDPSRGRLGLSRPLLRLPFRPTTGRT |
| | SLYAVSPSV |
| Epitope- | CACCTGCTGGTGGGCAGCAGCGGCCTGAGCAGGTACGTGGCC |
| optimized | AGGCTGAGCAGCAACAGCAGGATCATCAACCACCAGCACGGC |
| polymerase C- | ACCATGCAGAACCTGCACGACAGCTGCAGCAGGAACCTGTAC |
| terminal | GTGAGCCTGCTGCTGCTGTACAAGACCTTCGGCAGGAAGCTG |
| nucleotide | CACCTGTACAGCCACCCCATCATCCTGAAGACCAAGAGGTGG |
| sequence | GGCTACAGCCTGAACTTCATGGGCTACGTGATCGGCAGCTGG |
| (SEQ ID NO: 11) | GGCAGCCTGCCCCAGGACCACATCATCCAGAAGATCAAGGAG |
| | TGCTTCAGGAAGCTGCCCGTGAACAGGCCCATCGACTGGAAG |
| | GTGTGCCAGAGGATCGTGGGCCTGCTGGGCTTCGCCGCCCCC |
| | TTCACCCAGTGCGGCTACCCCGCCCTGATGCCCCTGTACGCC |
| | TGCATCCAGAGCAAGCAGGCCTTCACCTTCAGCCCCACCTAC |
| | AAGGCCTTCCTGAGCAAGCAGTACCTGAACCTGTACCCCGTG |
| | GCCAGGCAGAGGCCCGGCCTGTGCCAGGTGTTCGCCGACGCC |
| | ACCCCCACCGGCTGGGGCCTGGCCATGGGCCACCAGAGGATG |
| | AGGGGCACCTTCGTGGCCCCCCTGCCCATCCACACCGCCGAG |
| | CTGCTGGCCGCCTGCTTCGCCAGGAGCAGGAGCGGCGCCAAG |
| | ATCCTGGGCACCGACAACAGCGTGGTGCTGAGCAGGAAGTAC |
| | ACCAGCTTCCCCTGGCTGCTGGGCTGCGCCGCCAACTGGATC |
| | CTGAGGGGCACCAGCTTCGTGTACGTGCCCAGCGCCCTGAAC |
| | CCCGCCGACGACCCCAGCAGGGGCAGGCTGGGCCTGAGCAGG |
| | CCCCTGCTGAGGCTGCCCTTCAGGCCCACCACCGGCAGGACC |
| | AGCCTGTACGCCGTGAGCCCCAGCGTG |
| N-terminal HSV | MGGAAARLGAVILFVVIVGLHGVRGKYALADASLKMADPNRF |
| gD sequence | RGKDLPVLDQLTDPPGVRRVYHIQAGLPDPFQPPSLPITVYY |
| (SEQ ID NO: 12) | AVLERACRSVLLNAPSEAPQIVRGASEDVRKQPYNLTIAWFR |
| Signal peptide | MGGNCAIPITVMEYTECSYNKSLGACPIRTQPRWNYYDSFSA |
| in italics | VSEDNLGFLMHAPAFETAGTYLRLVKINDWTEITQFILEHRA |
| | KGSCKYALPLRIPPSACLSPQAYQQGVTVDSIGMLPRFIPEN |
| | QRTVAVYSLKIAGWHGP |
| C-terminal HSV | GPKAPYTSTLLPPELSETPNATQPELAPEDPEDSALLEDPVG |
| gD sequence | TVAPQIPPNWHIPSIQDAATPYHPPATPNNMGLIAGAVGGSL |
| (SEQ ID NO: 13) | LAALVICGIVYWMHRRTRKAPKRIRLPHIREDDQPSSHQPLF |
| | Y |
| gDCore amino | MGGAAARLGAVILFVVIVGLHGVRGKYALADASLKMADPNRF |
| acid sequence | RGKDLPVLDQLTDPPGVRRVYHIQAGLPDPFQPPSLPITVYY |
| Core underlined | AVLERACRSVLLNAPSEAPQIVRGASEDVRKQPYNLTIAWFR |
| (SEQ ID NO: 14) | MGGNCAIPITVMEYTECSYNKSLGACPIRTQPRWNYYDSFSA |
| Signal peptide | VSEDNLGFLMHAPAFETAGTYLRLVKINDWTEITQFILEHRA |
| in italics | KGSCKYALPLRIPPSACLSPQAYQQGVTVDSIGMLPRFIPEN |
| | QRTVAVYSLKIAGWHGPDIDPYKEFGATVELLSFLPSDFFPS |
| | IRDLLDTASALYREALESPEHCSPHHTALRQAILCWGELMTL |
| | ATWVGSNLEDPASRELVVSYVNVNMGLKIRQLLWFHISCLTF |
| | GRETVIEYLVSFGVWIRTPPAYRPPNAPILSTLPETTVVRRR |
| | DRGRSPRRRTPSPRRRRSQSPRRRRSQSRESQCGPKAPYTST |
| | LLPPELSETPNATQPELAPEDPEDSALLEDPVGTVAPQIPPN |
| | WHIPSIQDAATPYHPPATPNNMGLIAGAVGGSLLAALVICGI |
| | VYWMHRRTRKAPKRIRLPHIREDDQPSSHQPLFY |
| gDCore nucleic | atggggggggctgccgccaggttgggggccgtgattttgttt |
| acid sequence | gtcgtcatagtgggcctccatggggtccgcggcaaatatgcc |
| Core underlined | ttggcggatgcctctctcaagatggccgaccccaatcgcttt |
| (SEQ ID NO: 15) | cgcggcaaagaccttccggtcctggaccagctgaccgaccct |
| | ccgggggtccggcgcgtgtaccacatccaggcgggcctaccg |
| | gacccgttccagccccccagcctcccgatcacggtttactac |
| | gccgtgttggagcgcgcctgccgcagcgtgctcctaaacgca |
| | ccgtcggaggccccccagattgtccgcggggcctccgaagac |
| | gtccggaaacaaccctacaacctgaccatcgcttggtttcgg |
| | atgggaggcaactgtgctatccccatcacggtcatggagtac |
| | accgaatgctcctacaacaagtctctgggggcctgtcccatc |
| | cgaacgcagccccgctggaactactatgacagcttcagcgcc |
| | gtcagcgaggataacctggggttcctgatgcacgcccccgcg |
| | tttgagaccgccggcacgtacctgcggctcgtgaagataaac |
| | gactggacggagattacacagtttatcctggagcaccgagcc |
| | aagggctcctgtaagtacgccctcccgctgcgcatccccccg |
| | tcagcctgcctctccccccaggcctaccagcagggggtgacg |
| | gtggacagcatcgggatgctgccccgcttcatccccgagaac |
| | cagcgcaccgtcgccgtatacagcttgaagatcgccgggtgg |
| | cacgggcccgacatcgacccctacaaggagttcggcgccacc |
| | gtggagctgctgagcttcctgcccagcgacttcttccccagc |
| | atcagggacctgctggacaccgccagcgccctgtacagggag |
| | gccctggagagccccgagcactgcagcccccaccacaccgcc |
| | ctgaggcaggccatcctgtgctggggcgagctgatgaccctg |
| | gccacctgggtgggcagcaacctggaggaccccgccagcagg |
| | gagctggtggtgagctacgtgaacgtgaacatgggcctgaag |
| | atcaggcagctgctgtggttccacatcagctgcctgaccttc |
| | ggcagggagaccgtgatcgagtacctggtgagcttcggcgtg |
| | tggatcaggaccccccccgcctacaggccccccaacgccccc |
| | atcctgagcaccctgcccgagaccaccgtggtgaggaggagg |
| | gacaggggcaggagccccaggaggaggacccccagccccagg |
| | aggaggaggagccagagccccaggaggaggaggagccagagc |
| | agggagagccagtgcgggcccaaggccccatacacgagcacc |
| | ctgctgcccccggagctgtccgagacccccaacgccacgcag |
| | ccagaactcgccccggaagaccccgaggattcggccctcttg |
| | gaggaccccgtggggacggtggcgccgcaaatcccaccaaac |
| | tggcacatcccgtcgatccaggacgccgcgacgccttaccat |
| | cccccggccaccccgaacaacatgggcctgatcgccggcgcg |
| | gtgggcggcagtctcctggcagccctggtcatttgcggaatt |
| | gtgtactggatgcaccgccgcactcggaaagccccaaagcgc |
| | atacgcctcccccacatccgggaagacgaccagccgtcctcg |
| | caccagcccttgttttactag |
| gDPolN amino | MGGAAARLGAVILFVVIVGLHGVRGKYALADASLKMADPNRF |
| acid sequence | RGKDLPVLDQLTDPPGVRRVYHIQAGLPDPFQPPSLPITVYY |
| PolN underlined | AVLERACRSVLLNAPSEAPQIVRGASEDVRKQPYNLTIAWFR |
| (SEQ ID NO: 16) | MGGNCAIPITVMEYTECSYNKSLGACPIRTQPRWNYYDSFSA |
| Signal peptide | VSEDNLGFLMHAPAFETAGTYLRLVKINDWTEITQFILEHRA |
| in italics | KGSCKYALPLRIPPSACLSPQAYQQGVTVDSIGMLPRFIPEN |
| | QRTVAVYSLKIAGWHGPPLSYQHFRKLLLLDEEAGPLEEELP |
| | RLADEGLNRRVAEDLNLGNLNVSIPWTHKVGNFTGLYSSTVP |
| | VFNPEWQTPSFPKIHLQEDIVDRCKQFVGPLTVNEKRRLKLI |
| | MPARFYPNVTKYLPLDKGIKPYYPEHAVNHYFQTRHYLHTLW |
| | KAGILYKRETTRSASFCGSPYSWEQELQHGSCWWLQFRNSKP |
| | CSEYCLTHLVNLLEDWGPCDEHGEHHIRIPRTPARVTGGVFL |
| | VDKNPHNTAESRLVVDFSQFSRGITRVSWPKFAVPNLQSLTN |
| | LLSSNLSWLSLDVSAAFYHIPLHPAAMPGPKAPYTSTLLPPE |
| | LSETPNATQPELAPEDPEDSALLEDPVGTVAPQIPPNWHIPS |
| | IQDAATPYHPPATPNNMGLIAGAVGGSLLAALVICGIVYWMH |
| | RRTRKAPKRIRLPHIREDDQPSSHQPLFY* |
| gDPolN nucleic | atggggggggctgccgccaggttgggggccgtgattttgttt |
| acid sequence | gtcgtcatagtgggcctccatggggtccgcggcaaatatgcc |
| PolN underlined | ttggcggatgcctctctcaagatggccgaccccaatcgcttt |
| (SEQ ID NO: 17) | cgcggcaaagaccttccggtcctggaccagctgaccgaccct |
| | ccgggggtccggcgcgtgtaccacatccaggcgggcctaccg |
| | gacccgttccagccccccagcctcccgatcacggtttactac |
| | gccgtgttggagcgcgcctgccgcagcgtgctcctaaacgca |
| | ccgtcggaggccccccagattgtccgcggggcctccgaagac |
| | gtccggaaacaaccctacaacctgaccatcgcttggtttcgg |
| | atgggaggcaactgtgctatccccatcacggtcatggagtac |
| | accgaatgctcctacaacaagtctctgggggcctgtcccatc |
| | cgaacgcagccccgctggaactactatgacagcttcagcgcc |
| | gtcagcgaggataacctggggttcctgatgcacgcccccgcg |
| | tttgagaccgccggcacgtacctgcggctcgtgaagataaac |
| | gactggacggagattacacagtttatcctggagcaccgagcc |
| | aagggctcctgtaagtacgccctcccgctgcgcatccccccg |
| | tcagcctgcctctccccccaggcctaccagcagggggtgacg |
| | gtggacagcatcgggatgctgccccgcttcatccccgagaac |
| | cagcgcaccgtcgccgtatacagcttgaagatcgccgggtgg |
| | cacgggccccccctgagctaccagcacttcaggaagctgctg |
| | ctgctggacgaggaggccggccccctggaggaggagctgccc |
| | aggctggccgacgagggcctgaacaggagggtggccgaggac |
| | ctgaacctgggcaacctgaacgtgagcatcccctggacccac |
| | aaggtgggcaacttcaccggcctgtacagcagcaccgtgccc |
| | gtgttcaaccccgagtggcagacccccagcttccccaagatc |
| | cacctgcaggaggacatcgtggacaggtgcaagcagttcgtg |
| | ggtcccctgaccgtgaacgagaagaggaggctgaagctgatc |
| | atgcccgccaggttctaccccaacgtgaccaagtacctgccc |
| | ctggacaagggcatcaagccctactaccccgagcacgccgtg |
| | aaccactacttccagaccaggcactacctgcacaccctgtgg |
| | aaggccggcatcctgtacaagagggagaccaccaggagcgcc |
| | agcttctgcggcagcccctacagctgggagcaggagctgcag |
| | cacggcagctgctggtggctgcagttcaggaacagcaagccc |
| | tgcagcgagtactgcctgacccacctggtgaacctgctggag |
| | gactggggtccctgcgacgagcacggcgagcaccacatcagg |
| | atccccaggacccccgccagggtgaccggcggcgtgttcctg |
| | gtggacaagaacccccacaacaccgccgagagcaggctggtg |
| | gtggacttcagccagttcagcaggggcatcaccagggtgagc |
| | tggcccaagttcgccgtgcccaacctgcagagcctgaccaac |
| | ctgctgagcagcaacctgagctggctgagcctggacgtgagc |
| | gccgccttctaccacatccccctgcaccccgccgccatgccc |
| | gggcccaaggccccatacacgagcaccctgctgcccccggag |
| | ctgtccgagacccccaacgccacgcagccagaactcgccccg |
| | gaagaccccgaggattcggccctcttggaggaccccgtgggg |
| | acggtggcgccgcaaatcccaccaaactggcacatcccgtcg |
| | atccaggacgccgcgacgccttaccatcccccggccaccccg |
| | aacaacatgggcctgatcgccggcgcggtgggcggcagtctc |
| | ctggcagccctggtcatttgcggaattgtgtactggatgcac |
| | cgccgcactcggaaagccccaaagcgcatacgcctcccccac |
| | atccgggaagacgaccagccgtcctcgcaccagcccttgttt |
| | tactag |
| gDPolC amino | MGGAAARLGAVILFVVIVGLHGVRGKYALADASLKMADPNRF |
| acid sequence | RGKDLPVLDQLTDPPGVRRVYHIQAGLPDPFQPPSLPITVYY |
| PolC underlined | AVLERACRSVLLNAPSEAPQIVRGASEDVRKQPYNLTIAWFR |
| (SEQ ID NO: 18) | MGGNCAIPITVMEYTECSYNKSLGACPIRTQPRWNYYDSFSA |
| Signal peptide | VSEDNLGFLMHAPAFETAGTYLRLVKINDWTEITQFILEHRA |
| in italics | KGSCKYALPLRIPPSACLSPQAYQQGVTVDSIGMLPRFIPEN |
| | QRTVAVYSLKIAGWHGPHLLVGSSGLSRYVARLSSNSRIINH |
| | QHGTMQNLHDSCSRNLYVSLLLLYKTFGRKLHLYSHPIILKT |
| | KRWGYSLNFMGYVIGSWGSLPQDHIIQKIKECFRKLPVNRPI |
| | DWKVCQRIVGLLGFAAPFTQCGYPALMPLYACIQSKQAFTFS |
| | PTYKAFLSKQYLNLYPVARQRPGLCQVFADATPTGWGLAMGH |
| | QRMRGTFVAPLPIHTAELLAACFARSRSGAKILGTDNSVVLS |
| | RKYTSFPWLLGCAANWILRGTSFVYVPSALNPADDPSRGRLG |
| | LSRPLLRLPFRPTTGRTSLYAVSPSVGPKAPYTSTLLPPELS |
| | ETPNATQPELAPEDPEDSALLEDPVGTVAPQIPPNWHIPSIQ |
| | DAATPYHPPATPNNMGLIAGAVGGSLLAALVICGIVYWMHRR |
| | TRKAPKRIRLPHIREDDQPSSHQPLFY |
| gDPolC nucleic | atggggggggctgccgccaggttgggggccgtgattttgttt |
| acid sequence | gtcgtcatagtgggcctccatggggtccgcggcaaatatgcc |
| PolC underlined | ttggcggatgcctctctcaagatggccgaccccaatcgcttt |
| (SEQ ID NO: 19) | cgcggcaaagaccttccggtcctggaccagctgaccgaccct |
| | ccgggggtccggcgcgtgtaccacatccaggcgggcctaccg |
| | gacccgttccagccccccagcctcccgatcacggtttactac |
| | gccgtgttggagcgcgcctgccgcagcgtgctcctaaacgca |
| | ccgtcggaggccccccagattgtccgcggggcctccgaagac |
| | gtccggaaacaaccctacaacctgaccatcgcttggtttcgg |
| | atgggaggcaactgtgctatccccatcacggtcatggagtac |
| | accgaatgctcctacaacaagtctctgggggcctgtcccatc |
| | cgaacgcagccccgctggaactactatgacagcttcagcgcc |
| | gtcagcgaggataacctggggttcctgatgcacgcccccgcg |
| | tttgagaccgccggcacgtacctgcggctcgtgaagataaac |
| | gactggacggagattacacagtttatcctggagcaccgagcc |
| | aagggctcctgtaagtacgccctcccgctgcgcatccccccg |
| | tcagcctgcctctccccccaggcctaccagcagggggtgacg |
| | gtggacagcatcgggatgctgccccgcttcatccccgagaac |
| | cagcgcaccgtcgccgtatacagcttgaagatcgccgggtgg |
| | cacgggccccacctgctggtgggcagcagcggcctgagcagg |
| | tacgtggccaggctgagcagcaacagcaggatcatcaaccac |
| | cagcacggcaccatgcagaacctgcacgacagctgcagcagg |
| | aacctgtacgtgagcctgctgctgctgtacaagaccttcggc |
| | aggaagctgcacctgtacagccaccccatcatcctgaagacc |
| | aagaggtggggctacagcctgaacttcatgggctacgtgatc |
| | ggcagctggggcagcctgccccaggaccacatcatccagaag |
| | atcaaggagtgcttcaggaagctgcccgtgaacaggcccatc |
| | gactggaaggtgtgccagaggatcgtgggcctgctgggcttc |
| | gccgcccccttcacccagtgcggctaccccgccctgatgccc |
| | ctgtacgcctgcatccagagcaagcaggccttcaccttcagc |
| | cccacctacaaggccttcctgagcaagcagtacctgaacctg |
| | taccccgtggccaggcagaggcccggcctgtgccaggtgttc |
| | gccgacgccacccccaccggctggggcctggccatgggccac |
| | cagaggatgaggggcaccttcgtggcccccctgcccatccac |
| | accgccgagctgctggccgcctgcttcgccaggagcaggagc |
| | ggcgccaagatcctgggcaccgacaacagcgtggtgetgage |
| | aggaagtacaccagcttcccctggctgctgggctgcgccgcc |
| | aactggatcctgaggggcaccagcttcgtgtacgtgcccagc |
| | gccctgaaccccgccgacgaccccagcaggggcaggctgggc |
| | ctgagcaggcccctgctgaggctgcccttcaggcccaccacc |
| | ggcaggaccagcctgtacgccgtgagccccagcgtggggccc |
| | aaggccccatacacgagcaccctgctgcccccggagctgtcc |
| | gagacccccaacgccacgcagccagaactcgccccggaagac |
| | cccgaggattcggccctcttggaggaccccgtggggacggtg |
| | gcgccgcaaatcccaccaaactggcacatcccgtcgatccag |
| | gacgccgcgacgccttaccatcccccggccaccccgaacaac |
| | atgggcctgatcgccggcgcggtgggcggcagtctcctggca |
| | gccctggtcatttgcggaattgtgtactggatgcaccgccgc |
| | actcggaaagccccaaagcgcatacgcctcccccacatccgg |
| | gaagacgaccagccgtcctcgcaccagcccttgttttactag |
| HBV PolN v2 | HFRKLLLLDEEAGPLEEELPRLADEGLNRRVAEDLNLGNLPE |
| amino acid | WQTPSFPKIHLQEDIVDRCKQFVGPLTVNEKRRLKLIMPARF |
| sequence | YPNVTKYLPLDKGIKPYYPEHAVNHYFQTRHYLHTLWKAGIL |
| (SEQ ID NO: | YKRETTRSASFCGSPYSWEQELQHGSCWWLQFRNSKPCSEYC |
| 173) | LTHLVNLLEDWGPCDEHGEHHIRIPRTPARVT |
| HBV2 amino acid | YLPLDKGIKPYYPEHAVNHYFQTRHYLHTLWKAGILYKRETT |
| sequence | RSASFCGSPYSNEQELQHGSCWNLQFRNSKPCSEYCLTHLVN |
| (SEQ ID NO: | LLEDWGPCDEHGEHHIRIPRTPARVTGGVFLVDKNPHNTAES |
| 174) | RLWDFSQFSRGITRVSWPKFAVPNLQSLTNLLSSNLSVVLSL |
| (Pol N | DVQAFTFSPTYKAFLSKQYLNLYPVARQRPGLCQVFADATPT |
| (italics)-Pol C | GWGLAMGHQRMRGTFVAPLPIHTAELLAACFARSRSGAKILG |
| (underlined)- | TDNSVVLSRKYTSFPWLLGCAANWILRGTSFVYVPSALNPAD |
| Core) | DVGSNLEDPASRELVVSYVNVNMGLKIRQLLWFHISCLTFGR |
| | ETVIEYLVSFGVWIRTPPAYRPPNAPILSTLPETTWRRRDR |
| | GR |
| HBV3 amino acid | HFRKLLLLDEEAGPLEEELPRLADEGLNRRVAEDLNLGNLPE |
| sequence | WQTPSFPKIHLQEDIVDRCKQFVGPLTVNEKRRLKLIMPARF |
| (SEQ ID NO: | YPNVTKYLPLDKGIKPYYPEHAVNHYFQTRHYLHTLWKAGIL |
| 175) | YKRETTRSASFCGSPYSWEQELQHGSCWWLQFRNSKPCSEYC |
| (Pol N | LTHLVNLLEDWGPCDEHGEHHIRIPRTPARVTQAFTFSPTYK |
| (italics)-Pol C | AFLSKQYLNLYPVARQRPGLCQVFADATPTGWGLAMGHQRMR |
| (underlined)- | GTFVAPLPIHTAELLAACFARSRSGAKILGTDNSVVLSRKYT |
| Core) | SFPWLLGCAANWILRGTSFVYVPSALNPADDVGSNLEDPASR |
| | ELWSYVNVNMGLKIRQLLWFHISCLTFGRETVIEYLVSFGV |
| | WIRTPPAYRPPNAPILSTLPETTWRRRDRGR |
| HBV2 nucleic | tatctgccgctggataaaggcattaaaccgtattatccggaa |
| acid sequence | catgcggtgaaccattattttcagacccgccattatctgcat |
| (SEQ ID NO: | accctgtggaaagcgggcattctgtataaacgcgaaaccacc |
| 176) | cgcagcgcgagcttttgcggcagcccgtatagctgggaacag |
| | gaactgcagcatggcagctgctggtggctgcagtttcgcaac |
| | agcaaaccgtgcagcgaatattgcctgacccatctggtgaac |
| | ctgctggaagattggggaccgtgcgatgaacatggcgaacat |
| | catattcgcattccgcgcaccccggcgcgcgtgaccggcggc |
| | gtgtttctggtggataaaaacccgcataacaccgcggaaagc |
| | cgcctggtggtggattttagccagtttagccgcggcattacc |
| | cgcgtgagctggccgaaatttgcggtgccgaacctgcagagc |
| | ctgaccaacctgctgagcagcaacctgagctggctgagcctg |
| | gatgtgcaggcgtttacctUagcccgacctataaagcgtttc |
| | tgagcaaacagtatctgaacctgtatccggtggcgcgccagc |
| | gcccgggcctgtgccaggtgtttgcggatgcgaccccgaccg |
| | gctggggcctggcgatgggccatcagcgcatgcgcggcacct |
| | ttgtggcgccgctgccgattcataccgcggaactgctggcgg |
| | cgtgctttgcgcgcagccgcagcggcgcgaaaattctgggca |
| | ccgataacagcgtggtgctgagccgcaaatataccagctttc |
| | cgtggctgctgggctgcgcggcgaactggattctgcgcggca |
| | ccagctttgtgtatgtgccgagcgcgctgaacccggcggatg |
| | atgtgggcagcaacctggaagatccggcgagccgcgaactgg |
| | tggtgagctatgtgaacgtgaacatgggcctgaaaattcgcc |
| | agctgctgtggtttcatattagctgcctgacctttggccgcg |
| | aaaccgtgattgaatatctggtgagctttggcgtgtggattc |
| | gcaccccgccggcgtatcgcccgccgaacgcgccgattctga |
| | gcaccctgccggaaaccaccgtggtgcgccgccgcgatcggg |
| | gccgc |
| HBV3 nucleic | cattttcgcaaactgctgctgctggatgaagaagcgggaccg |
| acid sequence | ctggaagaagaactgccgcgcctggcggatgaaggcctgaac |
| (SEQ ID NO: | cgccgcgtggcggaagatctgaacctgggcaacctgccggaa |
| 177) | tggcagaccccgagctttccgaaaattcatctgcaggaagat |
| | attgtggatcgctgcaaacagtttgtgggaccgctgaccgtg |
| | aacgaaaaacgccgcctgaaactgattatgccggcgcgcttt |
| | tatccgaacgtgaccaaatatctgccgctggataaaggcatt |
| | aaaccgtattatccggaacatgcggtgaaccattattttcag |
| | acccgccattatctgcataccctgtggaaagcgggcattctg |
| | tataaacgcgaaaccacccgcagcgcgagcttttgcggcagc |
| | ccgtatagctgggaacaggaactgcagcatggcagctgctgg |
| | tggctgcagtttcgcaacagcaaaccgtgcagcgaatattgc |
| | ctgacccatctggtgaacctgctggaagattggggaccgtgc |
| | gatgaacatggcgaacatcatattcgcattccgcgcaccccg |
| | gcgcgcgtgacccaggcgtttacctttagcccgacctataaa |
| | gcgtttctgagcaaacagtatctgaacctgtatccggtggcg |
| | cgccagcgcccgggcctgtgccaggtgtttgcggatgcgacc |
| | ccgaccggctggggcctggcgatgggccatcagcgcatgcgc |
| | ggcacctttgtggcgccgctgccgattcataccgcggaactg |
| | ctggcggcgtgctttgcgcgcagccgcagcggcgcgaaaatt |
| | ctgggcaccgataacagcgtggtgctgagccgcaaatatacc |
| | agctttccgtggctgctgggctgcgcggcgaactggattctg |
| | cgcggcaccagctttgtgtatgtgccgagcgcgctgaacccg |
| | gcggatgatgtgggcagcaacctggaagatccggcgagccgc |
| | gaactggtggtgagctatgtgaacgtgaacatgggcctgaaa |
| | attcgccagctgctgtggtttcatattagctgcctgaccttt |
| | ggccgcgaaaccgtgattgaatatctggtgagctttggcgtg |
| | tggattcgcaccccgccggcgtatcgcccgccgaacgcgccg |
| | attctgagcaccctgccggaaaccaccgtggtgcgccgccga |
| | gatcgaggccgc |
| HBV2 PolN amino | YLPLDKGIKPYYPEHAVNHYFQTRHYLHTLWKAGILYKRETT |
| acid sequence | RSASFCGSPYSWEQELQHGSCWWLQFRNSKPCSEYCLTHLVN |
| (SEQ ID NO: | LLEDWGPCDEHGEHHIRIPRTPARVTGGVFLVDKNPHNTAES |
| 178) | RLVVDFSQFSRGITRVSWPKFAVPNLQSLTNLLSSNLSWLSL |
| | DV |
| HBV2 PolC amino | QAFTFSPTYKAFLSKQYLNLYPVARQRPGLCQVFADATPTGW |
| acid sequence | GLAMGHQRMRGTFVAPLPIHTAELLAACFARSRSGAKILGTD |
| (SEQ ID NO: | NSVVLSRKYTSFPWLLGCAANWILRGTSFVYVPSALNPADD |
| 179) | |
| HBV2 Core amino | VGSNLEDPASRELVVSYVNVNMGLKIRQLLWFHISCLTFGRE |
| acid sequence | TVIEYLVSFGVWIRTPPAYRPPNAPILSTLPETTVVRRRDRG |
| (SEQ ID NO: 180) | R |
| HBV3 PolN amino | HFRKLLLLDEEAGPLEEELPRLADEGLNRRVAEDLNLGNLPE |
| acid sequence | WQTPSFPKIHLQEDIVDRCKQFVGPLTVNEKRRLKLIMPARF |
| (SEQ ID NO: | YPNVTKYLPLDKGIKPYYPEHAVNHYFQTRHYLHTLWKAGIL |
| 181) | YKRETTRSASFCGSPYSWEQELQHGSCWWLQFRNSKPCSEYC |
| | LTHLVNLLEDWGPCDEHGEHHIRIPRTPARVT |
| HBV3 PolC amino | QAFTFSPTYKAFLSKQYLNLYPVARQRPGLCQVFADATPTGW |
| acid sequence | GLAMGHQRMRGTFVAPLPIHTAELLAACFARSRSGAKILGTD |
| (SEQ ID NO: | NSWLSRKYTSFPWLLGCAANWILRGTSFVYVPSALNPADD |
| 182) | |
| HBV3 Core amino | VGSNLEDPASRELWSYVNVNMGLKIRQLLWFHISCLTFGRE |
| acid sequence | TVIEYLVSFGVWIRTPPAYRPPNAPILSTLPETTWRRRDRG |
| (SEQ ID NO: | R |
| 183) | |
| gD-HBV2 nucleic | atggggggggctgccgccaggttgggggccgtgattttgttt |
| acid sequence | gtcgtcatagtgggcctccatggggtccgcggcaaatatgcc |
| (SEQ ID NO: | ttggcggatgcctctctcaagatggccgaccccaatcgcttt |
| 184) | cgcggcaaagaccttccggtcctggaccagctgaccgaccct |
| | ccgggggtccggcgcgtgtaccacatccaggcgggcctaccg |
| | gacccgttccagccccccagcctcccgatcacggtttactac |
| | gccgtgttggagcgcgcctgccgcagcgtgctcctaaacgca |
| | ccgtcggaggccccccagattgtccgcggggcctccgaagac |
| | gtccggaaacaaccctacaacctgaccatcgcttggtttcgg |
| | atgggaggcaactgtgctatccccatcacggtcatggagtac |
| | accgaatgctcctacaacaagtctctgggggcctgtcccatc |
| | cgaacgcagccccgctggaactactatgacagcttcagcgcc |
| | gtcagcgaggataacctggggttcctgatgcacgcccccgcg |
| | tttgagaccgccggcacgtacctgcggctcgtgaagataaac |
| | gactggacggagattacacagtttatcctggagcaccgagcc |
| | aagggctcctgtaagtacgccctcccgctgcgcatccccccg |
| | tcagcctgcctctccccccaggcctaccagcagggggtgacg |
| | gtggacagcatcgggatgctgccccgcttcatccccgagaac |
| | cagcgcaccgtcgccgtatacagcttgaagatcgccgggtgg |
| | cacgggccctatctgccgctggataaaggcattaaaccgtat |
| | tatccggaacatgcggtgaaccattattttcagacccgccat |
| | tatctgcataccctgtggaaagcgggcattctgtataaacgc |
| | gaaaccacccgcagcgcgagcttttgcggcagcccgtatagc |
| | tgggaacaggaactgcagcatggcagctgctggtggctgcag |
| | tttcgcaacagcaaaccgtgcagcgaatattgcctgacccat |
| | ctggtgaacctgctggaagattggggaccgtgcgatgaacat |
| | ggcgaacatcatattcgcattccgcgcaccccggcgcgcgtg |
| | accggcggcgtgtttctggtggataaaaacccgcataacacc |
| | gcggaaagccgcctggtggtggattttagccagtttagccgc |
| | ggcattacccgcgtgagctggccgaaatttgcggtgccgaac |
| | ctgcagagcctgaccaacctgctgagcagcaacctgagctgg |
| | ctgagcctggatgtgcaggcgtttacctttagcccgacctat |
| | aaagcgtttctgagcaaacagtatctgaacctgtatccggtg |
| | gcgcgccagcgcccgggcctgtgccaggtgtttgcggatgcg |
| | accccgaccggctggggcctggcgatgggccatcagcgcatg |
| | cgcggcacctttgtggcgccgctgccgattcataccgcggaa |
| | ctgctggcggcgtgctttgcgcgcagccgcagcggcgcgaaa |
| | attctgggcaccgataacagcgtggtgctgagccgcaaatat |
| | accagctttccgtggctgctgggctgcgcggcgaactggatt |
| | ctgcgcggcaccagctttgtgtatgtgccgagcgcgctgaac |
| | ccggcggatgatgtgggcagcaacctggaagatccggcgagc |
| | cgcgaactggtggtgagctatgtgaacgtgaacatgggcctg |
| | aaaattcgccagctgctgtggtttcatattagctgcctgacc |
| | tttggccgcgaaaccgtgattgaatatctggtgagctttggc |
| | gtgtggattcgcaccccgccggcgtatcgcccgccgaacgcg |
| | ccgattctgagcaccctgccggaaaccaccgtggtgcgccgc |
| | cgcgatcggggccgcgggcccaaggccccatacacgagcacc |
| | ctgctgcccccggagctgtccgagacccccaacgccacgcag |
| | ccagaactcgccccggaagaccccgaggattcggccctcttg |
| | gaggaccccgtggggacggtggcgccgcaaatcccaccaaac |
| | tggcacatcccgtcgatccaggacgccgcgacgccttaccat |
| | cccccggccaccccgaacaacatgggcctgatcgccggcgcg |
| | gtgggcggcagtctcctggcagccctggtcatttgcggaatt |
| | gtgtactggatgcaccgccgcactcggaaagccccaaagcgc |
| | atacgcctcccccacatccgggaagacgaccagccgtcctcg |
| | caccagcccttgttttactag |
| gD-HBV2 amino | MGGAAARLGAVILFVVIVGLHGVRGKYALADASLKMADPNRF |
| acid sequence | RGKDLPVLDQLTDPPGVRRVYHIQAGLPDPFQPPSLPITVYY |
| (SEQ ID NO: | AVLERACRSVLLNAPSEAPQIVRGASEDVRKQPYNLTIAWFR |
| 185) | MGGNCAIPITVMEYTECSYNKSLGACPIRTQPRWNYYDSFSA |
| | VSEDNLGFLMHAPAFETAGTYLRLVKINDWTEITQFILEHRA |
| | KGSCKYALPLRIPPSACLSPQAYQQGVTVDSIGMLPRFIPEN |
| | QRTVAVYSLKIAGWHGPYLPLDKGIKPYYPEHAVNHYFQTRH |
| | YLHTLWKAGILYKRETTRSASFCGSPYSWEQELQHGSCWWLQ |
| | FRNSKPCSEYCLTHLVNLLEDWGPCDEHGEHHIRIPRTPARV |
| | TGGVFLVDKNPHNTAESRLVVDFSQFSRGITRVSWPKFAVPN |
| | LQSLTNLLSSNLSWLSLDVQAFTFSPTYKAFLSKQYLNLYPV |
| | ARQRPGLCQVFADATPTGWGLAMGHQRMRGTFVAPLPIHTAE |
| | LLAACFARSRSGAKILGTDNSVVLSRKYTSFPWLLGCAANWI |
| | LRGTSFVYVPSALNPADDVGSNLEDPASRELVVSYVNVNMGL |
| | KIRQLLWFHISCLTFGRETVIEYLVSFGVWIRTPPAYRPPNA |
| | PILSTLPETTVVRRRDRGRGPKAPYTSTLLPPELSETPNATQ |
| | PELAPEDPEDSALLEDPVGTVAPQIPPNWHIPSIQDAATPYH |
| | PPATPNNMGLIAGAVGGSLLAALVICGIVYWMHRRTRKAPKR |
| | IRLPHIREDDQPSSHQPLFY* |
| gD-HBV3 nucleic | atggggggggctgccgccaggttgggggccgtgattttgttt |
| acid sequence | gtcgtcatagtgggcctccatggggtccgcggcaaatatgcc |
| (SEQ ID NO: | ttggcggatgcctctctcaagatggccgaccccaatcgcttt |
| 186) | cgcggcaaagaccttccggtcctggaccagctgaccgaccct |
| | ccgggggtccggcgcgtgtaccacatccaggcgggcctaccg |
| | gacccgttccagccccccagcctcccgatcacggtttactac |
| | gccgtgttggagcgcgcctgccgcagcgtgctcctaaacgca |
| | ccgtcggaggccccccagattgtccgcggggcctccgaagac |
| | gtccggaaacaaccctacaacctgaccatcgcttggtttcgg |
| | atgggaggcaactgtgctatccccatcacggtcatggagtac |
| | accgaatgctcctacaacaagtctctgggggcctgtcccatc |
| | cgaacgcagccccgctggaactactatgacagcttcagcgcc |
| | gtcagcgaggataacctggggttcctgatgcacgcccccgcg |
| | tttgagaccgccggcacgtacctgcggctcgtgaagataaac |
| | gactggacggagattacacagtttatcctggagcaccgagcc |
| | aagggctcctgtaagtacgccctcccgctgcgcatccccccg |
| | tcagcctgcctctccccccaggcctaccagcagggggtgacg |
| | gtggacagcatcgggatgctgccccgcttcatccccgagaac |
| | cagcgcaccgtcgccgtatacagcttgaagatcgccgggtgg |
| | cacgggccccattttcgcaaactgctgctgctggatgaagaa |
| | gcgggaccgctggaagaagaactgccgcgcctggcggatgaa |
| | ggcctgaaccgccgcgtggcggaagatctgaacctgggcaac |
| | ctgccggaatggcagaccccgagctttccgaaaattcatctg |
| | caggaagatattgtggatcgctgcaaacagtttgtgggaccg |
| | ctgaccgtgaacgaaaaacgccgcctgaaactgattatgccg |
| | gcgcgcttttatccgaacgtgaccaaatatctgccgctggat |
| | aaaggcattaaaccgtattatccggaacatgcggtgaaccat |
| | tattttcagacccgccattatctgcataccctgtggaaagcg |
| | ggcattctgtataaacgcgaaaccacccgcagcgcgagcttt |
| | tgcggcagcccgtatagctgggaacaggaactgcagcatggc |
| | agctgctggtggctgcagtttcgcaacagcaaaccgtgcagc |
| | gaatattgcctgacccatctggtgaacctgctggaagattgg |
| | ggaccgtgcgatgaacatggcgaacatcatattcgcattccg |
| | cgcaccccggcgcgcgtgacccaggcgtttacctttagcccg |
| | acctataaagcgtttctgagcaaacagtatctgaacctgtat |
| | ccggtggcgcgccagcgcccgggcctgtgccaggtgtttgcg |
| | gatgcgaccccgaccggctggggcctggcgatgggccatcag |
| | cgcatgcgcggcacctttgtggcgccgctgccgattcatacc |
| | gcggaactgctggcggcgtgctttgcgcgcagccgcagcggc |
| | gcgaaaattctgggcaccgataacagcgtggtgctgagccgc |
| | aaatataccagctttccgtggctgctgggctgcgcggcgaac |
| | tggattctgcgcggcaccagctttgtgtatgtgccgagcgcg |
| | ctgaacccggcggatgatgtgggcagcaacctggaagatccg |
| | gcgagccgcgaactggtggtgagctatgtgaacgtgaacatg |
| | ggcctgaaaattcgccagctgctgtggtttcatattagctgc |
| | ctgacctttggccgcgaaaccgtgattgaatatctggtgagc |
| | tttggcgtgtggattcgcaccccgccggcgtatcgcccgccg |
| | aacgcgccgattctgagcaccctgccggaaaccaccgtggtg |
| | cgccgccgagatcgaggccgcgggcccaaggccccatacacg |
| | agcaccctgctgcccccggagctgtccgagacccccaacgcc |
| | acgcagccagaactcgccccggaagaccccgaggattcggcc |
| | ctcttggaggaccccgtggggacggtggcgccgcaaatccca |
| | ccaaactggcacatcccgtcgatccaggacgccgcgacgcct |
| | taccatcccccggccaccccgaacaacatgggcctgatcgcc |
| | ggcgcggtgggcggcagtctcctggcagccctggtcatttgc |
| | ggaattgtgtactggatgcaccgccgcactcggaaagcccca |
| | aagcgcatacgcctcccccacatccgggaagacgaccagccg |
| | tcctcgcaccagcccttgttttactag |
| gD-HBV3 amino | MGGAAARLGAVILFVVIVGLHGVRGKYALADASLKMADPNRF |
| acid sequence | RGKDLPVLDQLTDPPGVRRVYHIQAGLPDPFQPPSLPITVYY |
| (SEQ ID NO: | AVLERACRSVLLNAPSEAPQIVRGASEDVRKQPYNLTIAWFR |
| 187) | MGGNCAIPITVMEYTECSYNKSLGACPIRTQPRWNYYDSFSA |
| | VSEDNLGFLMHAPAFETAGTYLRLVKINDWTEITQFILEHRA |
| | KGSCKYALPLRIPPSACLSPQAYQQGVTVDSIGMLPRFIPEN |
| | QRTVAVYSLKIAGWHGPHFRKLLLLDEEAGPLEEELPRLADE |
| | GLNRRVAEDLNLGNLPEWQTPSFPKIHLQEDIVDRCKQFVGP |
| | LTVNEKRRLKLIMPARFYPNVTKYLPLDKGIKPYYPEHAVNH |
| | YFQTRHYLHTLWKAGILYKRETTRSASFCGSPYSWEQELQHG |
| | SCWWLQFRNSKPCSEYCLTHLVNLLEDWGPCDEHGEHHIRIP |
| | RTPARVTQAFTFSPTYKAFLSKQYLNLYPVARQRPGLCQVFA |
| | DATPTGWGLAMGHQRMRGTFVAPLPIHTAELLAACFARSRSG |
| | AKILGTDNSVVLSRKYTSFPWLLGCAANWILRGTSFVYVPSA |
| | LNPADDVGSNLEDPASRELVVSYVNVNMGLKIRQLLWFHISC |
| | LTFGRETVIEYLVSFGVWIRTPPAYRPPNAPILSTLPETTVV |
| | RRRDRGRGPKAPYTSTLLPPELSETPNATQPELAPEDPEDSA |
| | LLEDPVGTVAPQIPPNWHIPSIQDAATPYHPPATPNNMGLIA |
| | GAVGGSLLAALVICGIVYWMHRRTRKAPKRIRLPHIREDDQP |
| | SSHQPLFY* |

### Embodiments

The following list of embodiments is intended to complement, rather than displace or supersede, the previous descriptions.
Embodiment 1. A hepatitis B virus (HBV) Core protein comprising the amino acid sequence of SEQ ID NO: 6 or an immunogenic fragment thereof.
Embodiment 2. The HBV Core protein of embodiment 1, wherein the immunogenic fragment comprises any one of SEQ ID NOs: 20-54.
Embodiment 3. A hepatitis B virus (HBV) Core protein comprising the amino acid sequence of SEQ ID NO: 180 or an immunogenic fragment thereof, or the amino acid sequence of SEQ ID NO: 183 or an immunogenic fragment thereof.
Embodiment 4. A nucleic acid molecule encoding the HBV Core protein of any one of embodiments 1-3.
Embodiment 5. The nucleic acid molecule of embodiment 4, wherein the nucleic acid molecule comprises the nucleotide sequence of SEQ ID NO: 7.
Embodiment 6. A vector comprising the nucleic acid molecule of embodiment 4 or 5.
Embodiment 7. The vector of embodiment 6, wherein the vector is an adenoviral vector.
Embodiment 8. The vector of embodiment 7, wherein the adenoviral vector is an AdC6 vector or AdC7 vector.
Embodiment 9. A vaccine comprising the vector of any one of embodiments 6-8.
Embodiment 10. A HBV polymerase N-terminal domain comprising the amino acid sequence of SEQ ID NO: 8 or an immunogenic fragment thereof.
Embodiment 11. The HBV polymerase N-terminal domain of embodiment 10, wherein the immunogenic fragment comprises any one of SEQ ID NOs: 55-113.
Embodiment 12. A HBV polymerase N-terminal domain comprising the amino acid sequence of SEQ ID NO: 178 or an immunogenic fragment thereof, or the amino acid sequence of SEQ ID NO: 181 or an immunogenic fragment thereof.
Embodiment 13. A HBV polymerase C-terminal domain comprising the amino acid sequence of SEQ ID NO: 10 or an immunogenic fragment thereof.
Embodiment 14. The HBV polymerase C-terminal domain of embodiment 13, wherein the immunogenic fragment comprises any one of SEQ ID NOs: 114-172.
Embodiment 15. A HBV polymerase C-terminal domain comprising the amino acid sequence of SEQ ID NO: 179 or an immunogenic fragment thereof, or the amino acid sequence of SEQ ID NO: 182 or an immunogenic fragment thereof.
Embodiment 16. A nucleic acid molecule encoding the HBV polymerase of any one of embodiments 10-15.
Embodiment 17. The nucleic acid molecule of embodiment 16, wherein the nucleic acid molecule comprises the nucleotide sequence of SEQ ID NO: 9.
Embodiment 18. The nucleic acid molecule of embodiment 16, wherein the nucleic acid molecule comprises the nucleotide sequence of SEQ ID NO: 11.
Embodiment 19. A vector comprising the nucleic acid molecule of any one of embodiments 16-18.
Embodiment 20. The vector of embodiment 19, wherein the vector is an adenoviral vector.
Embodiment 21. The vector of embodiment 20, wherein the adenoviral vector is an AdC6 vector or AdC7 vector.
Embodiment 22. A vaccine comprising the vector of any one of embodiments 19-21.
Embodiment 23. A fusion protein comprising:
   one or more of an HBV Core protein comprising the amino acid sequence of SEQ ID NO: 6 or an immunogenic fragment thereof, an HBV polymerase N-terminal domain comprising the amino acid sequence of SEQ ID NO: 8 or an immunogenic fragment thereof, and an HBV polymerase C-terminal domain comprising the amino acid sequence of SEQ ID NO: 10 or an immunogenic fragment thereof.
Embodiment 24. The fusion protein of embodiment 23, comprising:
   (1) an HBV Core protein comprising the amino acid sequence of SEQ ID NO: 6 or an immunogenic fragment thereof and an HBV polymerase N-terminal domain comprising the amino acid sequence of SEQ ID NO: 8 or an immunogenic fragment thereof;
   (2) one or more of SEQ ID NOs: 20-54 (immunogenic fragments of SEQ ID NO: 6) and one or more of SEQ ID NOs: 55-113 (immunogenic fragments of SEQ ID NO: 8);
   (3) an HBV Core protein comprising the amino acid sequence of SEQ ID NO: 6 or an immunogenic fragment thereof and an HBV polymerase C-terminal domain comprising the amino acid sequence of SEQ ID NO: 10 or an immunogenic fragment thereof;
   (4) one or more of SEQ ID NOs: 20-54 (immunogenic fragments of SEQ ID NO: 6) and one or more of SEQ ID NOs: 114-172 (immunogenic fragments of SEQ ID NO: 10);
   (5) an HBV polymerase N-terminal domain comprising the amino acid sequence of SEQ ID NO: 8 or an immunogenic fragment thereof and an HBV polymerase C-terminal domain comprising the amino acid sequence of SEQ ID NO: 10 or an immunogenic fragment thereof;
   (6) one or more of SEQ ID NOs: 55-113 (immunogenic fragments of SEQ ID NO: 8) and one or more of SEQ ID NOs: 114-172 (immunogenic fragments of SEQ ID NO: 10);
   (7) an HBV Core protein comprising the amino acid sequence of SEQ ID NO: 6 or an immunogenic fragment thereof, an HBV polymerase N-terminal domain comprising the amino acid sequence of SEQ ID NO: 8 or an immunogenic fragment thereof, and an HBV polymerase C-terminal domain comprising the amino acid sequence of SEQ ID NO: 10 or an immunogenic fragment thereof; or
   (8) one or more of SEQ ID NOs: 20-54 (immunogenic fragments of SEQ ID NO: 6), one or more of SEQ ID NOs: 55-113 (immunogenic fragments of SEQ ID NO: 8), and one or more of SEQ ID NOs: 114-172 (immunogenic fragments of SEQ ID NO: 10).
Embodiment 25. A fusion protein comprising:
   an HBV polymerase N-terminal domain comprising the amino acid sequence of SEQ ID NO: 178 or an immunogenic fragment thereof, an HBV polymerase C-terminal domain comprising the amino acid sequence of SEQ ID NO: 179 or an immunogenic fragment thereof, and an HBV Core protein comprising the amino acid sequence of SEQ ID NO: 180 or an immunogenic fragment thereof.
Embodiment 26. The fusion protein of embodiment 25, comprising the amino acid sequence of SEQ ID NO: 174.
Embodiment 27. A fusion protein comprising:
   an HBV polymerase N-terminal domain comprising the amino acid sequence of SEQ ID NO: 181 or an immunogenic fragment thereof, an HBV polymerase C-terminal domain comprising the amino acid sequence of SEQ ID NO: 182 or an immunogenic fragment thereof, and an HBV Core protein comprising the amino acid sequence of SEQ ID NO: 183 or an immunogenic fragment thereof.
Embodiment 28. The fusion protein of embodiment 27, comprising the amino acid sequence of SEQ ID NO: 175.
Embodiment 29. A fusion protein comprising:
   an N-terminal herpes simplex virus (HSV) glycoprotein (gD) sequence or a variant thereof;
   an HBV Core protein comprising the amino acid sequence of SEQ ID NO: 6 or an immunogenic fragment thereof; and
   a C-terminal HSV gD sequence or a variant thereof.
Embodiment 30. The fusion protein of embodiment 29, wherein the immunogenic fragment comprises any one of SEQ ID NOs: 20-54.
Embodiment 31. A fusion protein comprising:
   an N-terminal HSV gD sequence or a variant thereof;
   an HBV polymerase N-terminal domain comprising the amino acid sequence of SEQ ID NO: 8 or an immunogenic fragment thereof; and
   a C-terminal HSV gD protein sequence or a variant thereof.
Embodiment 32. The fusion protein of embodiment 31, wherein the immunogenic fragment comprises any one of SEQ ID NOs: 55-113.
Embodiment 33. A fusion protein comprising:
   an N-terminal HSV gD sequence or a variant thereof;
   an HBV polymerase C-terminal domain comprising the amino acid sequence of SEQ ID NO: 10 or an immunogenic fragment thereof; and
   a C-terminal HSV gD protein sequence or a variant thereof.
Embodiment 34. The fusion protein of embodiment 33, wherein the immunogenic fragment comprises any one of SEQ ID NOs: 114-172.
Embodiment 35. A fusion protein comprising:
   an N-terminal HSV gD sequence or a variant thereof;
   an HBV sequence comprising:
(1) an HBV Core protein comprising the amino acid sequence of SEQ ID NO: 6 or an immunogenic fragment thereof and an HBV polymerase N-terminal domain comprising the amino acid sequence of SEQ ID NO: 8 or an immunogenic fragment thereof;
(2) one or more of SEQ ID NOs: 20-54 (immunogenic fragments of SEQ ID NO: 6) and one or more of SEQ ID NOs: 55-113 (immunogenic fragments of SEQ ID NO: 8);
(3) an HBV Core protein comprising the amino acid sequence of SEQ ID NO: 6 or an immunogenic fragment thereof and an HBV polymerase C-terminal domain comprising the amino acid sequence of SEQ ID NO: 10 or an immunogenic fragment thereof;
(4) one or more of SEQ ID NOs: 20-54 (immunogenic fragments of SEQ ID NO: 6) and one or more of SEQ ID NOs: 114-172 (immunogenic fragments of SEQ ID NO: 10);
(5) an HBV polymerase N-terminal domain comprising the amino acid sequence of SEQ ID NO: 8 or an immunogenic fragment thereof and an HBV polymerase C-terminal domain comprising the amino acid sequence of SEQ ID NO: 10 or an immunogenic fragment thereof;
(6) one or more of SEQ ID NOs: 55-113 (immunogenic fragments of SEQ ID NO: 8) and one or more of SEQ ID NOs: 114-172 (immunogenic fragments of SEQ ID NO: 10);
(7) an HBV Core protein comprising the amino acid sequence of SEQ ID NO: 6 or an immunogenic fragment thereof, an HBV polymerase N-terminal domain comprising the amino acid sequence of SEQ ID NO: 8 or an immunogenic fragment thereof, and an HBV polymerase C-terminal domain comprising the amino acid sequence of SEQ ID NO: 10 or an immunogenic fragment thereof; or
(8) one or more of SEQ ID NOs: 20-54 (immunogenic fragments of SEQ ID NO: 6), one or more of SEQ ID NOs: 55-113 (immunogenic fragments of SEQ ID NO: 8), and one or more of SEQ ID NOs: 114-172 (immunogenic fragments of SEQ ID NO: 10) and
a C-terminal HSV gD protein sequence or a variant thereof.
Embodiment 36. A fusion protein comprising:
   an N-terminal HSV gD sequence or a variant thereof;
   an HBV Core protein comprising the amino acid sequence of SEQ ID NO: 180 or an immunogenic fragment thereof, or the amino acid sequence of SEQ ID NO: 183 or an immunogenic fragment thereof; and
   a C-terminal HSV gD sequence or a variant thereof.
Embodiment 37. A fusion protein comprising:
   an N-terminal HSV gD sequence or a variant thereof;
   an HBV polymerase N-terminal domain comprising the amino acid sequence of SEQ ID NO: 178 or an immunogenic fragment thereof, or the amino acid sequence of SEQ ID NO: 181 or an immunogenic fragment thereof; and
   a C-terminal HSV gD protein sequence or a variant thereof.
Embodiment 38. A fusion protein comprising:
   an N-terminal HSV gD sequence or a variant thereof;
   an HBV polymerase C-terminal domain comprising the amino acid sequence of SEQ ID NO: 179 or an immunogenic fragment thereof, or the amino acid sequence of SEQ ID NO: 182 or an immunogenic fragment thereof; and
   a C-terminal HSV gD protein sequence or a variant thereof.
Embodiment 39. A fusion protein comprising:
   an N-terminal HSV gD sequence or a variant thereof;
   an HBV sequence comprising:
(1) an HBV polymerase N-terminal domain comprising the amino acid sequence of SEQ ID NO: 178 or an immunogenic fragment thereof, an HBV polymerase C-terminal domain comprising the amino acid sequence of SEQ ID NO: 179 or an immunogenic fragment thereof, and an HBV Core protein comprising the amino acid sequence of SEQ ID NO: 180 or an immunogenic fragment thereof; or
(2) an HBV polymerase N-terminal domain comprising the amino acid sequence of SEQ ID NO: 181 or an immunogenic fragment thereof, an HBV polymerase C-terminal domain comprising the amino acid sequence of SEQ ID NO: 182 or an immunogenic fragment thereof, and an HBV Core protein comprising the amino acid sequence of SEQ ID NO: 183 or an immunogenic fragment thereof; and
a C-terminal HSV gD protein sequence or a variant thereof.
Embodiment 40. The fusion protein of embodiment 39, wherein the HBV sequence comprises an HBV polymerase N-terminal domain comprising the amino acid sequence of SEQ ID NO: 178 or an immunogenic fragment thereof, an HBV polymerase C-terminal domain comprising the amino acid sequence of SEQ ID NO: 179 or an immunogenic fragment thereof, and an HBV Core protein comprising the amino acid sequence of SEQ ID NO: 180 or an immunogenic fragment thereof.
Embodiment 41. The fusion protein of embodiment 40, wherein the HBV sequence comprises the amino acid sequence of SEQ ID NO: 174.
Embodiment 42. The fusion protein of embodiment 39, wherein the HBV sequence comprises an HBV polymerase N-terminal domain comprising the amino acid sequence of SEQ ID NO: 181 or an immunogenic fragment thereof, an HBV polymerase C-terminal domain comprising the amino acid sequence of SEQ ID NO: 182 or an immunogenic fragment thereof, and an HBV Core protein comprising the amino acid sequence of SEQ ID NO: 183 or an immunogenic fragment thereof
Embodiment 43. The fusion protein of embodiment 42, wherein the HBV sequence comprises the amino acid sequence of SEQ ID NO: 175.
Embodiment 44. The fusion protein of any one of embodiments 29-43, wherein the N-terminal HSV gD sequence comprises the amino acid sequence of SEQ ID NO: 12.
Embodiment 45. The fusion protein of any one of embodiments 29-43, wherein the N-terminal HSV gD sequence comprises amino acid residues 26-269 of SEQ ID NO: 12.
Embodiment 46. The fusion protein of any one of embodiments 29-45, wherein the C-terminal HSV gD sequence comprises the transmembrane domain of the HSV gD.
Embodiment 47. The fusion protein of any one of embodiments 29-46, wherein the C-terminal HSV gD sequence comprises the amino acid sequence of SEQ ID NO: 13.
Embodiment 48. The fusion protein of any one of embodiments 29-47, wherein the fusion protein comprises the amino acid sequence of any one of SEQ ID NO: 14 or an immunogenic fragment thereof, SEQ ID NO: 16 or an immunogenic fragment thereof, or SEQ ID NO: 18 or an immunogenic fragment thereof.
Embodiment 49. The fusion protein of any one of embodiments 39-47, wherein the fusion protein comprises the amino acid sequence of SEQ ID NO: 185.
Embodiment 50. The fusion protein of any one of embodiments 39-47, wherein the fusion protein comprises the amino acid sequence of SEQ ID NO: 187.
Embodiment 51. A nucleic acid molecule encoding the fusion protein of any one of embodiments 23-50.
Embodiment 52. The nucleic acid molecule of embodiment 51, wherein the nucleic acid molecule comprises the nucleotide sequence of any one of SEQ ID NOs: 15, 17, or 19.
Embodiment 53. The nucleic acid molecule of embodiment 51, wherein the nucleic acid molecule comprises the nucleotide sequence of SEQ ID NO: 176.
Embodiment 54. The nucleic acid molecule of embodiment 51, wherein the nucleic acid molecule comprises the nucleotide sequence of SEQ ID NO: 177.
Embodiment 55. The nucleic acid molecule of embodiment 51, wherein the nucleic acid molecule comprises the nucleotide sequence of SEQ ID NO: 184.
Embodiment 56. The nucleic acid molecule of embodiment 51, wherein the nucleic acid molecule comprises the nucleotide sequence of SEQ ID NO: 186.
Embodiment 57. A vector comprising the nucleic acid molecule of any one of embodiments 51-56.
Embodiment 58. The vector of embodiment 57, wherein the vector is an adenoviral vector.
Embodiment 59. The vector of embodiment 58, wherein the adenoviral vector is an AdC6 vector or AdC7 vector.
Embodiment 60. A vaccine comprising the vector of any one of embodiments 57-59.
Embodiment 61. A method of inducing an immune response to HBV in a subject, the method comprising providing to the subject an effective amount of the fusion protein of any one of embodiments 23-50, the nucleic acid molecule of any one of embodiments 51-56, the vector of any one of embodiments 57-59, or the vaccine of embodiment 60 to thereby induce an immune response to HBV.
Embodiment 62. The method of embodiment 61, wherein the vaccine comprises an AdC6 vector comprising a fusion protein comprising the amino acid sequence of any one of SEQ ID NOs: 14, 16, or 18, or an immunogenic fragment thereof.
Embodiment 63. The method of embodiment 62, further comprising providing to the subject, subsequent to providing the vaccine comprising the AdC6 vector, a vaccine comprising an AdC7 vector comprising a fusion protein comprising the amino acid sequence of any one of SEQ ID NOs: 14, 16, or 18, or an immunogenic fragment thereof.
Embodiment 64. The method of embodiment 61, wherein the vaccine comprises an AdC7 vector comprising a fusion protein comprising the amino acid sequence of any one of SEQ ID NOs: 14, 16, or 18, or an immunogenic fragment thereof.
Embodiment 65. The method of embodiment 64, further comprising providing to the subject, subsequent to providing the vaccine comprising the AdC7 vector, a vaccine comprising an AdC6 vector comprising a fusion protein comprising the amino acid sequence of any one of SEQ ID NOs: 14, 16, or 18, or an immunogenic fragment thereof.
Embodiment 66. The method of embodiment 61, wherein the vaccine comprises an AdC6 vector comprising a fusion protein comprising the amino acid sequence of SEQ ID NO: 185.
Embodiment 67. The method of embodiment 66, further comprising providing to the subject, subsequent to providing the vaccine comprising the AdC6 vector, a vaccine comprising an AdC7 vector comprising a fusion protein comprising the amino acid sequence of SEQ ID NO: 185.
Embodiment 68. The method of embodiment 61, wherein the vaccine comprises an AdC7 vector comprising a fusion protein comprising the amino acid sequence of SEQ ID NO: 185.
Embodiment 69. The method of embodiment 68, further comprising providing to the subject, subsequent to providing the vaccine comprising the AdC7 vector, a vaccine comprising an AdC6 vector comprising a fusion protein comprising the amino acid sequence of SEQ ID NO: 185.
Embodiment 70. The method of embodiment 61, wherein the vaccine comprises an AdC6 vector comprising a fusion protein comprising the amino acid sequence of SEQ ID NO: 187.
Embodiment 71. The method of embodiment 70, further comprising providing to the subject, subsequent to providing the vaccine comprising the AdC6 vector, a vaccine comprising an AdC7 vector comprising a fusion protein comprising the amino acid sequence of SEQ ID NO: 187.
Embodiment 72. The method of embodiment 61, wherein the vaccine comprises an AdC7 vector comprising a fusion protein comprising the amino acid sequence of SEQ ID NO: 187.
Embodiment 73. The method of embodiment 72, further comprising providing to the subject, subsequent to providing the vaccine comprising the AdC7 vector, a vaccine comprising an AdC6 vector comprising a fusion protein comprising the amino acid sequence of SEQ ID NO: 187.
Embodiment 74. The method of any one of embodiments 61-73, wherein the amino acid sequence of any one of SEQ ID NOs: 14, 16, 18, 185, or 187, or an immunogenic fragment thereof, does not contain the N-terminal 25 amino acid signal peptide.

What is claimed:

1. A method of inducing an immune response to HBV in a subject, the method comprising:
providing to the subject an effective amount of a first vaccine comprising a nucleic acid molecule that comprises a nucleotide sequence encoding an HBV polymerase N-terminal domain comprising the amino acid sequence of SEQ ID NO: 178 or an immunogenic fragment thereof, a nucleotide sequence encoding an HBV polymerase C-terminal domain comprising the amino acid sequence of SEQ ID NO: 179 or an immunogenic fragment thereof, and a nucleotide sequence encoding an HBV Core protein comprising the amino acid sequence of SEQ ID NO: 180 or an immunogenic fragment thereof, and
providing to the subject an effective amount of a second vaccine comprising a nucleic acid molecule that comprises a nucleotide sequence encoding an HBV polymerase N-terminal domain comprising the amino acid sequence of SEQ ID NO: 178 or an immunogenic fragment thereof, a nucleotide sequence encoding an HBV polymerase C-terminal domain comprising the amino acid sequence of SEQ ID NO: 179 or an immunogenic fragment thereof, and a nucleotide sequence encoding an HBV Core protein comprising the amino acid sequence of SEQ ID NO: 180 or an immunogenic fragment thereof,
to thereby induce an immune response to HBV.

2. The method of claim 1, wherein the nucleic acid molecule of the first vaccine encodes a fusion protein comprising the amino acid sequence of SEQ ID NO: 174, the nucleic acid molecule of the second vaccine encodes a fusion protein comprising the amino acid sequence of SEQ ID NO:
174, or the nucleic acid molecule of the first vaccine and the nucleic acid molecule of the second vaccine encode a fusion protein comprising the amino acid sequence of SEQ ID NO: 174.

3. The method of claim 1, wherein the nucleic acid molecule of the first vaccine, the nucleic acid molecule of the second vaccine, or the nucleic acid molecule of the first vaccine and the nucleic acid molecule of the second vaccine further comprise a nucleotide sequence encoding:
an N-terminal HSV gD sequence or a variant thereof,
a C-terminal HSV gD sequence or a variant thereof, or
both.

4. The method of claim 3, comprising:
providing to the subject an effective amount of a first vaccine comprising a nucleic acid molecule that comprises a nucleotide sequence encoding an N-terminal HSV gD sequence or a variant thereof, a nucleotide sequence encoding an HBV polymerase N-terminal domain comprising the amino acid sequence of SEQ ID NO: 178 or an immunogenic fragment thereof, a nucleotide sequence encoding an HBV polymerase C-terminal domain comprising the amino acid sequence of SEQ ID NO: 179 or an immunogenic fragment thereof, a nucleotide sequence encoding an HBV Core protein comprising the amino acid sequence of SEQ ID NO: 180 or an immunogenic fragment thereof, and a nucleotide sequence encoding a C-terminal HSV gD sequence or a variant thereof, and
providing to the subject an effective amount of a second vaccine comprising a nucleic acid molecule that comprises a nucleotide sequence encoding an N-terminal HSV gD sequence or a variant thereof, a nucleotide sequence encoding an HBV polymerase N-terminal domain comprising the amino acid sequence of SEQ ID NO: 178 or an immunogenic fragment thereof, a nucleotide sequence encoding an HBV polymerase C-terminal domain comprising the amino acid sequence of SEQ ID NO: 179 or an immunogenic fragment thereof, a nucleotide sequence encoding an HBV Core protein comprising the amino acid sequence of SEQ ID NO: 180 or an immunogenic fragment thereof, and a nucleotide sequence encoding a C-terminal HSV gD sequence or a variant thereof,
to thereby induce an immune response to HBV.

5. The method of claim 4, wherein the nucleic acid molecule of the first vaccine, the nucleic acid molecule of the second vaccine, or the nucleic acid molecule of the first vaccine and the nucleic acid molecule of the second vaccine encode an HBV fusion protein comprising the amino acid sequence of SEQ ID NO: 174.

6. The method of claim 5, wherein the N-terminal HSV gD sequence of the first vaccine, the N-terminal HSV gD sequence of the second vaccine, or the N-terminal HSV gD sequence of the first vaccine and the N-terminal HSV gD sequence of the second vaccine comprise the amino acid sequence of SEQ ID NO: 12.

7. The method of claim 5, wherein the N-terminal HSV gD sequence of the first vaccine, the N-terminal HSV gD sequence of the second vaccine, or the N-terminal HSV gD sequence of the first vaccine and the N-terminal HSV gD sequence of the second vaccine comprise amino acid residues 26-269 of SEQ ID NO: 12.

8. The method of claim 5, wherein the C-terminal HSV gD sequence of the first vaccine, the C-terminal HSV gD sequence of the second vaccine, or the C-terminal HSV gD sequence of the first vaccine and the C-terminal HSV gD sequence of the second vaccine comprise the transmembrane domain of the HSV gD.

9. The method of claim 8, wherein the C-terminal HSV gD sequence of the first vaccine, the C-terminal HSV gD sequence of the second vaccine, or the C-terminal HSV gD sequence of the first vaccine and the C-terminal HSV gD sequence of the second vaccine comprise the amino acid sequence of SEQ ID NO: 13.

10. The method of claim 4, comprising:
providing to the subject an effective amount of a first vaccine comprising a nucleic acid molecule that comprises a nucleotide sequence encoding an N-terminal HSV gD sequence comprising amino acid residues 26-269 of SEQ ID NO: 12, a nucleotide sequence encoding an HBV polymerase N-terminal domain comprising the amino acid sequence of SEQ ID NO: 178 or an immunogenic fragment thereof, a nucleotide sequence encoding an HBV polymerase C-terminal domain comprising the amino acid sequence of SEQ ID NO: 179 or an immunogenic fragment thereof, a nucleotide sequence encoding an HBV Core protein comprising the amino acid sequence of SEQ ID NO: 180 or an immunogenic fragment thereof, and a nucleotide sequence encoding a C-terminal HSV gD sequence comprising the amino acid sequence of SEQ ID NO: 13, and
providing to the subject an effective amount of a second vaccine comprising a nucleic acid molecule that comprises a nucleotide sequence encoding an N-terminal HSV gD sequence comprising amino acid residues 26-269 of SEQ ID NO: 12, a nucleotide sequence encoding an HBV polymerase N-terminal domain comprising the amino acid sequence of SEQ ID NO: 178 or an immunogenic fragment thereof, a nucleotide sequence encoding an HBV polymerase C-terminal domain comprising the amino acid sequence of SEQ ID NO: 179 or an immunogenic fragment thereof, a nucleotide sequence encoding an HBV Core protein comprising the amino acid sequence of SEQ ID NO: 180 or an immunogenic fragment thereof, and a nucleotide sequence encoding a C-terminal HSV gD sequence comprising the amino acid sequence of SEQ ID NO: 13,
to thereby induce an immune response to HBV.

11. The method of claim 10, wherein the N-terminal HSV gD sequence of the first vaccine, the N-terminal HSV gD sequence of the second vaccine, or the N-terminal HSV gD sequence of the first vaccine and the N-terminal HSV gD sequence of the second vaccine comprise the amino acid sequence of SEQ ID NO: 12.

12. The method of claim 10, wherein the nucleic acid molecule of the first vaccine, the nucleic acid molecule of the second vaccine, or the nucleic acid molecule of the first vaccine and the nucleic acid molecule of the second vaccine encode an HBV fusion protein comprising the amino acid sequence of SEQ ID NO: 174.

13. The method of claim 12, wherein the nucleic acid molecule of the first vaccine, the nucleic acid molecule of the second vaccine, or the nucleic acid molecule of the first vaccine and the nucleic acid molecule of the second vaccine comprise the nucleotide sequence of SEQ ID NO: 176.

14. The method of claim 10, wherein the nucleic acid molecule of the first vaccine, the nucleic acid molecule of the second vaccine, or the nucleic acid molecule of the first vaccine and the nucleic acid molecule of the second vaccine encode a fusion protein comprising the amino acid sequence of SEQ ID NO: 185.

15. The method of claim 14, wherein the nucleic acid molecule of the first vaccine, the nucleic acid molecule of the second vaccine, or the nucleic acid molecule of the first vaccine and the nucleic acid molecule of the second vaccine comprise the nucleotide sequence of SEQ ID NO: 184.

16. The method of claim 14, wherein the first vaccine comprises an AdC6 vector comprising a nucleic acid molecule encoding a fusion protein comprising the amino acid sequence of SEQ ID NO: 185.

17. The method of claim 16, comprising
providing to the subject an effective amount of a first vaccine comprising an AdC6 vector comprising a nucleic acid molecule encoding a fusion protein comprising the amino acid sequence of SEQ ID NO: 185, and
subsequent to providing the first vaccine, providing to the subject an effective amount of a second vaccine comprising an AdC7 vector comprising a nucleic acid molecule encoding a fusion protein comprising the amino acid sequence of SEQ ID NO: 185,
to thereby induce an immune response to HBV.

18. The method of claim 14, wherein the first vaccine comprises an AdC7 vector comprising a nucleic acid molecule encoding a fusion protein comprising the amino acid sequence of SEQ ID NO: 185.

19. The method of claim 18, comprising
providing to the subject an effective amount of a first vaccine comprising an AdC7 vector comprising a nucleic acid molecule encoding a fusion protein comprising the amino acid sequence of SEQ ID NO: 185, and
subsequent to providing the first vaccine, providing to the subject an effective amount of a second vaccine comprising an AdC6 vector comprising a nucleic acid molecule encoding a fusion protein comprising the amino acid sequence of SEQ ID NO: 185,
to thereby induce an immune response to HBV.

20. The method of claim 14, wherein the nucleic acid molecule of the first vaccine, the nucleic acid molecule of the second vaccine, or the nucleic acid molecule of the first vaccine and the nucleic acid molecule of the second vaccine encode a fusion protein comprising the amino acid sequence of SEQ ID NO: 185, or an immunogenic fragment thereof, without the N-terminal 25 amino acid signal peptide.
